(19) United States
(12) Patent Application Publication
(10) Pub. No.: US 2025/0051418 A1
(43) Pub. Date: Feb. 13, 2025

(54) VIRAL VECTORS AND USE THEREOF IN ADOPTIVE CELLULAR THERAPY

(71) Applicant: Immatics US, Inc., Houston, TX (US)

(72) Inventors: Melinda MATA, Houston, TX (US); Yannick BULLIARD, Houston, TX (US); Mamta KALRA, Houston, TX (US)

(21) Appl. No.: 18/590,008

(22) Filed: Feb. 28, 2024

Related U.S. Application Data
(63) Continuation of application No. 16/884,804, filed on May 27, 2020, now Pat. No. 11,975,025.
(60) Provisional application No. 62/853,123, filed on May 27, 2019.

(51) Int. Cl. C07K 14/705; A61K 39/00; C07K 14/725; C12N 5/078; C12N 5/0783; C12N 15/64; C12N 15/86

(52) U.S. Cl. CPC C07K 14/70517; A61K 39/4611; A61K 39/4632; A61K 39/46449; C07K 14/7051; C12N 5/0634; C12N 5/0636; C12N 15/64; C12N 15/86; C12N 2501/2302; C12N 2501/2315; C12N 2740/16043

(57) ABSTRACT

A vector containing a first nucleotide sequence S1 encoding a protein Z1, a second nucleotide sequence S2 encoding a protein Z2, a third nucleotide sequence S3 encoding a protein Y1, and a fourth nucleotide sequence S4 encoding a protein Y2, in which Z1 and Z2 form a first dimer and Y1 and Y2 form a second dimer, in which the first dimer Z1Z2 is different from the second dimer Y1Y2.

### CROSS-REFERENCE TO RELATED APPLICATION

This application is a Continuation application of U.S. application Ser. No. 16/884,804, filed May 27, 2020, which claims the benefit of U.S. Provisional Application No. 62/853,123, filed May 27, 2019, the contents of each which are incorporated herein by reference in their entirety.

### REFERENCE TO SEQUENCE LISTING SUBMITTED AS AN XML FILE (.XML)

A Sequence Listing is submitted herewith as an XML file named 3000011-013002_Sequence_Listing.xml created on 12 Feb. 2024 and having a size of 323,355 bytes as permitted under 37 C.F.R. § 1.821 (c). The material in the aforementioned file is hereby incorporated by reference in its entirety.

### BACKGROUND

### 1. Field

The present disclosure relates to T cell manufacturing. In an aspect, the present disclosure relates to T cell manufacturing using a multi-cistronic cassette for expressing a plurality of proteins in a single vector. More specifically, the present disclosure relates to T cell manufacturing of T cells that co-express TCRαβ and CD8αβ and the use thereof in adoptive cellular therapy.

### 2. Background

The genetic engineering of human lymphocytes as a potential therapy for inherited, acquired or infectious disease requires efficient transfer and expression of the transgenes. In the case of adoptive immunotherapy for cancer, naturally-occurring and/or recombinant antitumor T-cell receptors (TCRs) have been used to endow normal T cells or tumor infiltrating lymphocytes with antitumor reactivity.

Morgan et al. (J Immunol. 2003 Sep. 15; 171 (6): 3287-32 percent) discloses an anti-gp 100 TCR expressed by a bicistronic RNA, in which the expression of the first gene encoding the TCRβ chain is controlled by a long terminal repeat (LTR) and the second gene encoding the TCRα chain is governed by an internal ribosome entry site (IRES). CD4+ T cells engineered with this anti-gp100 TCR gene were antigen reactive.

Cohen et al. (J Immunol. 2005 Nov. 1; 175 (9): 5799-5808) discloses a bicistronic retroviral vector for co-expression of both TCRα chain and TCRβ chain that bind a p53 epitope. The expression of the first gene encoding the TCRα chain is controlled by an LTR and the second gene encoding the TCRβ chain is governed by an IRES. The p53 TCR-transduced lymphocytes were able to specifically recognize, with high-avidity, peptide-pulsed APCs as well as HLA-A2.1+ cells transfected with either wild-type or mutant p53 protein.

Hughes et al. (Hum Gene Ther. 2005 April; 16 (4): 457-472) discloses various bicistronic retroviral vectors for co-expression of an anti-MART-1 TCR. The expression of the first gene encoding the TCRα chain is controlled by an LTR and the second gene encoding the TCRβ chain is governed by an IRES, or vice versa. In addition, the expression of the first gene encoding the TCRα chain is controlled by an LTR and the second gene encoding the TCRβ chain is governed by a PGK promoter, or vice versa. T cells transduced with these vectors showed highly active T cell effector functions.

Zhao et al. (J Immunol. 2005 Apr. 1; 174 (7): 4415-4423) discloses bicistronic retroviral vectors for co-expression of NY-ESO-1 TCR. The expression of the first gene encoding the TCRα chain is controlled by an LTR and the second gene encoding the TCRβ chain is governed by an IRES, or the expression of the first gene encoding the TCRα chain is controlled by an LTR and the second gene encoding the TCRβ chain is governed by a PGK promoter. The transduced lymphocytes could efficiently recognize and kill HLA-A2- and NY-ESO-1-positive melanoma cell lines.

Morgan et al. (Gene Therapy (2008) 15, 1411-1423) discloses bicistronic lentiviral vectors that combine a furin cleavage site and an amino acid spacer (GSG or SGSG (SEQ ID NO: 8)) followed by a 2A ribosomal skip peptide to express an anti-gp100 TCR or an anti-MART-1 TCR. When the spacer sequence was augmented by the addition of a synthetic V5 peptide tag sequence protein processing was boosted, which resulted in a lentiviral vector capable of mediating high-level TCR expression in transduced lymphocytes.

There remains a need for gene delivery systems for safe and efficient transgene expression in adoptive cellular therapy.

### BRIEF SUMMARY

In an aspect, the disclosure provide for a gene delivery system including a vector comprising a first nucleotide sequence S1 encoding a protein Z1, a second nucleotide sequence S2 encoding a protein Z2, a third nucleotide sequence S3 encoding a protein Y1, and a fourth nucleotide sequence S4 encoding a protein Y2, in which Z1 and Z2 form a first dimer and Y1 and Y2 form a second dimer, in which the first dimer Z1Z2 is different from the second dimer Y1Y2 and wherein the gene delivery system is used in adaptive cellular therapy.

In another aspect, the S1, S2, S3, and S4 may be arranged in tandem in a 5′ to 3′ orientation selected from S1-S2-S3-S4, S1-S2-S4-S3, S1-S3-S2-S4, S1-S3-S4-S2, S1-S4-S3-S2, S1-S4-S2-S3, S2-S1-S3-S4, S2-S1-S4-S3, S2-S3-S1-S4, S2-S3-S4-S1, S2-S4-S3-S1, S2-S4-S1-S3, S3-S1-S2-S4, S3-S1-S4-S2, S3-S2-S1-S4, S3-S2-S4-S1, S3-S4-S1-S2, S3-S4-S2-S1, S4-S1-S2-S3, S4-S1-S3-S2, S4-S2-S1-S3, S4-S2-S3-S1, S4-S3-S1-S2, or S4-S3-S2-S1.

In another aspect, the vector may further include a fifth nucleotide sequence S5 encoding a 2A peptide and a sixth nucleotide sequence S6 encoding a linker peptide, wherein S5 and S6 are positioned between S1 and S2, S1 and S3, S1 and S4, S2 and S3, S2 and S4, and/or S3 and S4.

In another aspect, the 2A peptide may be selected from P2A (SEQ ID NO: 3), T2A (SEQ ID NO: 4), E2A (SEQ ID NO: 5), or F2A (SEQ ID NO: 6).

In another aspect, the linker peptide may be GSG or SGSG (SEQ ID NO: 8).

In another aspect, the vector may include a seventh nucleotide sequence S7 encoding a furin peptide (SEQ ID NO: 2), wherein S7 is positioned between S1 and S2, S1 and S3, S1 and S4, S2 and S3, S2 and S4, and/or S3 and S4.

In another aspect, the vector may further include a post-transcriptional regulatory element (PRE) sequence selected from a Woodchuck PRE (WPRE) or a hepatitis B virus (HBV) PRE (HPRE).

In another aspect, the vector may further include a promoter sequence that controls the transcription of S1, S2, S3, S4, S5, S6 and/or S7, wherein the promoter sequence is selected from cytomegalovirus (CMV) promoter, phosphoglycerate kinase (PGK) promoter, myelin basic protein (MBP) promoter, glial fibrillary acidic protein (GFAP) promoter, modified MoMuLV LTR containing myeloproliferative sarcoma virus enhancer (MNDU3), Ubiqitin C promoter, EF-1 alpha promoter, or Murine Stem Cell Virus (MSCV) promoter.

In another aspect, the first dimer Z1Z2 may be selected from SEQ ID NO: 13 and 14, 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 72, 73 and 74, 75 and 76, 77 and 78, 79 and 80, 81 and 82, 83 and 84, 85 and 86, 87 and 88, or 89 and 90.

In another aspect, the second dimer Y1 and Y2 is set forth in SEQ ID NO: 11 and 12.

In another aspect, the orientation is S2-S1-S4-S3.

In another aspect, the vector has the sequence selected from PTE WPRE (SEQ ID NO: 91), TPE WPRE (SEQ ID NO: 92), or PTE fn WPRE (SEQ ID NO: 93).

In another aspect, the orientation is S4-S3-S2-S1.

In another aspect, the vector has the sequence PTE CD8 TCR WPRE (SEQ ID NO: 94).

In another aspect, the viral vector is selected from adenoviruses, poxviruses, alphaviruses, arenaviruses, flaviruses, rhabdoviruses, retroviruses, lentiviruses, herpesviruses, paramyxoviruses, or picornaviruses.

In another aspect, the vector is pseudotyped with an envelope protein of a virus selected from the native feline endogenous virus (RD114), a chimeric version of RD114 (RD114TR), gibbon ape leukemia virus (GALV), a chimeric version of GALV (GALV-TR), amphotropic murine leukemia virus (MLV 4070A), baculovirus (GP64), vesicular stomatitis virus (VSV-G), fowl plague virus (FPV), Ebola virus (EboV), baboon retroviral envelope glycoprotein (BaEV), or lymphocytic choriomeningitis virus (LCMV).

In another aspect, the vector is pseudotyped with an envelope protein of vesicular stomatitis virus (VSV-G).

In one aspect, the present disclosure relates to a method of preparing T cells for immunotherapy including isolating T cells from a blood sample of a human subject, activating the isolated T cells in the presence of an aminobisphosphonate, transducing the activated T cells with the vector described herein, and expanding the transduced T cells.

In another aspect, the T cells may be isolated from a leukapheresis human sample.

In another aspect, the aminobisphosphonate may be selected from pamidronic acid, alendronic acid, zoledronic acid, risedronic acid, ibandronic acid, incadronic acid, a salt thereof and/or a hydrate thereof.

In another aspect, the activating may be further in the presence of human recombinant interleukin 2 (IL-2) and human recombinant interleukin 15 (IL-15).

In another aspect, the expanding may be in the presence of IL-2 and IL-15.

In another aspect, the T cells may be γδ T cells.

In another aspect, the first dimer Z1Z2 and the second dimer Y1Y2 are co-expressed on the surface of the expanded T cells.

In another aspect, the present disclosure relates to a population of expanded T cells prepared by the method of the above aspects.

In one aspect, the present disclosure relates to a method of treating a patient who has cancer, comprising administering to the patient a composition comprising the population of expanded T cells described herein, in which the T cells kill cancer cells that present a peptide in a complex with an MHC molecule on the surface, wherein the peptide is selected from any of SEQ ID NO: 98-255, in which the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

In another aspect, the composition further includes an adjuvant.

In another aspect, the adjuvant is selected from one or more of anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and IL-23.

In one aspect, the present disclosure relates to a method of eliciting an immune response in a patient who has cancer, comprising administering to the patient a composition comprising the population of expanded T cells described herein, in which the T cells kill cancer cells that present a peptide in a complex with an MHC molecule on the surface, wherein the peptide is selected from any of SEQ ID NO: 98-255, and in which the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

In another aspect, the immune response comprises a cytotoxic T cell response.

In an aspect, the present disclosure provides for methods of preparing T cells by utilizing a statin in a method described herein. In another aspect, the present disclosure provides for methods of preparing T cells by activating the T cells in the presence of a statin.

In yet another aspect, the present disclosure relates to a method of preparing T cells for immunotherapy, including activating the T cells in the presence of a statin, transducing the activated T cells with the vector of the present disclosure, in which the vector may be pseudotyped with an envelope protein of vesicular stomatitis virus (VSV-G), and expanding the transduced T cells.

In another aspect, the T cells may include αβ T cells, γδ T cells, and/or natural killer T cells.

In another aspect, statin may be selected from atorvastatin, cerivastatin, dalvastatin, fluindostatin, fluvastatin, mevastatin, pravastatin, simvastatin, velostatin, and rosuvastatin.

In an aspect, the present disclosure relates to a method of preparing T cells for immunotherapy including activating the T cells, transducing the activated T cells with the vector of the present disclosure, and expanding the transduced T cells.

In another aspect, the activating may be in the presence of an anti-CD3 antibody and an anti-CD28 antibody.

In another aspect, the expanding may be in the presence of IL-7 and IL-15.

### BRIEF DESCRIPTION OF THE DRAWINGS

FIG. 1 shows a γδ T cell manufacturing process according to one embodiment of the present disclosure. γδ T cell manufacturing may include collecting or obtaining white blood cells or PBMC, e.g., leukapheresis product, depleting αβ T cells from PBMC or leukapheresis product, followed by activation, transduction, and expansion of γδ T cells.

FIG. 2 shows transduction strategies with open reading frames (ORFs) shuffling in accordance with some embodiments of the present disclosure.

FIG. 3 shows lentiviral constructs in accordance with some embodiments of the present disclosure.

FIG. 4 shows lentiviruses pseudotyped with RD114TR used for transducing γδ T cells on Day 3 or Day 6 post activation with zoledronate, IL-2, and IL-15. Transduction efficiency was assessed using antibodies specific to TCR (Vβ8) and CD8 (CD8α) via flow cytometry.

FIG. 5A shows a construct in accordance with an embodiment of the present disclosure.

FIG. 5B shows a construct in accordance with another embodiment of the present disclosure.

FIG. 5C shows a construct in accordance with another embodiment of the present disclosure.

FIG. 5D shows a construct in accordance with another embodiment of the present disclosure.

FIG. 6A shows a construct in accordance with another embodiment of the present disclosure.

FIG. 6B shows a construct in accordance with another embodiment of the present disclosure.

FIG. 7 shows a schematic of constructs in accordance with some embodiments of the present disclosure.

FIG. 8A shows a construct in accordance with an embodiment of the present disclosure.

FIG. 8B shows a construct in accordance with another embodiment of the present disclosure.

FIG. 8C shows a construct in accordance with another embodiment of the present disclosure.

FIG. 8D shows a construct in accordance with another embodiment of the present disclosure.

FIG. 9A shows a schematic of constructs in accordance with some embodiments of the present disclosure.

FIG. 9B shows a schematic of constructs in accordance with some embodiments of the present disclosure.

FIG. 10 shows % CD8+TCR+ γδ T cells transduced with viral vector containing PTE.CD8.TCR.WPRE, PTE.WPRE, PTE.Fn.WPRE, or TPE.WPRE. Non-transduced (NT) cells serve as control.

FIG. 11 shows median fluorescence intensity (MFI) of CD8 and TCR in γδ T cells transduced with viral vector containing PTE.CD8.TCR.WPRE, PTE.WPRE, PTE.Fn.WPRE, or TPE.WPRE. Non-transduced (NT) cells serve as control.

FIG. 12 shows tumor killing activity of γδ T cells obtained from Donor 3 transduced with viral vector containing PTE.CD8.TCR.WPRE, PTE.WPRE, PTE.Fn.WPRE, or TPE.WPRE in a high antigen expressing tumor cell line, e.g., UACC257 (top panel) or in a low antigen expressing tumor cell line, e.g., U2OS (bottom panel), as determined by Incucyte Cytotoxicity Assay. Target only and non-transduced cells serve as controls.

FIGS. 13A-13C show amount of interferon (IFN)-γ secretion by γδ T cells obtained from Donor 3 transduced with viral vector containing PTE.CD8.TCR.WPRE, PTE.WPRE, PTE.Fn.WPRE, or TPE.WPRE in a high antigen expressing tumor cell line, e.g., UACC257 (FIG. 13A), in a low antigen expressing tumor cell line, e.g., U2OS (FIG. 13B), or in antigen-negative tumor cell line, e.g., MCF-7 (FIG. 13C). Non-transduced cells serve as control.

FIG. 14 shows tumor killing activity of γδ T cells obtained from Donor 4 transduced with viral vector containing PTE.CD8.TCR.WPRE, PTE.WPRE, PTE.Fn.WPRE, or TPE.WPRE in a high antigen expressing tumor cell line, e.g., UACC257 (top panel) or in a low antigen expressing tumor cell line, e.g., U2OS (bottom panel), as determined by Incucyte Cytotoxicity Assay. Target only and non-transduced cells serve as controls.

FIGS. 15A-15C show amount of IFN-γ secretion by γδ T cells obtained from Donor 4 transduced with viral vector containing PTE.CD8.TCR.WPRE, PTE.WPRE, PTE.Fn.WPRE, or TPE.WPRE in a high antigen expressing tumor cell line, e.g., UACC257 (FIG. 15A), in a low antigen expressing tumor cell line, e.g., U2OS (FIG. 15B), or in antigen-negative tumor cell line, e.g., MCF-7 (FIG. 15C). Non-transduced cells serve as control.

FIG. 16 shows copy number of viral vector in γδ T cells transduced with viral vector containing PTE.CD8.TCR.WPRE, PTE.WPRE, PTE.Fn.WPRE, or TPE.WPRE. Non-transduced cells serve as control.

FIGS. 17A and 17B show fold expansion of γδ T cells obtained from Donor 3 (FIG. 17A) or Donor 4 (FIG. 17B) transduced with viral vector containing PTE.CD8.TCR.WPRE, PTE.WPRE, PTE.Fn.WPRE, or TPE.WPRE. Non-transduced (NT) cells serve as control.

FIG. 18A shows memory phenotypes of γδ T cells determined by flow cytometry in accordance with some embodiments of the present disclosure.

FIG. 18B shows memory phenotypes of γδ T cells transduced with viral vector containing PTE.CD8.TCR.WPRE, PTE.WPRE, PTE.Fn.WPRE, or TPE.WPRE. Non-transduced (NT) cells serve as control.

FIG. 19 shows comparison of transduction efficiency between γδ T cells transduced with a single lentiviral vector (LV) containing PTE.CD8.TCR.WPRE (panel B (120 μl) and panel C (240 μl)) or transduced with two separate lentiviral vectors: one containing R11KE.WPRE and the other containing CD8, WPRE (panels D and E), with increasing amount of viral vectors, e.g., 120 μl of each R11KE.WPRE and CD8, WPRE (panel D) and 240 μl of each R11KE.WPRE and CD8, WPRE (panel E). Non-transduced (NT) cells serve as control.

FIG. 20 shows enhanced transduction efficiency in γδ T cells transduced with increasing amount of viral vector containing PTE.CD8.TCR.WPRE, e.g., 30 μl, 120 μl, and 240 μl. Non-transduced cells serve as control.

FIG. 21 shows enforced CD8 expression in CD4+ T cells obtained from Donor 5 and Donor 6 using various dilutions of lentiviral vector (LV) expressing 4-in-1 construct of the present disclosure, e.g., LV-PTE.CD8.TCR.WPRE.

FIG. 22 shows detection of TCR expression in CD4+ T cells using various dilutions of LV expressing 4-in-1 construct of the present disclosure, e.g., LV-PTE.CD8.TCR.WPRE.

FIG. 23 shows % target peptide/MHC complex Dextramer203 (Dex203)+ in CD4+ and/or CD8+ T cells obtained from Donor 5 (top panel) and Donor 6 (bottom panel) transduced with 4-in-1 construct of the present disclosure, e.g., LV-PTE.CD8.TCR.WPRE.

FIG. 24 shows Dex203 MFI in CD4+ and/or CD8+ T cells obtained from Donor 5 (top panel) and Donor 6 (bottom panel) transduced with 4-in-1 construct of the present disclosure, e.g., LV-PTE.CD8.TCR.WPRE.

FIG. 25 shows an experimental design for testing functionality of T cells transduced with 4-in-1 construct or TCR-only construct in accordance with one embodiment of the present disclosure.

FIG. 26 shows increased % IFN-γ-positive cells (top panel) and increased IFN-γ MFI (bottom panel) in CD4-CD8α+ T cells obtained from grouped donors transduced with a lentiviral vector containing R11KE.WPRE (LV-TCR) (TCR) or a lentiviral vector containing PTE.CD8.TCR.WPRE (LV-CD8.TCR) (TCR+CD8) followed by co-culturing with high-target expressing UACC257 cells as compared with that co-culturing with non-target expressing MCF7. Non-transduced (NT) cells serve as control. (Effector to target cell ratio=2:1 and Donors grouped N=4).

FIG. 27 shows increased % Granzyme B-positive cells (top panel) and increased Granzyme B MFI (bottom panel) in CD4-CD8α+ T cells obtained from grouped donors transduced with LV-TCR (TCR) or LV-CD8.TCR (TCR+CD8) followed by co-culturing with high-target expressing UACC257 cells as compared with that co-culturing with non-target expressing MCF7. Non-transduced (NT) cells serve as control. (Effector to target cell ratio=2:1 and Donors grouped N=3).

FIG. 28 shows increased % IFN-γ-positive cells (top panel) and increased IFN-γ MFI (bottom panel) in CD4+CD8α+ T cells obtained from grouped donors transduced with LV-CD8.TCR (TCR+CD8) or without transduction (NT) followed by co-culturing with high-target expressing UACC257 cells as compared with that co-culturing with non-target expressing MCF7. (Effector to target cell ratio=2:1 and Donors grouped N=4).

FIG. 29 shows increased % Granzyme B-positive cells (top panel) and increased Granzyme B MFI (bottom panel) in CD4+CD8α+ T cells obtained from grouped donors transduced with LV-CD8.TCR (TCR+CD8) or without transduction (NT) followed by co-culturing with high-target expressing UACC257 cells as compared with that co-culturing with non-target expressing MCF7. (Effector to target cell ratio=2:1 and Donors grouped N=4).

FIG. 30 shows increased % IFN-γ-positive cells (top panel) and increased IFN-γ MFI (bottom panel) in CD3+ T cells obtained from grouped donors transduced with LV-TCR (TCR) or LV-CD8.TCR (TCR+CD8) followed by co-culturing with high-target expressing UACC257 cells as compared with that co-culturing with non-target expressing MCF7. Non-transduced (NT) cells serve as control. (Effector to target cell ratio=2:1 and Donors grouped N=4).

FIG. 31 shows increased % Granzyme B-positive cells (top panel) and increased Granzyme B MFI (bottom panel) in CD3+ T cells obtained from grouped donors transduced with LV-TCR (TCR) or LV-CD8.TCR (TCR+CD8) followed by co-culturing with high-target expressing UACC257 cells as compared with that co-culturing with non-target expressing MCF7. Non-transduced (NT) cells serve as control. (Effector to target cell ratio=2:1 and Donors grouped N=3).

FIG. 32 shows increased IFN-γ secretion in CD3+ T cells obtained from grouped donors transduced with LV-TCR (TCR) or LV-CD8.TCR (TCR+CD8) followed by co-culturing with high-target expressing UACC257 cells as compared with that co-culturing with non-target expressing MCF7. Non-transduced (NT) cells, UACC257 cells, and MCF7 cells serve as control. (Effector to target cell ratio=2:1 and Donors grouped N=4).

FIG. 33 shows increased IFN-γ secretion in CD3+ T cells obtained from individual Donors 5, 6, 7, and 8 transduced with LV-TCR (TCR) or LV-CD8.TCR (TCR+CD8) followed by co-culturing with high-target expressing UACC257 cells as compared with that co-culturing with non-target expressing MCF7. Non-transduced (NT) cells, UACC257 cells only, and MCF7 cells only serve as control. (Effector to target cell ratio=2:1).

FIG. 34 shows % CD25+ cells (top panel), % CD69+ cells (middle panel), and % human low-density lipoprotein receptor (hLDLR)+ cells (bottom panel) in CD3+CD4+ T cells treated with atorvastatin, pravastatin, or rosuvastatin. Pre-activated cells, cells activated without statin or DMSO (control), and DMSO serve as controls.

FIG. 35 shows % CD25+ cells (top panel), % CD69+ cells (middle panel), and % hLDLR+ cells (bottom panel) in CD3+CD8+ T cells treated with atorvastatin, pravastatin, or rosuvastatin. Pre-activated cells, cells activated without statin or DMSO (control), and DMSO serve as controls.

FIG. 36 shows titers of lentiviral vectors in accordance with one embodiment of the present disclosure.

FIG. 37 shows T cell manufacturing process in accordance with one embodiment of the present disclosure.

### DETAILED DESCRIPTION

As used herein, the term “self-cleaving 2A peptide” refers to relatively short peptides (of the order of 20 amino acids long, depending on the virus of origin) acting co-translationally, by preventing the formation of a normal peptide bond between the glycine and last proline, resulting in the ribosome skipping to the next codon, and the nascent peptide cleaving between the Gly and Pro. After cleavage, the short 2A peptide remains fused to the C-terminus of the ‘upstream’ protein, while the proline is added to the N-terminus of the ‘downstream’ protein. Self-cleaving 2A peptide may be selected from porcine teschovirus-1 (P2A), equine rhinitis A virus (E2A), Thosea asigna virus (T2A), foot-and-mouth disease virus (F2A), or any combination thereof (see, e.g., Kim et al., PLOS One 6: e18556, 2011, the content of which including 2A nucleic acid and amino acid sequences are incorporated herein by reference in their entireties). By adding the linker sequences (GSG or SGSG (SEQ ID NO: 8)) before the self-cleaving 2A sequence, this may enable efficient synthesis of biologically active proteins, e.g., TCRs.

As used herein, the term “promoter” refers to a regulatory region of DNA generally located upstream (towards the 5′ region of the sense strand) of a gene that allows transcription of the gene. The promoter contains specific DNA sequences and response elements that are recognized by proteins known as transcription factors. These factors bind to the promoter sequences, recruiting RNA polymerase, the enzyme that synthesizes the RNA from the coding region of the gene. For example, the promoter sequence used herein may be selected from cytomegalovirus (CMV) promoter, phosphoglycerate kinase (PGK) promoter, myelin basic protein (MBP) promoter, glial fibrillary acidic protein (GFAP) promoter, modified MoMuLV LTR containing myeloproliferative sarcoma virus enhancer (MNDU3), Ubiqitin C promoter, EF-1 alpha promoter, or Murine Stem Cell Virus (MSCV) promoter.

The term “constitutive promoter” as used herein may include a regulatory sequence that directs transcription of a gene in most cells or tissues at most times. In some non-limiting embodiments, the constitutive promoter may be selected from the group consisting of a MSCV promoter, a Ubiqitin C (Ubc) promoter, a CMV promoter, an EF-1 alpha promoter, a PGK promoter, a beta-actin promoter, and a ROSA26 promoter.

In some embodiments, the promoter may be an inducible promoter. The activity of an inducible promoter may increase or decrease in response to a signal. For example, an inducible promoter may promote transcription in response to the presence of a signal, such as T cell activation or isopropyl β-D-1-thiogalactopyranoside (IPTG). An inducible promoter may promote transcription in response to the absence of a signal, such as phosphate. In either of these scenarios, the amount of transcription may or may not be proportional to the amount of signal, or the deficiency thereof. Examples of inducible promoters suitable for prokaryotic host cells may include, without limitation, NFAT, CD69, lac, tac, trc, trp, pho, recA, tetA, nar, phage PL, cspA, T7, and PBAD promoters (see Terpe K. 2006 Appl. Microbiol. Biotechnol. 72:211; the content of which is incorporated by reference in its entirety).

In some embodiments, the inducible promoter may include a nuclear factor of activated T cells (NFAT)/AP1 transcriptional response element (TRE). Upon recognition of the cognate peptide/MHC1 complex, NFAT may undergo Ca2+ dependent translocation to the nucleus, where it promotes transcription of genes that harbor an NFAT TRE. Suitable NFAT TREs are well-known in the art and include the human IL2 promoter NFAT TRE (Macian et al (2001) Oncogene, 2001 Apr. 30; 20(19): 2476-89). Zhang et al. (“Tumor-Infiltrating Lymphocytes Genetically Engineered with an Inducible Gene Encoding Interleukin-12 for the Immunotherapy of Metastatic Melanoma,” Clin. Cancer Res. 21:2278-2288, 2015) describes the use of human tumor-infiltrating lymphocytes (TILs) genetically engineered to secrete single-chain IL12, whose expression is driven by an inducible NFAT promoter, in a clinical trial. The contents of these cited references are incorporated by reference in their entireties.

In some embodiments, the inducible promoter may include a CD69 promoter, e.g., as disclosed in U.S. Pat. No. 5,759,805; the content of which is incorporated by reference in its entirety. CD69 may be among the earliest of these newly synthesized cell-surface activation molecules induced on activated T cells. CD69 expression can be observed within 60 minutes of T-cell stimulation, but may be absent on resting cells. CD69 expression may be also inducible on thymocytes, B cells, natural killer (NK) cells and neutrophils. Four non-coding regions referred to as CNS1-4 located within 50 kb upstream of the mouse CD69 promoter may contribute to the developmental and temporal control of CD69 activation in T- and B-cells. CNS2 region may function as a potent enhancer. Kulemzin et al. (“Design and analysis of stably integrated reporters for inducible transgene expression in human T cells and chimeric antigen receptor (CAR) NK cell lines,” BMC Medical Genomics 2019, 12 (Suppl 2): 44, 88-95; the content of which is incorporated by reference in its entirety) describes, in the context of primary T cells, activation-inducible CD69 promoter variant provides the highest fold induction. This promoter therefore can be used for expressing proteins in the activated, but not resting human T or CAR T cells.

In some embodiments, the inducible promoter may be an IPTG-inducible promoter. An IPTG-inducible promoter may refer to any polynucleotide sequence that promotes transcription in a manner responsive to IPTG or any other lactose derivative that can promote transcription from the lac operon (e.g., allolactose). Many examples of IPTG-inducible promoters are known in the art, including, without limitation, tac (e.g., tacI, tacII, etc.) promoters, lac promoters, and derivatives thereof (e.g., lacUV5, taclac, and so forth).

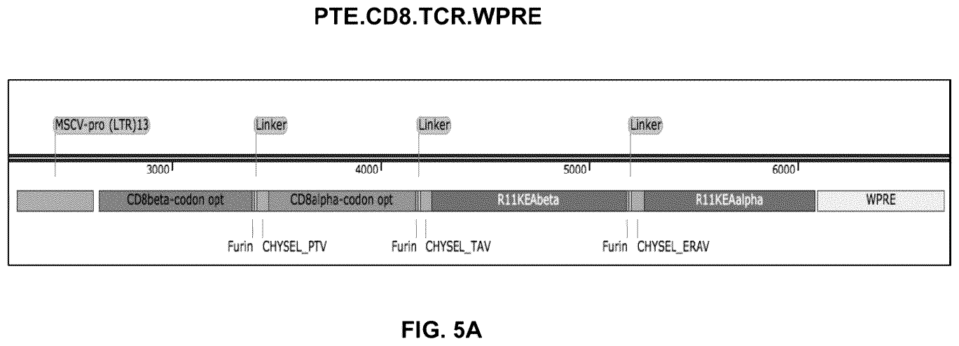

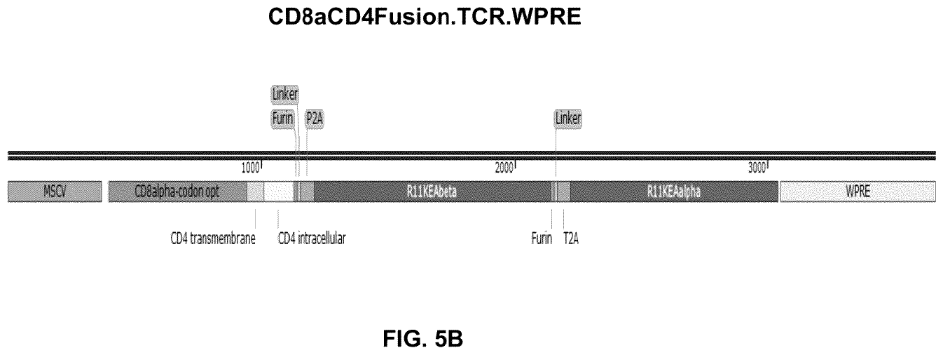

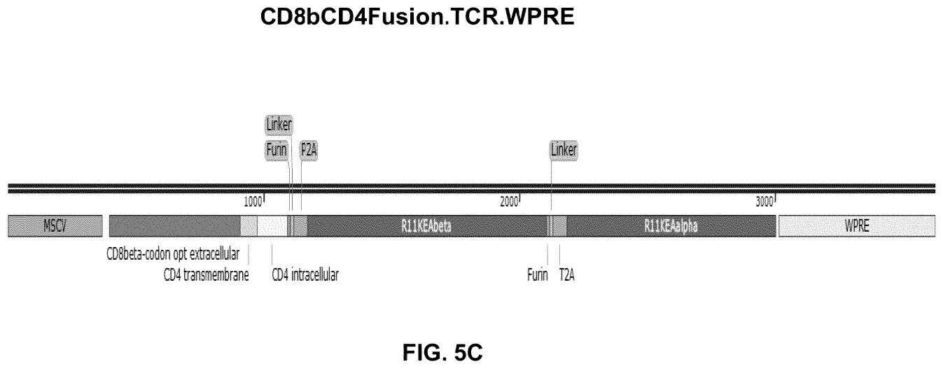

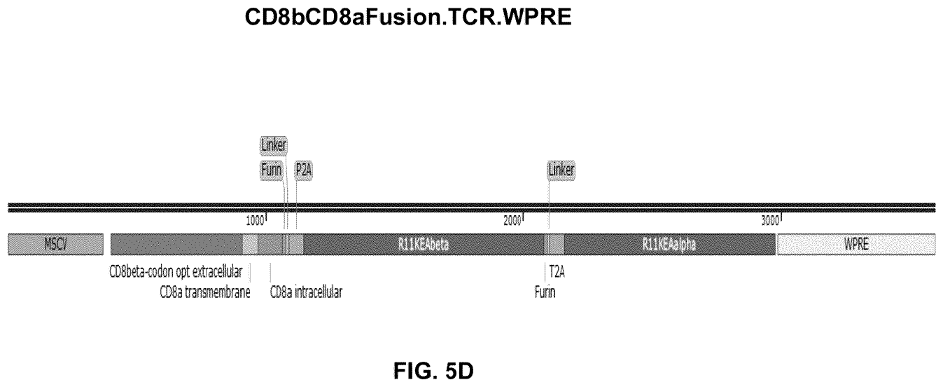

In an aspect, expression of a 4-in-1 viral vector, e.g., lentiviral vector, containing sequences encoding CD8 alpha chains, CD8 beta chain, TCR alpha chain, and TCR beta chain may be driven by a constitutive or inducible promoter. For example, FIG. 5A shows a 4-in-1 viral vector containing PTE CD8 TCR WPRE (SEQ ID NO: 94) having codon-optimized sequences encoding CD8 alpha (SEQ ID NO: 12) and CD8 beta (SEQ ID NO: 13) located upstream from sequences encoding a TCR, e.g., TCR R11KE alpha chain (SEQ ID NO: 13) and R11KE beta chain (SEQ ID NO: 14) and driven by a constitutive MSCV promoter (SEQ ID NO: 1). The same coding sequences described above can also be driven by an inducible promote, e.g., NFAT, CD69, or IPTG promoter.

In another aspect, expression of a 3-in-1 viral vector containing sequences encoding a fusion protein, TCR alpha chain, and TCR beta chain may be driven by a constitutive or inducible promoter. For example, FIG. 5B shows a viral vector containing CD8aCD4Fusion.TCR WPRE (SEQ ID NO: 256) having codon-optimized sequence encoding a fusion protein, in which CD8a extracellular domain is fused with CD4 transmembrane domain and CD4 intracellular domain, and sequences encoding TCR R11KE alpha chain (SEQ ID NO: 13) and R11KE beta chain (SEQ ID NO: 14) driven by MSCV promoter (SEQ ID NO: 1). FIG. 5C shows a viral vector containing CD8bCD4Fusion.TCR WPRE (SEQ ID NO: 257) having codon-optimized sequence encoding a fusion protein, in which CD8β extracellular domain is fused with CD4 transmembrane domain and CD4 intracellular domain, and sequences encoding TCR R11KE alpha chain (SEQ ID NO: 13) and R11KE beta chain (SEQ ID NO: 14) driven by MSCV promoter (SEQ ID NO: 1). FIG. 5D shows a viral vector containing CD8bCD8aFusion.TCR WPRE (SEQ ID NO: 258) having sequences encoding a fusion protein, in which CD8β extracellular domain is fused with CD8α transmembrane domain and CD8α intracellular domain, and sequences encoding TCR R11KE alpha chain (SEQ ID NO: 13) and R11KE beta chain (SEQ ID NO: 14) driven by MSCV promoter (SEQ ID NO: 1). The same coding sequences described above can also be driven by an inducible promote, e.g., NFAT, CD69, or IPTG promoter.

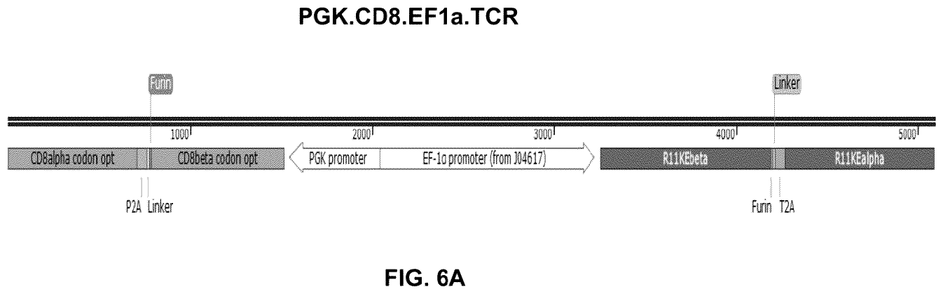

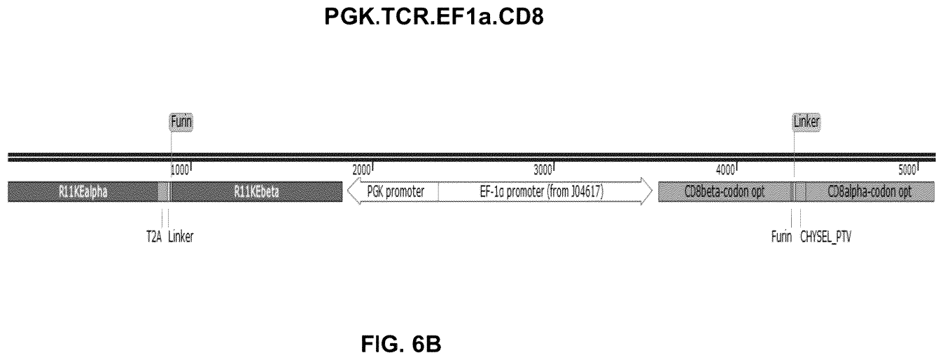

In an aspect, expression of a 4-in-1 viral vector of the present disclosure may be driven by bidirectional constitutive and/or inducible promoters. For example, FIG. 6A shows a 4-in-1 viral vector containing PGK.CD8.EF1a.TCR (SEQ ID NO: 259) having codon-optimized sequences encoding CD8 alpha chain and CD8 beta chain located upstream from sequences encoding TCR R11KE alpha chain and R11KE beta chain, in which the sequences encoding CD8 alpha chain and CD8 beta chain and the sequences encoding TCR R11KE alpha chain and R11KE beta chain may be separated by bidirectional promoters, e.g., PGK promoter and EF-1 alpha promoter. PGK promoter may be positioned at the 3′ end of the codon-optimized sequences encoding CD8 alpha chain and CD8 beta chain to drive the expression of CD8 alpha chain and CD8 beta chain. EF-1 alpha promoter may be positioned at the 5′ end of the sequences encoding TCR R11KE alpha chain and R11KE beta chain to drive the expression of TCR R11KE alpha chain and R11KE beta chain.

FIG. 6B shows another 4-in-1 viral vector containing PGK.TCR.EF1a.CD8 (SEQ ID NO: 260) having sequences encoding TCR R11KE alpha chain and R11KE beta chain located upstream from codon-optimized sequences encoding CD8 alpha chain and CD8 beta chain, in which the sequences encoding TCR R11KE alpha chain and R11KE beta chain and the sequences encoding CD8 alpha chain and CD8 beta chain may be separated by bidirectional promoters, e.g., PGK promoter and EF-1 alpha promoter. PGK promoter may be positioned at the 3′ end of the sequences encoding TCR R11KE alpha chain and R11KE beta chain to drive the expression of TCR R11KE alpha chain and R11KE beta chain. EF-1 alpha promoter may be positioned at the 5′ end of the codon-optimized sequences encoding CD8 alpha chain and CD8 beta chain to drive the expression of CD8 alpha chain and CD8 beta chain.

Some embodiments of the present disclosure may include viral vectors containing sequences encoding TCR alpha chain and TCR beta chain and sequences encoding other proteins, such as cytokines (including, but not limited to, IL-1, IL-2, IL-6, IL-7, IL-10, IL-12, IL-15, IL-18, and IL-21), IL-15/IL-15 receptor (IL-15R) fusion protein, dominant-negative TGF beta receptor (DN TGFbRII), and/or extracellular domain of a transforming growth factor-beta receptor. In some embodiments, these coding sequences may be driven by a promotor or bidirectional promoters.

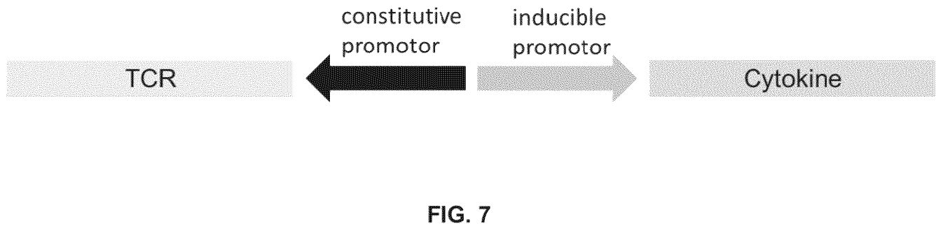

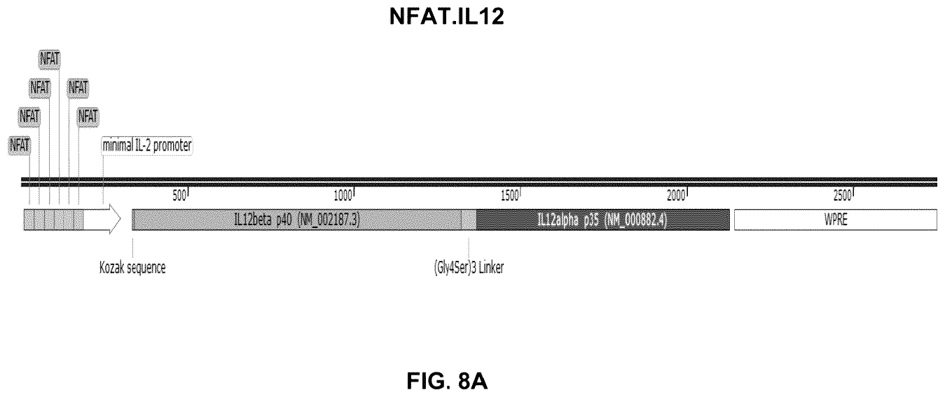

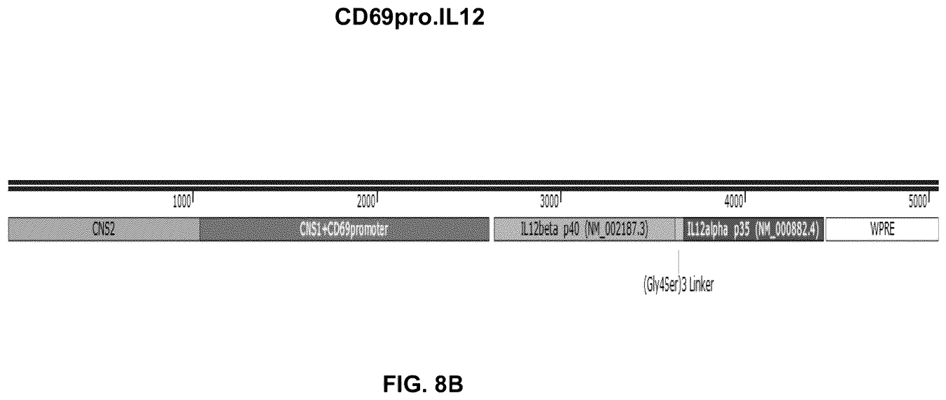

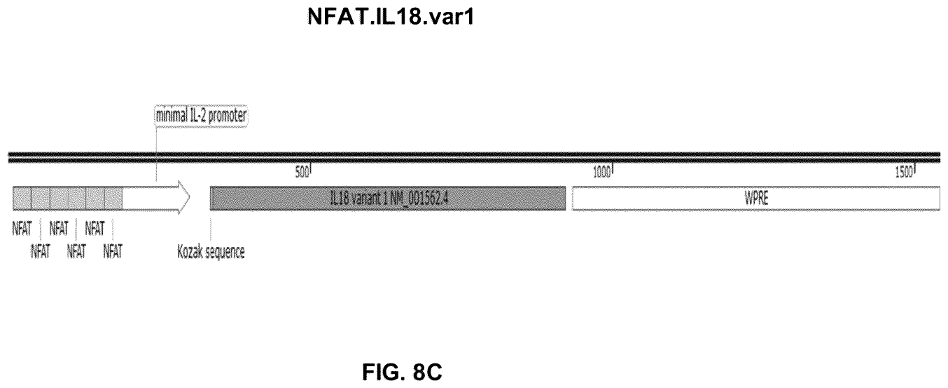

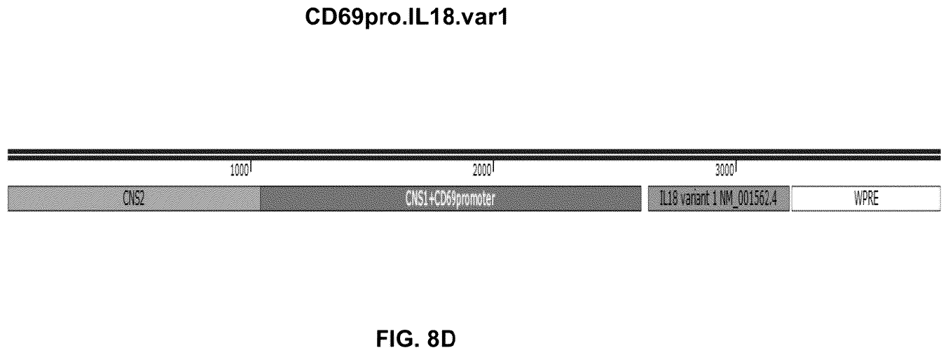

FIG. 7 shows a viral vector containing sequences encoding TCR alpha chain and TCR beta chain located upstream from sequences encoding cytokines, in which the sequences encoding TCR alpha chain and TCR beta chain and sequences encoding cytokines may be separated by bidirectional promoters. Bidirectional promoters may be arranged in, from 5′ to 3′ direction, constitutive-constitutive, constitutive-inducible, inducible-constitutive, or inducible-inducible orientation. For example, a constitutive promoter, e.g., MSCV, PGK, or EF1 alpha promoter, may be positioned at the 3′ end of the sequences encoding TCR alpha chain and TCR beta chain to drive the expression of TCR alpha chain and TCR beta chain. An inducible promote, e.g., NFAT, CD69, or IPTG promoter, may be positioned at the 5′ end of the sequences encoding cytokines to drive the expression of cytokines. FIG. 8A shows an inducible NFAT promoter with minimal IL-2 promoter positioned at the 5′ end of the sequences encoding IL-12, e.g., IL-12alpha (p35)/IL-12beta (p40) fusion protein (SEQ ID NO: 261) to drive the expression of 12alpha (p35)/IL-12beta (p40) fusion protein in a viral vector shown in FIG. 7. FIG. 8B shows an inducible CD69 promoter with CNS1 and CNS2 enhancer elements positioned at the 5′ end of the sequences encoding IL-12, e.g., IL-12alpha (p35)/IL-12beta (p40) fusion protein (SEQ ID NO: 262) to drive the expression of 12alpha (p35)/IL-12beta (p40) fusion protein in a viral vector shown in FIG. 7. FIG. 8C shows an inducible NFAT promoter with minimal IL-2 promoter positioned at the 5′ end of the sequences encoding IL-18, e.g., IL-18 variant 1 (SEQ ID NO: 263) to drive the expression of IL-18 variant 1 in a viral vector shown in FIG. 7. FIG. 8D shows an inducible CD69 promoter with CNS1 and CNS2 enhancer elements positioned at the 5′ end of the sequences encoding IL-18, e.g., IL-18 variant 1 (SEQ ID NO: 264) to drive the expression of IL-18 variant 1 in a viral vector shown in FIG. 7.

In an aspect, the disclosure provides for 4-in-1 construct with a 5′ end to 3′ end direction orientation of CD8β-CD8α-TCRβ-TCRα. In another aspect, the disclosure provides for 4-in-1 construct with a 5′ end to 3′ end direction orientation of CD8β-CD8α-TCRα-TCRβ. In another aspect, the disclosure provides for 4-in-1 construct with a 5′ end to 3′ end direction orientation of CD8α-CD8β-TCRβ-TCRα. In another aspect, the disclosure provides for 4-in-1 construct with a 5′ end to 3′ end direction orientation of CD8α-CD8β-TCRα-TCRβ.

In an aspect, the disclosure provides for 4-in-1 construct with a 5′ end to 3′ end direction orientation does not include TCRβ-TCRα-CD8α-CD8β. In another aspect, the disclosure provides for 4-in-1 construct with a 5′ end to 3′ end direction orientation does not include TCRβ-TCRα-CD8β-CD8α. In another aspect, the disclosure provides for 4-in-1 construct with a 5′ end to 3′ end direction orientation does not include TCRα-TCRβ-CD8α-CD8β. In another aspect, the disclosure provides for 4-in-1 construct with a 5′ end to 3′ end direction orientation does not include TCRα-TCRβ-CD8β-CD8α.

In an aspect, the disclosure provides for 4-in-1 construct with a 5′ end to 3′ end direction orientation of CD8β-CD8α-TCRβ-TCRα. In a non-limiting aspect, the disclosure provides for 4-in-1 construct with a 5′ end to 3′ end direction orientation does not include TCRβ-TCRα-CD8α-CD8β.

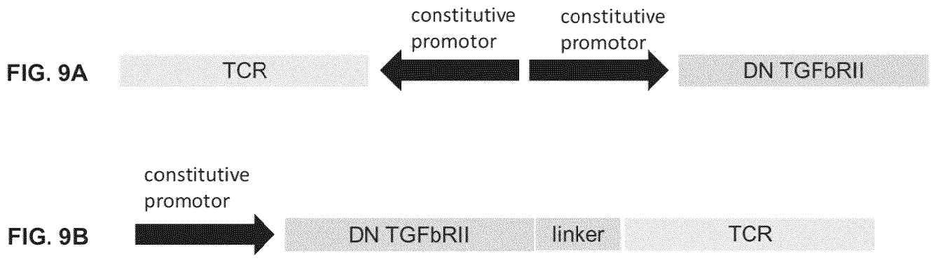

In some embodiments, viral vectors of the present disclosure may contain sequences encoding TCR alpha chain and TCR beta chain and sequences encoding a TGF-beta inhibitors, e.g., dominant-negative TGF beta receptor (DN TGFbRII), and/or extracellular domain of a transforming growth factor-beta receptor. FIG. 9A shows a viral vector containing sequences encoding TCR alpha chain and TCR beta chains located upstream from sequences encoding DN TGFbRII, in which the sequences encoding TCR alpha chain and TCR beta chains and the sequences encoding DN TGFbRII may be separated by bidirectional promoters. For example, FIG. 9A shows a constitutive promoter, e.g., MSCV, Ubc, CMV, EF-1 alpha, and PGK promoter, may be positioned at the 3′ end of the sequences encoding TCR alpha chain and TCR beta chain to drive the expression of TCR alpha chain and TCR beta chain; and another constitutive promoter may be positioned at the 5′ end of the sequences encoding DN TGFbRII to drive the expression of DN TGFbRII.

Alternatively, FIG. 9B shows a viral vector containing a constitutive promoter, e.g., MSCV, Ubc, CMV, EF-1 alpha, and PGK promoter, positioned at the 5′ end of sequences encoding DN TGFbRII located upstream from sequences encoding TCR alpha chain and TCR beta chain to drive the expression of DN TGFbRII, TCR alpha chain, and TCR beta chain. The same coding sequences described above can also be driven by an inducible promote, e.g., NFAT, CD69, or IPTG promoter.

As used herein, the term “cistron” refers to a section of the DNA molecule that specifies the formation of one polypeptide chain, i.e. coding for one polypeptide chain. For example, “bi-cistron” refers to two sections of the DNA molecule that specify the formation of two polypeptide chains, i.e. coding for two polypeptide chains; “tri-cistron” refers to three sections of the DNA molecule that specify the formation of three polypeptide chains, i.e. coding for three polypeptide chains; etc.

As used herein, the term “multi-cistronic RNA” or “multi-cistronic RNA” refers to an RNA that contains the genetic information to translate to several proteins. In contrast, a mono-cistronic RNA contains the genetic information to translate only a single protein. In the context of the present disclosure, the multi-cistronic RNA transcribed from the lentivirus in the Examples 2-4 may be translated into four proteins (4-in-1): TCRα chain, TCRβ chain, CD8α chain, and CD8β chain; or translated to two proteins (2-in-1): TCRα chain and TCRβ chain or CD8α chain and CD8β chain.

As used herein, the term “arranged in tandem” refers to the arrangement of the genes contiguously, one following or behind the other, in a single file on a nucleic acid sequence. The genes are ligated together contiguously on a nucleic acid sequence, with the coding strands (sense strands) of each gene ligated together on a nucleic acid sequence.

As used herein, the term “sense strand” refers to the DNA strand of a gene that is translated or translatable into protein. When a gene is oriented in the “sense direction” with respect to the promoter in a nucleic acid sequence, the “sense strand” is located at the 5′ end downstream of the promoter, wherein the first codon of the nucleic acid encoding the protein is proximal to the promoter and the last codon is distal from the promoter.

As used herein, the term “viral vector” refers to a nucleic acid vector construct that includes at least one element of viral origin and has the capacity to be packaged into a viral vector particle, and encodes at least an exogenous nucleic acid. The vector and/or particle can be utilized for the purpose of transferring any nucleic acids into cells either in vitro or in vivo. Numerous forms of viral vectors are known in the art. The term “virion” is used to refer to a single infective viral particle. “Viral vector”, “viral vector particle” and “viral particle” also refer to a complete virus particle with its DNA or RNA core and protein coat as it exists outside the cell. For example, a viral vector may be selected from adenoviruses, poxviruses, alphaviruses, arenaviruses, flaviruses, rhabdoviruses, retroviruses, lentiviruses, herpesviruses, paramyxoviruses, or picornaviruses.

The terms “T cell” or “T lymphocyte” are art-recognized and are intended to include thymocytes, naïve T lymphocytes, immature T lymphocytes, mature T lymphocytes, resting T lymphocytes, or activated T lymphocytes. Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4−CD8− T cell, natural killer T cell, T cells expressing αβ TCR (αβ T cells), T cells expressing γδ TCR (γδ T cells), or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

The term “statin,” “vastatin,” or as used interchangeably herein “3-hydroxy-3-methylglutaryl coenzyme A (HMG-COA) reductase inhibitor” refers to a pharmaceutical agent which inhibits the enzyme 3-hydroxy-3-methylglutaryl-coenzyme A (HMG-COA) reductase. This enzyme is involved in the conversion of HMG-COA to mevalonate, which is one of the steps in cholesterol biosynthesis. Such inhibition is readily determined according to standard assays well known to those skilled in the art.

Preferred statins which may be used in accordance with this present disclosure include atorvastatin, disclosed in U.S. Pat. No. 4,681,893; atorvastatin calcium, disclosed in U.S. Pat. No. 5,273,995; cerivastatin, disclosed in U.S. Pat. No. 5,502,199; dalvastatin, disclosed in U.S. Pat. No. 5,316,765; fluindostatin, disclosed in U.S. Pat. No. 4,915,954; fluvastatin, disclosed in U.S. Pat. No. 4,739,073; lovastatin, disclosed in U.S. Pat. No. 4,231,938; mevastatin, disclosed in U.S. Pat. No. 3,983,140; pravastatin, disclosed in U.S. Pat. No. 4,346,227; simvastatin, disclosed in U.S. Pat. No. 4,444,784; velostatin, disclosed in U.S. Pat. Nos. 4,448,784 and 4,450,171; and rosuvastatin, disclosed in U.S. Pat. Nos. 6,858,618 and 7,511,140, the contents of each of these references are herein incorporated by reference in their entireties. Representative 3-hydroxy-3-methylglutaryl coenzyme A reductase inhibitors may include atorvastatin, atorvastatin calcium, also known as Liptor®, lovastatin, also known as Mevacor®, pravastatin, also known as Pravachol®, simvastatin, also known as Zocor®, and rosuvastatin.

In one aspect, the present disclosure relates to activation, transduction, and/or expansion of T cells, e.g., tumor-infiltrating lymphocytes, CD8+ T cells, CD4+ T cells, and γδ T cells, that may be used for transgene expression. In another aspect, the disclosure relates to activation, transduction, and expansion of γδ T cells while depleting α- and/or β-TCR positive cells.

In an aspect, γδ T cells may be isolated from a complex sample that is cultured in vitro. In another aspect, whole PBMC population, without prior depletion of specific cell populations, such as monocytes, αβ T-cells, B-cells, and NK cells, can be activated and expanded. In another aspect, enriched γδ T cell populations can be generated prior to their specific activation and expansion. In another aspect, activation and expansion of γδ T cells may be performed without the presence of native or engineered APCs. In another aspects, isolation and expansion of γδ T cells from tumor specimens can be performed using immobilized γδ T cell mitogens, including antibodies specific to γδ TCR, and other γδ TCR activating agents, including lectins. In another aspect, isolation and expansion of γδ T cells from tumor specimens can be performed in the absence of γδ T cell mitogens, including antibodies specific to γδ TCR, and other γδ TCR activating agents, including lectins.

In an aspect, γδ T cells are isolated from leukapheresis of a subject, for example, a human subject. In another aspect, γδ T cells are not isolated from peripheral blood mononuclear cells (PBMC).

In an aspect, the isolated γδ T cells can rapidly expand in response to contact with one or more antigens. Some γδ T cells, such as Vγ9Vδ2+ T cells, can rapidly expand in vitro in response to contact with some antigens, like prenyl-pyrophosphates, alkyl amines, and metabolites or microbial extracts during tissue culture. Stimulated γδ T-cells can exhibit numerous antigen-presentation, co-stimulation, and adhesion molecules that can facilitate the isolation of γδ T-cells from a complex sample. γδ T cells within a complex sample can be stimulated in vitro with at least one antigen for 1 day, 2 days, 3 days, 4 days, 5 days, 6 days, 7 days, or another suitable period of time. Stimulation of γδ T cells with a suitable antigen can expand γδ T cell population in vitro.

Non-limiting examples of antigens that may be used to stimulate the expansion of γδ T cells from a complex sample in vitro may include, prenyl-pyrophosphates, such as isopentenyl pyrophosphate (IPP), alkyl-amines, metabolites of human microbial pathogens, metabolites of commensal bacteria, methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates.

Activation and expansion of γδ T cells can be performed using activation and co-stimulatory agents described herein to trigger specific γδ T cell proliferation and persistence populations. In an aspect, activation and expansion of γδ T-cells from different cultures can achieve distinct clonal or mixed polyclonal population subsets. In another aspect, different agonist agents can be used to identify agents that provide specific γδ activating signals. In another aspect, agents that provide specific γδ activating signals can be different monoclonal antibodies (MAbs) directed against the γδ TCRs. In another aspect, companion co-stimulatory agents to assist in triggering specific γδ T cell proliferation without induction of cell energy and apoptosis can be used. These co-stimulatory agents can include ligands binding to receptors expressed on γδ cells, such as NKG2D, CD161, CD70, JAML, DNAX accessory molecule-1 (DNAM-1), ICOS, CD27, CD137, CD30, HVEM, SLAM, CD122, DAP, and CD28. In another aspect, co-stimulatory agents can be antibodies specific to unique epitopes on CD2 and CD3 molecules. CD2 and CD3 can have different conformation structures when expressed on αβ or γδ T-cells. In another aspect, specific antibodies to CD3 and CD2 can lead to distinct activation of γδ T cells.

A population of γδ T-cells may be expanded ex vivo prior to engineering of the γδ T-cells. Non-limiting example of reagents that can be used to facilitate the expansion of a γδ T-cell population in vitro may include anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), Les culinaris Agglutinin (LCA), Pisum sativum Agglutinin (PSA), Helix pomatia agglutinin (HPA), Vicia graminea Lectin (VGA), or another suitable mitogen capable of stimulating T-cell proliferation.

The ability of γδ T cells to recognize a broad spectrum of antigens can be enhanced by genetic engineering of the γδ T cells. In an aspect, γδ T cells can be engineered to provide a universal allogeneic therapy that recognizes an antigen of choice in vivo. Genetic engineering of the γδ T-cells may include stably integrating a construct expressing a tumor recognition moiety, such as αβ TCR, γδ TCR, chimeric antigen receptor (CAR), which combines both antigen-binding and T-cell activating functions into a single receptor, an antigen binding fragment thereof, or a lymphocyte activation domain into the genome of the isolated γδ T-cell(s), a cytokine (for example, IL-15, IL-12, IL-2. IL-7. IL-21, IL-18, IL-19, IL-33, IL-4, IL-9, IL-23, or IL1B) to enhance T-cell proliferation, survival, and function ex vivo and in vivo. Genetic engineering of the isolated γδ T-cell may also include deleting or disrupting gene expression from one or more endogenous genes in the genome of the isolated γδ T-cells, such as the MHC locus (loci).

Engineered γδ T-cells may be generated with various methods. For example, a polynucleotide encoding an expression cassette that comprises a tumor recognition, or another type of recognition moiety, can be stably introduced into the γδ T-cell by a transposon/transposase system or a viral-based gene transfer system, such as a lentiviral or a retroviral system, or another suitable method, such as transfection, electroporation, transduction, lipofection, calcium phosphate (CaPO₄), nanoengineered substances, such as Ormosil, viral delivery methods, including adenoviruses, retroviruses, lentiviruses, adeno-associated viruses, or another suitable method. A number of viral methods have been used for human gene therapy, such as the methods described in WO 1993020221, the content of which is incorporated herein in its entirety. Non-limiting examples of viral methods that can be used to engineer γδ T cells may include γ-retroviral, adenoviral, lentiviral, herpes simplex virus, vaccinia virus, pox virus, or adeno-virus associated viral methods.

In an aspect, constructs and vectors described herein are used with the methodology described in U.S. Ser. No. 16/200,308, filed on Nov. 26, 2018, the contents of which are incorporated by reference in their entirety.

In an aspect, viruses refer to natural occurring viruses as well as artificial viruses. Viruses in accordance to some embodiments of the present disclosure may be either an enveloped or non-enveloped virus. Parvoviruses (such as AAVs) are examples of non-enveloped viruses. In a preferred embodiment, the viruses may be enveloped viruses. In preferred embodiments, the viruses may be retroviruses and in particular lentiviruses. Viral envelope proteins that can promote viral infection of eukaryotic cells may include HIV-1 derived lentiviral vectors (LVs) pseudotyped with envelope glycoproteins (GPs) from the vesicular stomatitis virus (VSV-G), the modified feline endogenous retrovirus (RD114TR) (SEQ ID NO: 97), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; the content of which is incorporated herein by reference), RD114 env, chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235:1269-1276; the content of which is incorporated herein by reference), baculovirus GP64 env (such as described in Wang et al. J. Virol. 81:10869-10878, 2007; the content of which is incorporated herein by reference), or GALV env (such as described in Merten et al., J. Virol. 79:834-840, 2005; the content of which is incorporated herein by reference), or derivatives thereof.

Embodiments of the present disclosure are based on the discovery that a single lentiviral cassette can be used to create a single lentiviral vector, expressing at least four individual monomer proteins of two distinct dimers from a single multi-cistronic mRNA so as to co-express the dimers on the cell surface. For example, the integration of a single copy of the lentiviral vector was sufficient to transform γδ T cells to co-express TCRαβ and CD8αβ.

In one aspect, the present disclosure relates to vectors containing a multi-cistronic cassette within a single vector capable of expressing more than one, more than two, more than three, more than four genes, more than five genes, or more than six genes, in which the polypeptides encoded by these genes may interact with one another, or may form dimers. The dimers may be homodimers, i.e., two identical proteins forming a dimer, or heterodimers, i.e., two structurally different proteins forming a dimer.

In one aspect, a lentiviral vector may contain a first nucleotide sequence S1 encoding a protein Z1, a second nucleotide sequence S2 encoding a protein Z2, a third nucleotide sequence S3 encoding a protein Y1, and a fourth nucleotide sequence S4 encoding a protein Y2, in which Z1 and Z2 form a first dimer and Y1 and Y2 form a second dimer, in which the first dimer Z1Z2 is different from the second dimer Y1Y2.

In one aspect, a first lentiviral vector may contain a bi-cistronic cassette (2-in-1) encoding a dimer Z1Z2, and a second lentiviral vector may contain a bi-cistronic cassette (2-in-1) encoding a dimer Y1Y2. In the 2-in-1 vectors, S1 and S2 may be arranged in tandem in a 5′ to 3′ orientation of S1-S2 or S2-S1. Likewise, In the 2-in-1 vectors, S3 and S4 may be arranged in tandem in a 5′ to 3′ orientation of S3-S4 or S4-S3. Z1 and Z2 or Y1 and Y2 may be separated by one or more self-cleaving 2A peptides.

In another aspect, a single lentiviral vector (4-in-1) may encode both distinct dimers Z1Z2 and Y1Y2, in which Z1, Z2, Y1, and Y2 may be separated by one or more self-cleaving 2A peptides. For example, the S1, S2, S3, and S4 may be arranged in tandem in a 5′ to 3′ orientation selected from S1-S2-S3-S4, S1-S2-S4-S3, S1-S3-S2-S4, S1-S3-S4-S2, S1-S4-S3-S2, S1-S4-S2-S3, S2-S1-S3-S4, S2-S1-S4-S3, S2-S3-S1-S4, S2-S3-S4-S1, S2-S4-S3-S1, S2-S4-S1-S3, S3-S1-S2-S4, S3-S1-S4-S2, S3-S2-S1-S4, S3-S2-S4-S1, S3-S4-S1-S2, S3-S4-S2-S1, S4-S1-S2-S3, S4-S1-S3-S2, S4-S2-S1-S3, S4-S2-S3-S1, S4-S3-S1-S2, or S4-S3-S2-S1.

In an aspect, the dimer Z1Z2 may be TCRs having a TCRα chain and a TCRβ chain.

In an aspect, TCRs and antigen binding proteins that are capable of use with the constructs, methods and embodiments described herein include, for example, those listed in Table 2 (SEQ ID NOS: 13-90) and those TCRs and antigen binding proteins described in U.S. Publication 20170267738, U.S. Publication 20170312350, U.S. Publication 20180051080, U.S. Publication 20180164315, U.S. Publication 20180161396, U.S. Publication 20180162922, U.S. Publication 20180273602, U.S. Publication 20190016801, U.S. Publication 20190002556, U.S. Publication 20190135914, U.S. Pat. Nos. 10,538,573, 10,626,160, U.S. Publication 20190321478, U.S. Publication 20190256572, U.S. Pat. Nos. 10,550,182, 10,526,407, U.S. Publication 20190284276, U.S. Publication 20190016802, and U.S. Pat. No. 10,583,573, the contents of each of these publications and sequence listings described therein are herein incorporated by reference in their entireties.

In another aspect, the dimer Z1Z2 may be TCRα chain and TCRβ chain selected from R11KEA (SEQ ID NO: 13 and 14), R20P1H7 (SEQ ID NO: 15 and 16), R7P1D5 (SEQ ID NO: 17 and 18), R10P2G12 (SEQ ID NO: 19 and 20), R10P1A7 (SEQ ID NO: 21 and 22), R4P1D10 (SEQ ID NO: 23 and 24), R4P3F9 (SEQ ID NO: 25 and 26), R4P3H3 (SEQ ID NO: 27 and 28), R36P3F9 (SEQ ID NO: 29 and 30), R52P2G11 (SEQ ID NO: 31 and 32), R53P2A9 (SEQ ID NO: 33 and 34), R26P1A9 (SEQ ID NO: 35 and 36), R26P2A6 (SEQ ID NO: 37 and 38), R26P3H1 (SEQ ID NO: 39 and 40), R35P3A4 (SEQ ID NO: 41 and 42), R37P1C9 (SEQ ID NO: 43 and 44), R37P1H1 (SEQ ID NO: 45 and 46), R42P3A9 (SEQ ID NO: 47 and 48), R43P3F2 (SEQ ID NO: 49 and 50), R43P3G5 (SEQ ID NO: 51 and 52), R59P2E7 (SEQ ID NO: 53 and 54), R11P3D3 (SEQ ID NO: 55 and 56), R16P1C10 (SEQ ID NO: 57 and 58), R16P1E8 (SEQ ID NO: 59 and 60), R17P1A9 (SEQ ID NO: 61 and 62), R17P1D7 (SEQ ID NO: 63 and 64), R17P1G3 (SEQ ID NO: 65 and 66), R17P2B6 (SEQ ID NO: 67 and 68), R11P3D3KE (SEQ ID NO: 69 and 70), R39P1C12 (SEQ ID NO: 71 and 72), R39P1F5 (SEQ ID NO: 73 and 74), R40P1C2 (SEQ ID NO: 75 and 76), R41P3E6 (SEQ ID NO: 77 and 78), R43P3G4 (SEQ ID NO: 79 and 80), R44P3B3 (SEQ ID NO: 81 and 82), R44P3E7 (SEQ ID NO: 83 and 84), R49P2B7 (SEQ ID NO: 85 and 86), R55P1G7 (SEQ ID NO: 87 and 88), or R59P2A7 (SEQ ID NO: 89 and 90). In an aspect, the sequences exhibit at least about 90%, at least about 95%, or at least about 98% to any of SEQ ID NO: 13-90.

Table 1 shows examples of the peptides to which TCRs bind when the peptide is in a complex with an MHC molecule.

TABLE 1

| | Peptide (name/ |
|---|---|
| TCR name | sequence/SEQ ID NO:) |
| R20P1H7, R7P1D5, R10P2G12 | MAG-003 (KVLEHVVRV) |
| | (SEQ ID NO: 215) |
| R10P1A7 | IGF2BP3-001 |
| | (KIQEILTQV) |
| | (SEQ ID NO: 123) |
| R4P1D10, R4P3F9, R4P3H3 | COL6A3-002 (FLLDGSANV) |
| | (SEQ ID NO: 238) |
| R36P3F9, R52P2G11, R53P2A9 | DCAF4L2-001 |
| | (ILQDGQFLV) |
| | (SEQ ID NO: 193) |
| R26P1A9, R26P2A6, R26P3H1, | MAGEA1-003 (KVLEYVIKV) |
| R35P3A4, R37P1C9, R37P1H1, | (SEQ ID NO: 202) |
| R42P3A9, R43P3F2, R43P3G5, | |
| R59P2E7 | |
| R11KEA, R11P3D3, R16P1C10, | PRAME-004 (SLLQHLIGL) |
| R16P1E8, R17P1A9, R17P1D7, | (SEQ ID NO: 147) |
| R17P1G3, R17P2B6, R11P3D3KE | |
| R39P1C12, R39P1F5, R40P1C2, | SPINK2-001 (ALSVLRLAL) |
| R41P3E6, R43P3G4, R44P3B3, | (SEQ ID NO: 248) |
| R44P3E7, R49P2B7, R55P1G7, | |
| R59P2A7 | |

In an aspect, tumor associated antigen (TAA) peptides that are capable of use with the methods and embodiments described herein include, for example, those listed in Table 3 and those TAA peptides described in U.S. Publication 20160187351, U.S. Publication 20170165335, U.S. Publication 20170035807, U.S. Publication 20160280759, U.S. Publication 20160287687, U.S. Publication 20160346371, U.S. Publication 20160368965, U.S. Publication 20170022251, U.S. Publication 20170002055, U.S. Publication 20170029486, U.S. Publication 20170037089, U.S. Publication 20170136108, U.S. Publication 20170101473, U.S. Publication 20170096461, U.S. Publication 20170165337, U.S. Publication 20170189505, U.S. Publication 20170173132, U.S. Publication 20170296640, U.S. Publication 20170253633, U.S. Publication 20170260249, U.S. Publication 20180051080, U.S. Publication No. 20180164315, U.S. Publication 20180291082, U.S. Publication 20180291083, U.S. Publication 20190255110, U.S. Pat. Nos. 9,717,774, 9,895,415, U.S. Publication 20190247433, U.S. Publication 20190292520, U.S. Publication 20200085930, U.S. Pat. Nos. 10,336,809, 10,131,703, 10,081,664, 10,081,664, 10,093,715, 10,583,573, and US20200085930, the contents of each of these publications, sequences, and sequence listings described therein are herein incorporated by reference in their entireties.

In another aspect, the dimer Z1Z2 may be T cell dimeric signaling modules, such as CD3δ/ε, CD3γ/ε, and CD247 ζ/ζ or ζ/η, a dimer of a TCRα variable region (Vα) and a TCRβ variable region (Vβ), a dimer of immunoglobulin heavy chain variable region (VH) and immunoglobulin light chain variable region (VL), a dimer of Vα and VH, a dimer of Vα and VL, a dimer of Vβ and VH, or a dimer of Vβ and VL.

In another aspect, Y1Y2 may be CD8α chain and CD8β chain or any other suitable dimeric membrane receptors, preferably those expressed in the CD8+ T cells and/or in the CD4+ T cells.

Furin is a ubiquitous subtilisin-like proprotein convertase, whose natural substrates include certain serum proteins and growth factor receptors, such as the insulin-like growth factor receptor. The consensus sequence for furin cleavage is RXXR (SEQ ID NO: 7) but the potential for actual cleavage is dependent on substrate tertiary structure and the amino acids immediately surrounding the recognition site. Addition of a furin cleavage site plus the linker sequences (GSG or SGSG (SEQ ID NO: 8)) may enable highly efficient gene expression.

In one aspect, a nucleotide sequence of furin-linker-2A peptide arranged in tandem may be positioned between Z1 and Z2, between Z1 and Y1, between Z1 and Y2, between Z2 and Y1, between Z2 and Y2, and/or between Y1 and Y2. The furin may have a consensus sequence of RXXR (SEQ ID NO: 7), e.g., RAKR (SEQ ID NO: 2). The linker sequence may be GSG or SGSG (SEQ ID NO: 8). The 2A peptide may be selected from P2A (SEQ ID NO: 3), T2A (SEQ ID NO: 4), E2A (SEQ ID NO: 5), F2A (SEQ ID NO: 6), or any combination thereof.

In another aspect, a nucleotide sequence of linker-2A peptide arranged in tandem may be positioned between Z1 and Z2, between Z1 and Y1, between Z1 and Y2, between Z2 and Y1, between Z2 and Y2, and/or between Y1 and Y2. The linker sequence may be GSG or SGSG (SEQ ID NO: 8). The 2A peptide may be selected from P2A (SEQ ID NO: 3), T2A (SEQ ID NO: 4), E2A (SEQ ID NO: 5), F2A (SEQ ID NO: 6), or any combination thereof.

In an aspect, engineered (or transduced) γδ T cells can be expanded ex vivo without stimulation by an antigen presenting cell or aminobisphosphonate. Antigen reactive engineered T cells of the present disclosure may be expanded ex vivo and in vivo. In another aspect, an active population of engineered γδ T cells of the present disclosure may be expanded ex vivo without antigen stimulation by an antigen presenting cell, an antigenic peptide, a non-peptide molecule, or a small molecule compound, such as an aminobisphosphonate but using certain antibodies, cytokines, mitogens, or fusion proteins, such as IL-17 Fc fusion, MICA Fc fusion, and CD70 Fc fusion. Examples of antibodies that can be used in the expansion of a γδ T-cell population include anti-CD3, anti-CD27, anti-CD30, anti-CD70, anti-OX40, anti-NKG2D, or anti-CD2 antibodies, examples of cytokines may include IL-2, IL-15, IL-12, IL-21, IL-18, IL-9, IL-7, and/or IL-33, and examples of mitogens may include CD70 the ligand for human CD27, phytohaemagglutinin (PHA), concavalin A (ConA), pokeweed mitogen (PWM), protein peanut agglutinin (PNA), soybean agglutinin (SBA), Les culinaris agglutinin (LCA), Pisum sativum agglutinin (PSA), Helix pomatia agglutinin (HPA), Vicia graminea Lectin (VGA) or another suitable mitogen capable of stimulating T-cell proliferation. In another aspect, a population of engineered γδ T cells can be expanded in less than 60 days, less than 48 days, less than 36 days, less than 24 days, less than 12 days, or less than 6 days. In another aspect, a population of engineered γδ T cells can be expanded from about 7 days to about 49 days, about 7 days to about 42 days, from about 7 days to about 35 days, from about 7 days to about 28 days, from about 7 days to about 21 days, or from about 7 days to about 14 days.

In another aspect, the present disclosure provides methods for the ex vivo expansion of a population of engineered γδ T-cells for adoptive transfer therapy. Engineered γδ T cells of the disclosure may be expanded ex vivo. Engineered γδ T cells of the disclosure can be expanded in vitro without activation by APCs, or without co-culture with APCs, and aminophosphates.

### Methods of Treatment

Compositions containing engineered γδ T cells described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. An engineered γδ T-cell can also be administered to lessen a likelihood of developing, contracting, or worsening a condition. Effective amounts of a population of engineered γδ T-cells for therapeutic use can vary based on the severity and course of the disease or condition, previous therapy, the subject's health status, weight, and/or response to the drugs, and/or the judgment of the treating physician.

The composition of the present disclosure may also include one or more adjuvants. Adjuvants are substances that non-specifically enhance or potentiate the immune response (e.g., immune responses mediated by CD8-positive T cells and helper-T (TH) cells to an antigen and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel, 1995). Also cytokines may be used. Several cytokines have been directly linked to influencing dendritic cell migration to lymphoid tissues (e.g., TNF−), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12, IL-15, IL-23, IL-7, IFN-alpha. IFN-beta) (Gabrilovich et al., 1996).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Preferred adjuvants are anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), virosomes, and/or interleukin (IL)-1, IL-2, IL-4, IL-7, IL-12, IL-13, IL-15, IL-21, and IL-23.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the invention, the adjuvant is cyclophosphamide, imiquimod or resiquimod. Even more preferred adjuvants are Montanide IMS 1312, Montanide ISA 206, Montanide ISA 50V, Montanide ISA-51, poly-ICLC (Hiltonol®) and anti-CD40 mAB, or combinations thereof.

Engineered γδ T cells of the present disclosure can be used to treat a subject in need of treatment for a condition, for example, a cancer described herein.

A method of treating a condition (e.g., ailment) in a subject with γδ T cells may include administering to the subject a therapeutically effective amount of engineered γδ T cells. γδ T cells of the present disclosure may be administered at various regimens (e.g., timing, concentration, dosage, spacing between treatment, and/or formulation). A subject can also be preconditioned with, for example, chemotherapy, radiation, or a combination of both, prior to receiving engineered γδ T cells of the present disclosure. A population of engineered γδ T cells may also be frozen or cryopreserved prior to being administered to a subject. A population of engineered γδ T cells can include two or more cells that express identical, different, or a combination of identical and different tumor recognition moieties. For instance, a population of engineered γδ T-cells can include several distinct engineered γδ T cells that are designed to recognize different antigens, or different epitopes of the same antigen.

γδ T cells of the present disclosure may be used to treat various conditions. In an aspect, engineered γδ T cells of the present disclosure may be used to treat a cancer, including solid tumors and hematologic malignancies. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor.

In an aspect, engineered γδ T cells of the present disclosure may be used to treat an infectious disease. In another aspect, engineered γδ T cells of the present disclosure may be used to treat an infectious disease, an infectious disease may be caused a virus. In yet another aspect, engineered γδ T cells of the present disclosure may be used to treat an immune disease, such as an autoimmune disease.

Treatment with γδ T cells of the present disclosure may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered γδ T cells of the present disclosure.

In another aspect, administration of engineered γδ T cells of the present disclosure to a subject may modulate the activity of endogenous lymphocytes in a subject's body. In another aspect, administration of engineered γδ T cells to a subject may provide an antigen to an endogenous T-cell and may boost an immune response. In another aspect, the memory T cell may be a CD4+ T-cell. In another aspect, the memory T cell may be a CD8+ T-cell. In another aspect, administration of engineered γδ T cells of the present disclosure to a subject may activate the cytotoxicity of another immune cell. In another aspect, the other immune cell may be a CD8+ T-cell. In another aspect, the other immune cell may be a Natural Killer T-cell. In another aspect, administration of engineered γδ T-cells of the present disclosure to a subject may suppress a regulatory T-cell. In another aspect, the regulatory T-cell may be a FOX3+ Treg cell. In another aspect, the regulatory T-cell may be a FOX3-Treg cell. Non-limiting examples of cells whose activity can be modulated by engineered γδ T cells of the disclosure may include: hematopioietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells.

During most bone marrow transplants, a combination of cyclophosphamide with total body irradiation may be conventionally employed to prevent rejection of the hematopietic stem cells (HSC) in the transplant by the subject's immune system. In an aspect, incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of γδ T-cells into humans may require the co-administration of γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. In another aspect, the disclosure provides a method for administrating engineered γδ T cells to a subject without the co-administration of a native cytokine or modified versions thereof, such as IL-2, IL-15, IL-12, IL-21. In another aspect, engineered γδ T cells can be administered to a subject without co-administration with IL-2. In another aspect, engineered γδ T cells may be administered to a subject during a procedure, such as a bone marrow transplant without the co-administration of IL-2.

### Methods of Administration

One or multiple engineered γδ T cell populations may be administered to a subject in any order or simultaneously. If simultaneously, the multiple engineered γδ T cell can be provided in a single, unified form, such as an intravenous injection, or in multiple forms, for example, as multiple intravenous infusions, s.c, injections or pills. Engineered γδ T-cells can be packed together or separately, in a single package or in a plurality of packages. One or all of the engineered γδ T cells can be given in multiple doses. If not simultaneous, the timing between the multiple doses may vary to as much as about a week, a month, two months, three months, four months, five months, six months, or about a year. In another aspect, engineered γδ T cells can expand within a subject's body, in vivo, after administration to a subject. Engineered γδ T cells can be frozen to provide cells for multiple treatments with the same cell preparation. Engineered γδ T cells of the present disclosure, and pharmaceutical compositions comprising the same, can be packaged as a kit. A kit may include instructions (e.g., written instructions) on the use of engineered γδ T cells and compositions comprising the same.

In another aspect, a method of treating a cancer comprises administering to a subject a therapeutically-effective amount of engineered γδ T cells, in which the administration treats the cancer. In another embodiments, the therapeutically-effective amount of engineered γδ T cells may be administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. In another aspect, the therapeutically-effective amount of the engineered γδ T cells may be administered for at least one week. In another aspect, the therapeutically-effective amount of engineered γδ T cells may be administered for at least two weeks.

Engineered γδ T-cells described herein can be administered before, during, or after the occurrence of a disease or condition, and the timing of administering a pharmaceutical composition containing an engineered γδ T-cell can vary. For example, engineered γδ T cells can be used as a prophylactic and can be administered continuously to subjects with a propensity to conditions or diseases in order to lessen the likelihood of occurrence of the disease or condition. Engineered γδ T-cells can be administered to a subject during or as soon as possible after the onset of the symptoms. The administration of engineered γδ T cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In another aspect, the administration of engineered γδ T cells of the present disclosure may be an intravenous administration. One or multiple dosages of engineered γδ T cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered γδ T cells can be administered years after onset of the cancer and before or after other treatments. In another aspect, engineered γδ T cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

### Preservation

In an aspect, γδ T cells may be formulated in freezing media and placed in cryogenic storage units such as liquid nitrogen freezers (−196° C.) or ultra-low temperature freezers (−65° C., −80° C., −120° C., or −150° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved γδ T cells can be thawed and further processed by stimulation with antibodies, proteins, peptides, and/or cytokines as described herein. The cryopreserved γδ T-cells can be thawed and genetically modified with viral vectors (including retroviral, adeno-associated virus (AAV), and lentiviral vectors) or non-viral means (including RNA, DNA, e.g., transposons, and proteins) as described herein. The modified γδ T cells can be further cryopreserved to generate cell banks in quantities of at least about 1, 5, 10, 100, 150, 200, 500 vials at about at least 101, 102, 103, 104, 105, 106, 107, 108, 109, or at least about 1010 cells per mL in freeze media. The cryopreserved cell banks may retain their functionality and can be thawed and further stimulated and expanded. In another aspect, thawed cells can be stimulated and expanded in suitable closed vessels, such as cell culture bags and/or bioreactors, to generate quantities of cells as allogeneic cell product. Cryopreserved γδ T cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In another aspect, no preservatives may be used in the formulation. Cryopreserved γδ T-cells can be thawed and infused into multiple patients as allogeneic off-the-shelf cell product.

In an aspect, engineered γδ T-cell described herein may be present in a composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

In an aspect, methods described herein may be used to produce autologous or allogenic products according to an aspect of the disclosure.

In an aspect, vectors, constructs, or sequences described herein may comprise about 80%, about 85%, about 90%, about 85%, about 96%, about 97%, about 98%, or about 99% to any of SEQ ID NO: 1-97 and 265-266. A sequence “at least 85% identical to a reference sequence” is a sequence having, on its entire length, 85%, or more, in particular 90%, 91%, 92%, 93%, 94%, 95%, 96%, 97%, 98% or 99% sequence identity with the entire length of the reference sequence.

In the context of the present application, the “percentage of identity” is calculated using a global pairwise alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino acid substitutions” may be conservative or non-conservative. Preferably, substitutions are conservative substitutions, in which one amino acid is substituted for another amino acid with similar structural and/or chemical properties.

In an embodiment, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine(S), threonine (T); Group 2: cysteine (C), serine(S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A→I, T→V, A→M, T→I, A→V, T→G, and/or T→S.

In a further embodiment, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

In another embodiment, conservative substitutions may be made in accordance with Table 1. Methods for predicting tolerance to protein modification may be found in, for example, Guo et al., Proc. Natl. Acad. Sci., USA, 101 (25): 9205-9210 (2004), the contents of which are incorporated by reference in their entirety.

TABLE A
Conservative Amino Acid substitution
Conservative Amino Acid Substitutions

| | Substitutions |
|---|---|
| Amino Acid | (others are known in the art) |
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

In an aspect, sequences described herein may include 1, 2, 3, 4, 5, 10, 15, 20, 25, or 30 amino acid or nucleotide mutations, substitutions, deletions. In an aspect, any one of SEQ ID NO: 1-97 and 265-266 may include 1, 2, 3, 4, 5, 10, 15, 20, 25, or 30 mutations, substitutions, or deletions. In yet another aspect, the mutations or substitutions are conservative amino acid substitutions.

In another embodiment, conservative substitutions may be those shown in Table B under the heading of “conservative substitutions.” If such substitutions result in a change in biological activity, then more substantial changes, denominated “exemplary substitutions” in Table B, may be introduced and the products screened if needed.

TABLE B
Amino Acid substitution
Amino Acid Substitutions

| Original Residue | | |
|---|---|---|
| (naturally occurring | Conservative | Exemplary |
| amino acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

### Example 1

TABLE 2
DNA and protein sequences

| SEQ | | |
|---|---|---|
| ID NO: | Description | Sequence |
| 1 | MSCV | Tgaaagaccccacctgtaggtttggcaagctagcttaagtaacgccattttgcaaggc |
| | promoter | atggaaaatacataactgagaatagagaagttcagatcaaggttaggaacagagag |
| | | acagcagaatatgggccaaacaggatatctgtggtaagcagttcctgccccggctca |
| | | gggccaagaacagatggtccccagatgcggtcccgccctcagcagtttctagagaac |
| | | catcagatgtttccagggtgccccaaggacctgaaaatgaccctgtgccttatttgaact |
| | | aaccaatcagttcgcttctcgcttctgttcgcgcgcttctgctccccgagctcaataaaag |
| | | agcccacaacccctcact |
| 2 | Furin | RAKR |
| 3 | P2A | ATNFSLLKQAGDVEENPGP |
| 4 | T2A | EGRGSLLTCGDVEENPGP |
| 5 | E2A | QCTNYALLKLAGDVESNPGP |
| 6 | F2A | VKQTLNFDLLKLAGDVESNPGP |
| 7 | Furin | RXXR |
| | consensus | |
| 8 | Linker | SGSG |
| 9 | WPRE | cagtctgacgtacgcgtaatcaacctctggattacaaaatttgtgaaagattgactggtat |
| | | tcttaactatgttgctccttttacgctatgtggatacgctgctttaatgcctttgtatcatgctatt |
| | | gcttcccgtatggctttcattttctcctccttgtataaatcctggttgctgtctctttatgaggagt |
| | | tgtggcccgttgtcaggcaacgtggcgtggtgtgcactgtgtttgctgacgcaaccccca |
| | | ctggttggggcattgccaccacctgtcagctcctttccgggactttcgctttccccctcccta |
| | | ttgccacggcggaactcatcgccgcctgccttgcccgctgctggacaggggctcggct |
| | | gttgggcactgacaattccgtggtgttgtcggggaagctgacgtcctttccatggctgctc |
| | | gcctgtgttgccacctggattctgcgcgggacgtccttctgctacgtcccttcggccctca |
| | | atccagcggaccttccttcccgcggcctgctgccggctctgcggcctcttccgcgtcttcg |
| | | ccttcgccctcagacgagtcggatctccctttgggccgcctccccgcc |
| 10 | X protein | Ggggaagctgacgtcctttcc |
| | promoter | |
| 11 | CD8 alpha | MALPVTALLLPLALLLHAARPSQFRVSPLDRTWNLGETVELKC |
| | chain | QVLLSNPTSGCSWLFQPRGAAASPTFLLYLSQNKPKAAEGLD |
| | | TQRFSGKRLGDTFVLTLSDFRRENEGYYFCSALSNSIMYFSH |
| | | FVPVFLPAKPTTTPAPRPPTPAPTIASQPLSLRPEACRPAAGG |
| | | AVHTRGLDFACDIYIWAPLAGTCGVLLLSLVITLYCNHRNRRR |
| | | VCKCPRPVVKSGDKPSLSARYV |
| 12 | CD8 beta | MRPRLWLLLAAQLTVLHGNSVLQQTPAYIKVQTNKMVMLSCE |
| | chain | AKISLSNMRIYWLRQRQAPSSDSHHEFLALWDSAKGTIHGEE |
| | | VEQEKIAVFRDASRFILNLTSVKPEDSGIYFCMIVGSPELTFGK |
| | | GTQLSVVDFLPTTAQPTKKSTLKKRVCRLPRPETQKGPLCSPI |
| | | TLGLLVAGVLVLLVSLGVAIHLCCRRRRARLRFMKQPQGEGIS |
| | | GTFVPQCLHGYYSNTTTSQKLLNPWILKT |
| 13 | R11KEA alpha | MEKNPLAAPLLILWFHLDCVSSILNVEQSPQSLHVQEGDSTNF |
| | chain | TCSFPSSNFYALHWYRKETAKSPEALFVMTLNGDEKKKGRIS |
| | | ATLNTKEGYSYLYIKGSQPEDSATYLCALYNNNDMRFGAGTR |
| | | LTVKPNIQNPDPAVYQLRDSKSSDKSVCLFTDFDSQTNVSQS |
| | | KDSDVYITDKTVLDMRSMDFKSNSAVAWSNKSDFACANAFN |
| | | NSIIPEDTFFPSPESSCDVKLVEKSFETDTNLNFQNLSVIGFRIL |
| | | LLKVAGFNLLMTLRLWSS |
| 14 | R11KE beta | MDSWTFCCVSLCILVAKHTDAGVIQSPRHEVTEMGQEVTLRC |
| | chain | KPISGHNSLFWYRETMMRGLELLIYFNNNVPIDDSGMPEDRF |
| | | SAKMPNASFSTLKIQPSEPRDSAVYFCASSPGSTDTQYFGPG |
| | | TRLTVLEDLKNVFPPEVAVFEPSEAEISHTQKATLVCLATGFY |
| | | PDHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 15 | R20P1H7 | MEKMLECAFIVLWLQLGWLSGEDQVTQSPEALRLQEGESSS |
| | alpha chain | LNCSYTVSGLRGLFWYRQDPGKGPEFLFTLYSAGEEKEKERL |
| | | KATLTKKESFLHITAPKPEDSATYLCAVQGENSGYSTLTFGKG |
| | | TMLLVSPDIQNPDPAVYQLRDSKSSDKSVCLFTDFDSQTNVS |
| | | QSKDSDVYITDKTVLDMRSMDFKSNSAVAWSNKSDFACANA |
| | | FNNSIIPEDTFFPSPESSCDVKLVEKSFETDTNLNFQNLSVIGF |
| | | RILLLKVAGFNLLMTLRLWSS |
| 16 | R20P1H7 beta | MGPQLLGYVVLCLLGAGPLEAQVTQNPRYLITVTGKKLTVTC |
| | chain | SQNMNHEYMSWYRQDPGLGLRQIYYSMNVEVTDKGDVPEG |
| | | YKVSRKEKRNFPLILESPSPNQTSLYFCASSLGPGLAAYNEQF |
| | | FGPGTRLTVLEDLKNVFPPEVAVFEPSEAEISHTQKATLVCLA |
| | | TGFYPDHVELSWWVNGKEVHSGVSTDPQPLKEQPALNDSR |
| | | YCLSSRLRVSATFWQNPRNHFRCQVQFYGLSENDEWTQDR |
| | | AKPVTQIVSAEAWGRADCGFTSESYQQGVLSATILYEILLGKA |
| | | TLYAVLVSALVLMAMVKRKDSRG |
| 17 | R7P1D5 alpha | MKTFAGFSFLFLWLQLDCMSRGEDVEQSLFLSVREGDSSVIN |
| | chain | CTYTDSSSTYLYWYKQEPGAGLQLLTYIFSNMDMKQDQRLTV |
| | | LLNKKDKHLSLRIADTQTGDSAIYFCAEYSSASKIIFGSGTRLSI |
| | | RPNIQNPDPAVYQLRDSKSSDKSVCLFTDFDSQTNVSQSKDS |
| | | DVYITDKTVLDMRSMDFKSNSAVAWSNKSDFACANAFNNSII |
| | | PEDTFFPSPESSCDVKLVEKSFETDTNLNFQNLSVIGFRILLLK |
| | | VAGFNLLMTLRLWSS |
| 18 | R7P1D5 beta | MGSWTLCCVSLCILVAKHTDAGVIQSPRHEVTEMGQEVTLRC |
| | chain | KPISGHDYLFWYRQTMMRGLELLIYFNNNVPIDDSGMPEDRF |
| | | SAKMPNASFSTLKIQPSEPRDSAVYFCASRANTGELFFGEGS |
| | | RLTVLEDLKNVFPPEVAVFEPSEAEISHTQKATLVCLATGFYP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 19 | R10P2G12 | MLTASLLRAVIASICVVSSMAQKVTQAQTEISVVEKEDVTLDC |
| | alpha chain | VYETRDTTYYLFWYKQPPSGELVFLIRRNSFDEQNEISGRYS |
| | | WNFQKSTSSFNFTITASQVVDSAVYFCALSEGNSGNTPLVFG |
| | | KGTRLSVIANIQNPDPAVYQLRDSKSSDKSVCLFTDFDSQTNV |
| | | SQSKDSDVYITDKTVLDMRSMDFKSNSAVAWSNKSDFACAN |
| | | AFNNSIIPEDTFFPSPESSCDVKLVEKSFETDTNLNFQNLSVIG |
| | | FRILLLKVAGFNLLMTLRLWSS |
| 20 | R10P2G12 | MGIRLLCRVAFCFLAVGLVDVKVTQSSRYLVKRTGEKVFLEC |
| | beta chain | VQDMDHENMFWYRQDPGLGLRLIYFSYDVKMKEKGDIPEGY |
| | | SVSREKKERFSLILESASTNQTSMYLCASSLSSGSHQETQYF |
| | | GPGTRLLVLEDLKNVFPPEVAVFEPSEAEISHTQKATLVCLAT |
| | | GFYPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSESYQQGVLSATILYEILLGKAT |
| | | LYAVLVSALVLMAMVKRKDSRG |
| 21 | R10P1A7 | MKTFAGFSFLFLWLQLDCMSRGEDVEQSLFLSVREGDSSVIN |
| | alpha chain | CTYTDSSSTYLYWYKQEPGAGLQLLTYIFSNMDMKQDQRLTV |
| | | LLNKKDKHLSLRIADTQTGDSAIYFCAESKETRLMFGDGTQLV |
| | | VKPNIQNPDPAVYQLRDSKSSDKSVCLFTDFDSQTNVSQSKD |
| | | SDVYITDKTVLDMRSMDFKSNSAVAWSNKSDFACANAFNNSI |
| | | IPEDTFFPSPESSCDVKLVEKSFETDTNLNFQNLSVIGFRILLL |
| | | KVAGFNLLMTLRLWSS |
| 22 | R10P1A7 beta | MLLLLLLLGPGISLLLPGSLAGSGLGAWSQHPSVWICKSGTSV |
| | chain | KIECRSLDFQATTMFWYRQFPKQSLMLMATSNEGSKATYEQ |
| | | GVEKDKFLINHASLTLSTLTVTSAHPEDSSFYICSARAGGHEQ |
| | | FFGPGTRLTVLEDLKNVFPPEVAVFEPSEAEISHTQKATLVCL |
| | | ATGFYPDHVELSWVWNGKEVHSGVSTDPQPLKEQPALNDS |
| | | RYCLSSRLRVSATFWQNPRNHFRCQVQFYGLSENDEWTQD |
| | | RAKPVTQIVSAEAWGRADCGFTSESYQQGVLSATILYEILLGK |
| | | ATLYAVLVSALVLMAMVKRKDSRG |
| 23 | R4P1D10 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAVNFHDKIIFGKGTRL |
| | | HILPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 24 | R4P1D10 beta | MGFRLLCCVAFCLLGAGPVDSGVTQTPKHLITATGQRVTLRC |
| | chain | SPRSGDLSVYWYQQSLDQGLQFLIHYYNGEERAKGNILERFS |
| | | AQQFPDLHSELNLSSLELGDSALYFCASSVASAYGYTFGSGT |
| | | RLTVVEDLNKVFPPEVAVFEPSEAEISHTQKATLVCLATGFFP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSVSYQQGVLSATILYEILLGKATLYAV |
| | | LVSALVLMAMVKRKDF |
| 25 | R4P3F9 alpha | MKSLRVLLVILWLQLSWVWSQQKEVEQNSGPLSVPEGAIASL |
| | chain | NCTYSDRGSQSFFWYRQYSGKSPELIMFIYSNGDKEDGRFT |
| | | AQLNKASQYVSLLIRDSQPSDSATYLCAAYSGAGSYQLTFGK |
| | | GTKLSVIPNIQNPDPAVYQLRDSKSSDKSVCLFTDFDSQTNVS |
| | | QSKDSDVYITDKTVLDMRSMDFKSNSAVAWSNKSDFACANA |
| | | FNNSIIPEDTFFPSPESSCDVKLVEKSFETDTNLNFQNLSVIGF |
| | | RILLLKVAGFNLLMTLRLWSS |
| 26 | R4P3F9 beta | MGFRLLCCVAFCLLGAGPVDSGVTQTPKHLITATGQRVTLRC |
| | chain | SPRSGDLSVYWYQQSLDQGLQFLIQYYNGEERAKGNILERFS |
| | | AQQFPDLHSELNLSSLELGDSALYFCASSVESSYGYTFGSGT |
| | | RLTVVEDLNKVFPPEVAVFEPSEAEISHTQKATLVCLATGFFP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSVSYQQGVLSATILYEILLGKATLYAV |
| | | LVSALVLMAMVKRKDF |
| 27 | R4P3H3 alpha | MKSLRVLLVILWLQLSWVWSQQKEVEQNSGPLSVPEGAIASL |
| | chain | NCTYSDRGSQSFFWYRQYSGKSPELIMFIYSNGDKEDGRFT |
| | | AQLNKASQYVSLLIRDSQPSDSATYLCAVKAGNQFYFGTGTS |
| | | LTVIPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 28 | R4P3H3 beta | MGTRLLCWVVLGFLGTDHTGAGVSQSPRYKVAKRGQDVALR |
| | chain | CDPISGHVSLFWYQQALGQGPEFLTYFQNEAQLDKSGLPSD |
| | | RFFAERPEGSVSTLKIQRTQQEDSAVYLCASSLLTSGGDNEQ |
| | | FFGPGTRLTVLEDLKNVFPPEVAVFEPSEAEISHTQKATLVCL |
| | | ATGFYPDHVELSWWVNGKEVHSGVSTDPQPLKEQPALNDS |
| | | RYCLSSRLRVSATFWQNPRNHFRCQVQFYGLSENDEWTQD |
| | | RAKPVTQIVSAEAWGRADCGFTSESYQQGVLSATILYEILLGK |
| | | ATLYAVLVSALVLMAMVKRKDSRG |
| 29 | R36P3F9 | METLLGVSLVILWLQLARVNSQQGEEDPQALSIQEGENATMN |
| | alpha chain | CSYKTSINNLQWYRQNSGRGLVHLILIRSNEREKHSGRLRVTL |
| | | DTSKKSSSLLITASRAADTASYFCATVSNYQLIWGAGTKLIIKP |
| | | DIQNPDPAVYQLRDSKSSDKSVCLFTDFDSQTNVSQSKDSDV |
| | | YITDKTVLDMRSMDFKSNSAVAWSNKSDFACANAFNNSIIPE |
| | | DTFFPSPESSCDVKLVEKSFETDTNLNFQNLSVIGFRILLLKVA |
| | | GFNLLMTLRLWSS |
| 30 | R36P3F9 beta | MGPQLLGYVVLCLLGAGPLEAQVTQNPRYLITVTGKKLTVTC |
| | chain | SQNMNHEYMSWYRQDPGLGLRQIYYSMNVEVTDKGDVPEG |
| | | YKVSRKEKRNFPLILESPSPNQTSLYFCASSSTSGGLSGETQ |
| | | YFGPGTRLLVLEDLKNVFPPEVAVFEPSEAEISHTQKATLVCL |
| | | ATGFYPDHVELSWWVNGKEVHSGVSTDPQPLKEQPALNDS |
| | | RYCLSSRLRVSATFWQNPRNHFRCQVQFYGLSENDEWTQD |
| | | RAKPVTQIVSAEAWGRADCGFTSESYQQGVLSATILYEILLGK |
| | | ATLYAVLVSALVLMAMVKRKDSRG |
| 31 | R52P2G11 | MKKHLTTFLVILWLYFYRGNGKNQVEQSPQSLIILEGKNCTLQ |
| | alpha chain | CNYTVSPFSNLRWYKQDTGRGPVSLTIMTFSENTKSNGRYTA |
| | | TLDADTKQSSLHITASQLSDSASYICVVSAYGKLQFGAGTQVV |
| | | VTPDIQNPDPAVYQLRDSKSSDKSVCLFTDFDSQTNVSQSKD |
| | | SDVYITDKTVLDMRSMDFKSNSAVAWSNKSDFACANAFNNSI |
| | | IPEDTFFPSPESSCDVKLVEKSFETDTNLNFQNLSVIGFRILLL |
| | | KVAGFNLLMTLRLWSS |
| 32 | R52P2G11 | MDSWTFCCVSLCILVAKHTDAGVIQSPRHEVTEMGQEVTLRC |
| | beta chain | KPISGHNSLFWYRQTMMRGLELLIYFNNNVPIDDSGMPEDRF |
| | | SAKMPNASFSTLKIQPSEPRDSAVYFCASSLGSPDGNQPQHF |
| | | GDGTRLSILEDLNKVFPPEVAVFEPSEAEISHTQKATLVCLAT |
| | | GFFPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSVSYQQGVLSATILYEILLGKAT |
| | | LYAVLVSALVLMAMVKRKDF |
| 33 | R53P2A9 | MACPGFLWALVISTCLEFSMAQTVTQSQPEMSVQEAETVTLS |
| | alpha chain | CTYDTSESDYYLFWYKQPPSRQMILVIRQEAYKQQNATENRF |
| | | SVNFQKAAKSFSLKISDSQLGDAAMYFCAYNSYAGGTSYGKL |
| | | TFGQGTILTVHPNIQNPDPAVYQLRDSKSSDKSVCLFTDFDS |
| | | QTNVSQSKDSDVYITDKTVLDMRSMDFKSNSAVAWSNKSDF |
| | | ACANAFNNSIIPEDTFFPSPESSCDVKLVEKSFETDTNLNFQN |
| | | LSVIGFRILLLKVAGFNLLMTLRLWSS |
| 34 | R53P2A9 beta | MGPGLLCWVLLCLLGAGPVDAGVTQSPTHLIKTRGQQVTLRC |
| | chain | SPISGHKSVSWYQQVLGQGPQFIFQYYEKEERGRGNFPDRF |
| | | SARQFPNYSSELNVNALLLGDSALYLCASSLDGTSEQYFGPG |
| | | TRLTVTEDLKNVFPPEVAVFEPSEAEISHTQKATLVCLATGFY |
| | | PDHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 35 | R26P1A9 | METLLGVSLVILWLQLARVNSQQGEEDPQALSIQEGENATMN |
| | alpha chain | CSYKTSINNLQWYRQNSGRGLVHLILIRSNEREKHSGRLRVTL |
| | | DTSKKSSSLLITASRAADTASYFCLIGASGSRLTFGEGTQLTV |
| | | NPDIQNPDPAVYQLRDSKSSDKSVCLFTDFDSQTNVSQSKDS |
| | | DVYITDKTVLDMRSMDFKSNSAVAWSNKSDFACANAFNNSII |
| | | PEDTFFPSPESSCDVKLVEKSFETDTNLNFQNLSVIGFRILLLK |
| | | VAGFNLLMTLRLWSS |
| 36 | R26P1A9 beta | MGSWTLCCVSLCILVAKHTDAGVIQSPRHEVTEMGQEVTLRC |
| | chain | KPISGHDYLFWYRQTMMRGLELLIYFNNNVPIDDSGMPEDRF |
| | | SAKMPNASFSTLKIQPSEPRDSAVYFCASSYFGWNEKLFFGS |
| | | GTQLSVLEDLNKVFPPEVAVFEPSEAEISHTQKATLVCLATGF |
| | | FPDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSVSYQQGVLSATILYEILLGKATLYA |
| | | VLVSALVLMAMVKRKDF |
| 37 | R26P2A6 | MMKSLRVLLVILWLQLSWVWSQQKEVEQDPGPLSVPEGAIV |
| | alpha chain | SLNCTYSNSAFQYFMWYRQYSRKGPELLMYTYSSGNKEDG |
| | | RFTAQVDKSSKYISLFIRDSQPSDSATYLCAMSDVSGGYNKLI |
| | | FGAGTRLAVHPYIQNPDPAVYQLRDSKSSDKSVCLFTDFDSQ |
| | | TNVSQSKDSDVYITDKTVLDMRSMDFKSNSAVAWSNKSDFA |
| | | CANAFNNSIIPEDTFFPSPESSCDVKLVEKSFETDTNLNFQNL |
| | | SVIGFRILLLKVAGFNLLMTLRLWSS |
| 38 | R26P2A6 beta | MGPQLLGYVVLCLLGAGPLEAQVTQNPRYLITVTGKKLTVTC |
| | chain | SQNMNHEYMSWYRQDPGLGLRQIYYSMNVEVTDKGDVPEG |
| | | YKVSRKEKRNFPLILESPSPNQTSLYFCASTTPDGTDEQFFGP |
| | | GTRLTVLEDLKNVFPPEVAVFEPSEAEISHTQKATLVCLATGF |
| | | YPDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSESYQQGVLSATILYEILLGKATLYA |
| | | VLVSALVLMAMVKRKDSRG |
| 39 | R26P3H1 | MASAPISMLAMLFTLSGLRAQSVAQPEDQVNVAEGNPLTVKC |
| | alpha chain | TYSVSGNPYLFWYVQYPNRGLQFLLKYITGDNLVKGSYGFEA |
| | | EFNKSQTSFHLKKPSALVSDSALYFCAVRDMNRDDKIIFGKGT |
| | | RLHILPNIQNPDPAVYQLRDSKSSDKSVCLFTDFDSQTNVSQS |
| | | KDSDVYITDKTVLDMRSMDFKSNSAVAWSNKSDFACANAFN |
| | | NSIIPEDTFFPSPESSCDVKLVEKSFETDTNLNFQNLSVIGFRIL |
| | | LLKVAGFNLLMTLRLWSS |
| 40 | R26P3H1 beta | MSNQVLCCVVLCFLGANTVDGGITQSPKYLFRKEGQNVTLSC |
| | chain | EQNLNHDAMYWYRQDPGQGLRLIYYSQIVNDFQKGDIAEGY |
| | | SVSREKKESFPLTVTSAQKNPTAFYLCASSRAEGGEQYFGPG |
| | | TRLTVTEDLKNVFPPEVAVFEPSEAEISHTQKATLVCLATGFY |
| | | PDHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 41 | R35P3A4 | MTSIRAVFIFLWLQLDLVNGENVEQHPSTLSVQEGDSAVIKCT |
| | alpha chain | YSDSASNYFPWYKQELGKRPQUIDIRSNVGEKKDQRIAVTLN |
| | | KTAKHFSLHITETQPEDSAVYFCAASPTGGYNKLIFGAGTRLA |
| | | VHPYIQNPDPAVYQLRDSKSSDKSVCLFTDFDSQTNVSQSKD |
| | | SDVYITDKTVLDMRSMDFKSNSAVAWSNKSDFACANAFNNSI |
| | | IPEDTFFPSPESSCDVKLVEKSFETDTNLNFQNLSVIGFRILLL |
| | | KVAGFNLLMTLRLWSS |
| 42 | R35P3A4 beta | MSIGLLCCAALSLLWAGPVNAGVTQTPKFQVLKTGQSMTLQC |
| | chain | AQDMNHEYMSWYRQDPGMGLRLIHYSVGAGITDQGEVPNG |
| | | YNVSRSTTEDFPLRLLSAAPSQTSVYFCASSLGGASQEQYFG |
| | | PGTRLTVTEDLKNVFPPEVAVFEPSEAEISHTQKATLVCLATG |
| | | FYPDHVELSWWVNGKEVHSGVSTDPQPLKEQPALNDSRYCL |
| | | SSRLRVSATFWQNPRNHFRCQVQFYGLSENDEWTQDRAKP |
| | | VTQIVSAEAWGRADCGFTSESYQQGVLSATILYEILLGKATLY |
| | | AVLVSALVLMAMVKRKDSRG |
| 43 | R37P1C9 | MKLVTSITVLLSLGIMGDAKTTQPNSMESNEEEPVHLPCNHST |
| | alpha chain | ISGTDYIHWYRQLPSQGPEYVIHGLTSNVNNRMASLAIAEDRK |
| | | SSTLILHRATLRDAAVYYCILFNFNKFYFGSGTKLNVKPNIQNP |
| | | DPAVYQLRDSKSSDKSVCLFTDFDSQTNVSQSKDSDVYITDK |
| | | TVLDMRSMDFKSNSAVAWSNKSDFACANAFNNSIIPEDTFFP |
| | | SPESSCDVKLVEKSFETDTNLNFQNLSVIGFRILLLKVAGFNLL |
| | | MTLRLWSS |
| 44 | R37P1C9 beta | MGPGLLHWMALCLLGTGHGDAMVIQNPRYQVTQFGKPVTLS |
| | chain | CSQTLNHNVMYWYQQKSSQAPKLLFHYYDKDFNNEADTPDN |
| | | FQSRRPNTSFCFLDIRSPGLGDAAMYLCATSSGETNEKLFFG |
| | | SGTQLSVLEDLNKVFPPEVAVFEPSEAEISHTQKATLVCLATG |
| | | FFPDHVELSWWVNGKEVHSGVSTDPQPLKEQPALNDSRYCL |
| | | SSRLRVSATFWQNPRNHFRCQVQFYGLSENDEWTQDRAKP |
| | | VTQIVSAEAWGRADCGFTSVSYQQGVLSATILYEILLGKATLY |
| | | AVLVSALVLMAMVKRKDF |
| 45 | R37P1H1 | MTRVSLLWAVVVSTCLESGMAQTVTQSQPEMSVQEAETVTL |
| | alpha chain | SCTYDTSESNYYLFWYKQPPSRQMILVIRQEAYKQQNATENR |
| | | FSVNFQKAAKSFSLKISDSQLGDTAMYFCAFGYSGGGADGLT |
| | | FGKGTHLIIQPYIQNPDPAVYQLRDSKSSDKSVCLFTDFDSQT |
| | | NVSQSKDSDVYITDKTVLDMRSMDFKSNSAVAWSNKSDFAC |
| | | ANAFNNSIIPEDTFFPSPESSCDVKLVEKSFETDTNLNFQNLS |
| | | VIGFRILLLKVAGFNLLMTLRLWSS |
| 46 | R37P1H1 beta | MGPGLLCWALLCLLGAGLVDAGVTQSPTHLIKTRGQQVTLRC |
| | chain | SPKSGHDTVSWYQQALGQGPQFIFQYYEEEERQRGNFPDRF |
| | | SGHQFPNYSSELNVNALLLGDSALYLCASSNEGQGWEAEAF |
| | | FGQGTRLTVVEDLNKVFPPEVAVFEPSEAEISHTQKATLVCLA |
| | | TGFFPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSVSYQQGVLSATILYEILLGKAT |
| | | LYAVLVSALVLMAMVKRKDF |
| 47 | R42P3A9 | MKRILGALLGLLSAQVCCVRGIQVEQSPPDLILQEGANSTLRC |
| | alpha chain | NFSDSVNNLQWFHQNPWGQLINLFYIPSGTKQNGRLSATTVA |
| | | TERYSLLYISSSQTTDSGVYFCAVHNFNKFYFGSGTKLNVKP |
| | | NIQNPDPAVYQLRDSKSSDKSVCLFTDFDSQTNVSQSKDSDV |
| | | YITDKTVLDMRSMDFKSNSAVAWSNKSDFACANAFNNSIIPE |
| | | DTFFPSPESSCDVKLVEKSFETDTNLNFQNLSVIGFRILLLKVA |
| | | GFNLLMTLRLWSS |
| 48 | R42P3A9 beta | MLSPDLPDSAWNTRLLCHVMLCLLGAVSVAAGVIQSPRHLIK |
| | chain | EKRETATLKCYPIPRHDTVYWYQQGPGQDPQFLISFYEKMQS |
| | | DKGSIPDRFSAQQFSDYHSELNMSSLELGDSALYFCASSLLG |
| | | QGYNEQFFGPGTRLTVLEDLKNVFPPEVAVFEPSEAEISHTQ |
| | | KATLVCLATGFYPDHVELSWWVNGKEVHSGVSTDPQPLKEQ |
| | | PALNDSRYCLSSRLRVSATFWQNPRNHFRCQVQFYGLSEND |
| | | EWTQDRAKPVTQIVSAEAWGRADCGFTSESYQQGVLSATILY |
| | | EILLGKATLYAVLVSALVLMAMVKRKDSRG |
| 49 | R43P3F2 | MLTASLLRAVIASICVVSSMAQKVTQAQTEISVVEKEDVTLDC |
| | alpha chain | VYETRDTTYYLFWYKQPPSGELVFLIRRNSFDEQNEISGRYS |
| | | WNFQKSTSSFNFTITASQVVDSAVYFCALSNNNAGNMLTFGG |
| | | GTRLMVKPHIQNPDPAVYQLRDSKSSDKSVCLFTDFDSQTNV |
| | | SQSKDSDVYITDKTVLDMRSMDFKSNSAVAWSNKSDFACAN |
| | | AFNNSIIPEDTFFPSPESSCDVKLVEKSFETDTNLNFQNLSVIG |
| | | FRILLLKVAGFNLLMTLRLWSS |
| 50 | R43P3F2 beta | MLSPDLPDSAWNTRLLCHVMLCLLGAVSVAAGVIQSPRHLIK |
| | chain | EKRETATLKCYPIPRHDTVYWYQQGPGQDPQFLISFYEKMQS |
| | | DKGSIPDRFSAQQFSDYHSELNMSSLELGDSALYFCASSPTG |
| | | TSGYNEQFFGPGTRLTVLEDLKNVFPPEVAVFEPSEAEISHTQ |
| | | KATLVCLATGFYPDHVELSWWVNGKEVHSGVSTDPQPLKEQ |
| | | PALNDSRYCLSSRLRVSATFWQNPRNHFRCQVQFYGLSEND |
| | | EWTQDRAKPVTQIVSAEAWGRADCGFTSESYQQGVLSATILY |
| | | EILLGKATLYAVLVSALVLMAMVKRKDSRG |
| 51 | R43P3G5 | MEKNPLAAPLLILWFHLDCVSSILNVEQSPQSLHVQEGDSTNF |
| | alpha chain | TCSFPSSNFYALHWYRWETAKSPEALFVMTLNGDEKKKGRIS |
| | | ATLNTKEGYSYLYIKGSQPEDSATYLCALNRDDKIIFGKGTRL |
| | | HILPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 52 | R43P3G5 | MGIRLLCRVAFCFLAVGLVDVKVTQSSRYLVKRTGEKVFLEC |
| | beta chain | VQDMDHENMFWYRQDPGLGLRLIYFSYDVKMKEKGDIPEGY |
| | | SVSREKKERFSLILESASTNQTSMYLCASRLPSRTYEQYFGP |
| | | GTRLTVTEDLKNVFPPEVAVFEPSEAEISHTQKATLVCLATGF |
| | | YPDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSESYQQGVLSATILYEILLGKATLYA |
| | | VLVSALVLMAMVKRKDSRG |
| 53 | R59P2E7 | METLLGLLILWLQLQWVSSKQEVTQIPAALSVPEGENLVLNCS |
| | alpha chain | FTDSAIYNLQWFRQDPGKGLTSLLLIQSSQREQTSGRLNASL |
| | | DKSSGRSTLYIAASQPGDSATYLCAVNSDYKLSFGAGTTVTV |
| | | RANIQNPDPAVYQLRDSKSSDKSVCLFTDFDSQTNVSQSKDS |
| | | DVYITDKTVLDMRSMDFKSNSAVAWSNKSDFACANAFNNSII |
| | | PEDTFFPSPESSCDVKLVEKSFETDTNLNFQNLSVIGFRILLLK |
| | | VAGFNLLMTLRLWSS |
| 54 | R59P2E7 beta | MLSPDLPDSAWNTRLLCHVMLCLLGAVSVAAGVIQSPRHLIK |
| | chain | EKRETATLKCYPIPRHDTVYWYQQGPGQDPQFLISFYEKMQS |
| | | DKGSIPDRFSAQQFSDYHSELNMSSLELGDSALYFCASSLGL |
| | | GTGDYGYTFGSGTRLTVVEDLNKVFPPEVAVFEPSEAEISHT |
| | | QKATLVCLATGFFPDHVELSWWVNGKEVHSGVSTDPQPLKE |
| | | QPALNDSRYCLSSRLRVSATFWQNPRNHFRCQVQFYGLSEN |
| | | DEWTQDRAKPVTQIVSAEAWGRADCGFTSVSYQQGVLSATI |
| | | LYEILLGKATLYAVLVSALVLMAMVKRKDF |
| 55 | R11P3D3 | MEKNPLAAPLLILWFHLDCVSSILNVEQSPQSLHVQEGDSTNF |
| | alpha chain | TCSFPSSNFYALHWYRWETAKSPEALFVMTLNGDEKKKGRIS |
| | | ATLNTKEGYSYLYIKGSQPEDSATYLCALYNNNDMRFGAGTR |
| | | LTVKPNIQNPDPAVYQLRDSKSSDKSVCLFTDFDSQTNVSQS |
| | | KDSDVYITDKTVLDMRSMDFKSNSAVAWSNKSDFACANAFN |
| | | NSIIPEDTFFPSPESSCDVKLVEKSFETDTNLNFQNLSVIGFRIL |
| | | LLKVAGFNLLMTLRLWSS |
| 56 | R11P3D3 beta | MDSWTFCCVSLCILVAKHTDAGVIQSPRHEVTEMGQEVTLRC |
| | chain | KPISGHNSLFWYRQTMMRGLELLIYFNNNVPIDDSGMPEDRF |
| | | SAKMPNASFSTLKIQPSEPRDSAVYFCASSPGSTDTQYFGPG |
| | | TRLTVLEDLKNVFPPEVAVFEPSEAEISHTQKATLVCLATGFY |
| | | PDHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 57 | R16P1C10 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAAVISNFGNEKLTFGT |
| | | GTRLTIIPNIQNPDPAVYQLRDSKSSDKSVCLFTDFDSQTNVS |
| | | QSKDSDVYITDKTVLDMRSMDFKSNSAVAWSNKSDFACANA |
| | | FNNSIIPEDTFFPSPESSCDVKLVEKSFETDTNLNFQNLSVIGF |
| | | RILLLKVAGFNLLMTLRLWSS |
| 58 | R16P1C10 | MGSRLLCWVLLCLLGAGPVKAGVTQTPRYLIKTRGQQVTLSC |
| | beta chain | SPISGHRSVSWYQQTPGQGLQFLFEYFSETQRNKGNFPGRF |
| | | SGRQFSNSRSEMNVSTLELGDSALYLCASSPWDSPNEQYFG |
| | | PGTRLTVTEDLKNVFPPEVAVFEPSEAEISHTQKATLVCLATG |
| | | FYPDHVELSWWVNGKEVHSGVSTDPQPLKEQPALNDSRYCL |
| | | SSRLRVSATFWQNPRNHFRCQVQFYGLSENDEWTQDRAKP |
| | | VTQIVSAEAWGRADCGFTSESYQQGVLSATILYEILLGKATLY |
| | | AVLVSALVLMAMVKRKDSRG |
| 59 | R16P1E8 | MMKSLRVLLVILWLQLSWVWSQQKEVEQDPGPLSVPEGAIV |
| | alpha chain | SLNCTYSNSAFQYFMWYRQYSRKGPELLMYTYSSGNKEDG |
| | | RFTAQVDKSSKYISLFIRDSQPSDSATYLCAMSEAAGNKLTFG |
| | | GGTRVLVKPNIQNPDPAVYQLRDSKSSDKSVCLFTDFDSQTN |
| | | VSQSKDSDVYITDKTVLDMRSMDFKSNSAVAWSNKSDFACA |
| | | NAFNNSIIPEDTFFPSPESSCDVKLVEKSFETDTNLNFQNLSVI |
| | | GFRILLLKVAGFNLLMTLRLWSS |
| 60 | R16P1E8 beta | MGTRLLCWAALCLLGAELTEAGVAQSPRYKIIEKRQSVAFWC |
| | chain | NPISGHATLYWYQQILGQGPKLLIQFQNNGVVDDSQLPKDRF |
| | | SAERLKGVDSTLKIQPAKLEDSAVYLCASSYTNQGEAFFGQG |
| | | TRLTVVEDLNKVFPPEVAVFEPSEAEISHTQKATLVCLATGFF |
| | | PDHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSVSYQQGVLSATILYEILLGKATLYAV |
| | | LVSALVLMAMVKRKDF |
| 61 | R17P1A9 | MKSLRVLLVILWLQLSWVWSQQKEVEQNSGPLSVPEGAIASL |
| | alpha chain | NCTYSDRGSQSFFWYRQYSGKSPELIMSIYSNGDKEDGRFT |
| | | AQLNKASQYVSLLIRDSQPSDSATYLCAVLNQAGTALIFGKGT |
| | | TLSVSSNIQNPDPAVYQLRDSKSSDKSVCLFTDFDSQTNVSQ |
| | | SKDSDVYITDKTVLDMRSMDFKSNSAVAWSNKSDFACANAF |
| | | NNSIIPEDTFFPSPESSCDVKLVEKSFETDTNLNFQNLSVIGFR |
| | | ILLLKVAGFNLLMTLRLWSS |
| 62 | R17P1A9 beta | MGFRLLCCVAFCLLGAGPVDSGVTQTPKHLITATGQRVTLRC |
| | chain | SPRSGDLSVYWYQQSLDQGLQFLIQYYNGEERAKGNILERFS |
| | | AQQFPDLHSELNLSSLELGDSALYFCASSAETGPWLGNEQFF |
| | | GPGTRLTVLEDLKNVFPPEVAVFEPSEAEISHTQKATLVCLAT |
| | | GFYPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSESYQQGVLSATILYEILLGKAT |
| | | LYAVLVSALVLMAMVKRKDSRG |
| 63 | R17P1D7 | MACPGFLWALVISTCLEFSMAQTVTQSQPEMSVQEAETVTLS |
| | alpha chain | CTYDTSESDYYLFWYKQPPSRQMILVIRQEAYKQQNATENRF |
| | | SVNFQKAAKSFSLKISDSQLGDAAMYFCAYRWAQGGSEKLV |
| | | FGKGTKLTVNPYIQKPDPAVYQLRDSKSSDKSVCLFTDFDSQ |
| | | TNVSQSKDSDVYITDKTVLDMRSMDFKSNSAVAWSNKSDFA |
| | | CANAFNNSIIPEDTFFPSPESSCDVKLVEKSFETDTNLNFQNL |
| | | SVIGFRILLLKVAGFNLLMTLRLWSS |
| 64 | R17P1D7 beta | MTIRLLCYMGFYFLGAGLMEADIYQTPRYLVIGTGKKITLECSQ |
| | chain | TMGHDKMYWYQQDPGMELHLIHYSYGVNSTEKGDLSSESTV |
| | | SRIRTEHFPLTLESARPSHTSQYLCATELWSSGGTGELFFGE |
| | | GSRLTVLEDLKNVFPPEVAVFEPSEAEISHTQKATLVCLATGF |
| | | YPDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSESYQQGVLSATILYEILLGKATLYA |
| | | VLVSALVLMAMVKRKDSRG |
| 65 | R17P1G3 | IMSIYSNGDKEDGRFTAQLNKASQYVSLLIRDSQPSDSATYLC |
| | alpha chain | AVGPSGTYKYIFGTGTRLKVLANIQNPDPAVYQLRDSKSSDK |
| | | SVCLFTDFDSQTNVSQSKDSDVYITDKTVLDMRSMDFKSNSA |
| | | VAWSNKSDFACANAFNNSIIPEDTFFPSPESSCDVKLVEKSFE |
| | | TDTNLNFQNLSVIGFRILLLKVAGFNLLMTLRLWSS |
| 66 | R17P1G3 | MGPQLLGYVVLCLLGAGPLEAQVTQNPRYLITVTGKKLTVTC |
| | beta chain | SQNMNHEYMSWYRQDPGLGLRQIYYSMNVEVTDKGDVPEG |
| | | YKVSRKEKRNFPLILESPSPNQTSLYFCASSPGGSGNEQFFG |
| | | PGTRLTVLEDLKNVFPPEVAVFEPSEAEISHTQKATLVCLATG |
| | | FYPDHVELSWWVNGKEVHSGVSTDPQPLKEQPALNDSRYCL |
| | | SSRLRVSATFWQNPRNHFRCQVQFYGLSENDEWTQDRAKP |
| | | VTQIVSAEAWGRADCGFTSESYQQGVLSATILYEILLGKATLY |
| | | AVLVSALVLMAMVKRKDSRG |
| 67 | R17P2B6 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAVVSGGGADGLTFGK |
| | | GTHLIIQPYIQKPDPAVYQLRDSKSSDKSVCLFTDFDSQTNVS |
| | | QSKDSDVYITDKTVLDMRSMDFKSNSAVAWSNKSDFACANA |
| | | FNNSIIPEDTFFPSPESSCDVKLVEKSFETDTNLNFQNLSVIGF |
| | | RILLLKVAGFNLLMTLRLWSS |
| 68 | R17P2B6 beta | MLSPDLPDSAWNTRLLCHVMLCLLGAVSVAAGVIQSPRHLIK |
| | chain | EKRETATLKCYPIPRHDTVYWYQQGPGQDPQFLISFYEKMQS |
| | | DKGSIPDRFSAQQFSDYHSELNMSSLELGDSALYFCASSLGR |
| | | GGQPQHFGDGTRLSILEDLNKVFPPEVAVFEPSEAEISHTQK |
| | | ATLVCLATGFFPDHVELSWWVNGKEVHSGVSTDPQPLKEQP |
| | | ALNDSRYCLSSRLRVSATFWQNPRNHFRCQVQFYGLSENDE |
| | | WTQDRAKPVTQIVSAEAWGRADCGFTSVSYQQGVLSATILYE |
| | | ILLGKATLYAVLVSALVLMAMVKRKDF |
| 69 | R11P3D3KE | MEKNPLAAPLLILWFHLDCVSSILNVEQSPQSLHVQEGDSTNF |
| | alpha chain | TCSFPSSNFYALHWYRKETAKSPEALFVMTLNGDEKKKGRIS |
| | | ATLNTKEGYSYLYIKGSQPEDSATYLCALYNNNDMRFGAGTR |
| | | LTVKPNIQNPDPAVYQLRDSKSSDKSVCLFTDFDSQTNVSQS |
| | | KDSDVYITDKTVLDMRSMDFKSNSAVAWSNKSDFACANAFN |
| | | NSIIPEDTFFPSPESSCDVKLVEKSFETDTNLNFQNLSVIGFRIL |
| | | LLKVAGFNLLMTLRLWSS |
| 70 | R11P3D3KE | NNNVPIDDSGMPEDRFSAKMPNASFSTLKIQPSEPRDSAVYF |
| | beta chain | CASSPGSTDTQYFGPGTRLTVLEDLKNVFPPEVAVFEPSEAEI |
| | | SHTQKATLVCLATGFYPDHVELSWWVNGKEVHSGVSTDPQP |
| | | LKEQPALNDSRYCLSSRLRVSATFWQNPRNHFRCQVQFYGL |
| | | SENDEWTQDRAKPVTQIVSAEAWGRADCGFTSESYQQGVLS |
| | | ATILYEILLGKATLYAVLVSALVLMAMVKRKDSRG |
| 71 | R39P1C12 | TYLYWYKQEPGAGLQLLTYIFSNMDMKQDQRLTVLLNKKDKH |
| | alpha chain | LSLRIADTQTGDSAIYFCAEIDNQGGKLIFGQGTELSVKPNIQN |
| | | PDPAVYQLRDSKSSDKSVCLFTDFDSQTNVSQSKDSDVYITD |
| | | KTVLDMRSMDFKSNSAVAWSNKSDFACANAFNNSIIPEDTFF |
| | | PSPESSCDVKLVEKSFETDTNLNFQNLSVIGFRILLLKVAGFNL |
| | | LMTLRLWSS |
| 72 | R39P1C12 | MGPGLLCWALLCLLGAGLVDAGVTQSPTHLIKTRGQQVTLRC |
| | beta chain | SPKSGHDTVSWYQQALGQGPQFIFQYYEEEERQRGNFPDRF |
| | | SGHQFPNYSSELNVNALLLGDSALYLCASSQLNTEAFFGQGT |
| | | RLTVVEDLNKVFPPEVAVFEPSEAEISHTQKATLVCLATGFFP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSVSYQQGVLSATILYEILLGKATLYAV |
| | | LVSALVLMAMVKRKDF |
| 73 | R39P1F5 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAVNNARLMFGDGTQL |
| | | VVKPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 74 | R39P1F5 beta | MDTWLVCWAIFSLLKAGLTEPEVTQTPSHQVTQMGQEVILRC |
| | chain | VPISNHLYFYWYRQILGQKVEFLVSFYNNEISEKSEIFDDQFSV |
| | | ERPDGSNFTLKIRSTKLEDSAMYFCASSGQGANEQYFGPGT |
| | | RLTVTEDLKNVFPPEVAVFEPSEAEISHTQKATLVCLATGFYP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 75 | R40P1C2 | MACPGFLWALVISTCLEFSMAQTVTQSQPEMSVQEAETVTLS |
| | alpha chain | CTYDTSESDYYLFWYKQPPSRQMILVIRQEAYKQQNATENRF |
| | | SVNFQKAAKSFSLKISDSQLGDAAMYFCAYLNYQLIWGAGTK |
| | | LIIKPDIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 76 | R40P1C2 beta | MDTWLVCWAIFSLLKAGLTEPEVTQTPSHQVTQMGQEVILRC |
| | chain | VPISNHLYFYWYRQILGQKVEFLVSFYNNEISEKSEIFDDQFSV |
| | | ERPDGSNFTLKIRSTKLEDSAMYFCASSEMTAVGQYFGPGTR |
| | | LTVTEDLKNVFPPEVAVFEPSEAEISHTQKATLVCLATGFYPD |
| | | HVELSWWVNGKEVHSGVSTDPQPLKEQPALNDSRYCLSSRL |
| | | RVSATFWQNPRNHFRCQVQFYGLSENDEWTQDRAKPVTQIV |
| | | SAEAWGRADCGFTSESYQQGVLSATILYEILLGKATLYAVLVS |
| | | ALVLMAMVKRKDSRG |
| 77 | R41P3E6 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAAFSGYALNFGKGTS |
| | | LLVTPHIQNPDPAVYQLRDSKSSDKSVCLFTDFDSQTNVSQS |
| | | KDSDVYITDKTVLDMRSMDFKSNSAVAWSNKSDFACANAFN |
| | | NSIIPEDTFFPSPESSCDVKLVEKSFETDTNLNFQNLSVIGFRIL |
| | | LLKVAGFNLLMTLRLWSS |
| 78 | R41P3E6 beta | MDTWLVCWAIFSLLKAGLTEPEVTQTPSHQVTQMGQEVILRC |
| | chain | VPISNHLYFYWYRQILGQKVEFLVSFYNNEISEKSEIFDDQFSV |
| | | ERPDGSNFTLKIRSTKLEDSAMYFCASSQYTGELFFGEGSRL |
| | | TVLEDLKNVFPPEVAVFEPSEAEISHTQKATLVCLATGFYPDH |
| | | VELSWWVNGKEVHSGVSTDPQPLKEQPALNDSRYCLSSRLR |
| | | VSATFWQNPRNHFRCQVQFYGLSENDEWTQDRAKPVTQIVS |
| | | AEAWGRADCGFTSESYQQGVLSATILYEILLGKATLYAVLVSA |
| | | LVLMAMVKRKDSRG |
| 79 | R43P3G4 | MKSLRVLLVILWLQLSWVWSQQKEVEQNSGPLSVPEGAIASL |
| | alpha chain | NCTYSDRGSQSFFWYRQYSGKSPELIMFIYSNGDKEDGRFT |
| | | AQLNKASQYVSLLIRDSQPSDSATYLCAVNGGDMRFGAGTRL |
| | | TVKPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 80 | R43P3G4 | MDTWLVCWAIFSLLKAGLTEPEVTQTPSHQVTQMGQEVILRC |
| | beta chain | VPISNHLYFYWYRQILGQKVEFLVSFYNNEISEKSEIFDDQFSV |
| | | ERPDGSNFTLKIRSTKLEDSAMYFCASSGQGALEQYFGPGTR |
| | | LTVTEDLKNVFPPEVAVFEPSEAEISHTQKATLVCLATGFYPD |
| | | HVELSWWVNGKEVHSGVSTDPQPLKEQPALNDSRYCLSSRL |
| | | RVSATFWQNPRNHFRCQVQFYGLSENDEWTQDRAKPVTQIV |
| | | SAEAWGRADCGFTSESYQQGVLSATILYEILLGKATLYAVLVS |
| | | ALVLMAMVKRKDSRG |
| 81 | R44P3B3 | MAMLLGASVLILWLQPDWVNSQQKNDDQQVKQNSPSLSVQ |
| | alpha chain | EGRISILNCDYTNSMFDYFLWYKKYPAEGPTFLISISSIKDKNE |
| | | DGRFTVFLNKSAKHLSLHIVPSQPGDSAVYFCAASGLYNQGG |
| | | KLIFGQGTELSVKPNIQNPDPAVYQLRDSKSSDKSVCLFTDFD |
| | | SQTNVSQSKDSDVYITDKTVLDMRSMDFKSNSAVAWSNKSD |
| | | FACANAFNNSIIPEDTFFPSPESSCDVKLVEKSFETDTNLNFQ |
| | | NLSVIGFRILLLKVAGFNLLMTLRLWSS |
| 82 | R44P3B3 beta | MGCRLLCCVVFCLLQAGPLDTAVSQTPKYLVTQMGNDKSIKC |
| | chain | EQNLGHDTMYWYKQDSKKFLKIMFSYNNKELIINETVPNRFSP |
| | | KSPDKAHLNLHINSLELGDSAVYFCASSLGDRGYEQYFGPGT |
| | | RLTVTEDLKNVFPPEVAVFEPSEAEISHTQKATLVCLATGFYP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 83 | R44P3E7 | MKTFAGFSFLFLWLQLDCMSRGEDVEQSLFLSVREGDSSVIN |
| | alpha chain | CTYTDSSSTYLYWYKQEPGAGLQLLTYIFSNMDMKQDQRLTV |
| | | LLNKKDKHLSLRIADTQTGDSAIYFCAEINNNARLMFGDGTQL |
| | | VVKPNIQNPDPAVYQLRDSKSSDKSVCLFTDFDSQTNVSQSK |
| | | DSDVYITDKTVLDMRSMDFKSNSAVAWSNKSDFACANAFNN |
| | | SIIPEDTFFPSPESSCDVKLVEKSFETDTNLNFQNLSVIGFRILL |
| | | LKVAGFNLLMTLRLWSS |
| 84 | R44P3E7 beta | MLSPDLPDSAWNTRLLCHVMLCLLGAVSVAAGVIQSPRHLIK |
| | chain | EKRETATLKCYPIPRHDTVYWYQQGPGQDPQFLISFYEKMQS |
| | | DKGSIPDRFSAQQFSDYHSELNMSSLELGDSALYFCASSPPD |
| | | QNTQYFGPGTRLTVLEDLKNVFPPEVAVFEPSEAEISHTQKAT |
| | | LVCLATGFYPDHVELSWWVNGKEVHSGVSTDPQPLKEQPAL |
| | | NDSRYCLSSRLRVSATFWQNPRNHFRCQVQFYGLSENDEW |
| | | TQDRAKPVTQIVSAEAWGRADCGFTSESYQQGVLSATILYEIL |
| | | LGKATLYAVLVSALVLMAMVKRKDSRG |
| 85 | R49P2B7 | MLLLLVPVLEVIFTLGGTRAQSVTQLGSHVSVSEGALVLLRCN |
| | alpha chain | YSSSVPPYLFWYVQYPNQGLQLLLKYTTGATLVKGINGFEAE |
| | | FKKSETSFHLTKPSAHMSDAAEYFCAVRIFGNEKLTFGTGTRL |
| | | TIIPNIQNPDPAVYQLRDSKSSDKSVCLFTDFDSQTNVSQSKD |
| | | SDVYITDKTVLDMRSMDFKSNSAVAWSNKSDFACANAFNNSI |
| | | IPEDTFFPSPESSCDVKLVEKSFETDTNLNFQNLSVIGFRILLL |
| | | KVAGFNLLMTLRLWSS |
| 86 | R49P2B7 beta | MGIRLLCRVAFCFLAVGLVDVKVTQSSRYLVKRTGEKVFLEC |
| | chain | VQDMDHENMFWYRQDPGLGLRLIYFSYDVKMKEKGDIPEGY |
| | | SVSREKKERFSLILESASTNQTSMYLCASSLMGELTGELFFGE |
| | | GSRLTVLEDLKNVFPPEVAVFEPSEAEISHTQKATLVCLATGF |
| | | YPDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSESYQQGVLSATILYEILLGKATLYA |
| | | VLVSALVLMAMVKRKDSRG |
| 87 | R55P1G7 | MMKSLRVLLVILWLQLSWVWSQQKEVEQDPGPLSVPEGAIV |
| | alpha chain | SLNCTYSNSAFQYFMWYRQYSRKGPELLMYTYSSGNKEDG |
| | | RFTAQVDKSSKYISLFIRDSQPSDSATYLCAMMGDTGTASKLT |
| | | FGTGTRLQVTLDIQNPDPAVYQLRDSKSSDKSVCLFTDFDSQ |
| | | TNVSQSKDSDVYITDKTVLDMRSMDFKSNSAVAWSNKSDFA |
| | | CANAFNNSIIPEDTFFPSPESSCDVKLVEKSFETDTNLNFQNL |
| | | SVIGFRILLLKVAGFNLLMTLRLWSS |
| 88 | R55P1G7 | MGIRLLCRVAFCFLAVGLVDVKVTQSSRYLVKRTGEKVFLEC |
| | beta chain | VQDMDHENMFWYRQDPGLGLRLIYFSYDVKMKEKGDIPEGY |
| | | SVSREKKERFSLILESASTNQTSMYLCASSFGGYEQYFGPGT |
| | | RLTVTEDLKNVFPPEVAVFEPSEAEISHTQKATLVCLATGFYP |
| | | DHVELSWWVNGKEVHSGVSTDPQPLKEQPALNDSRYCLSS |
| | | RLRVSATFWQNPRNHFRCQVQFYGLSENDEWTQDRAKPVT |
| | | QIVSAEAWGRADCGFTSESYQQGVLSATILYEILLGKATLYAV |
| | | LVSALVLMAMVKRKDSRG |
| 89 | R59P2A7 | VKPNIQNPDPAVYQLRDSKSSDKSVCLFTDFDSQTNVSQSKD |
| | alpha chain | SDVYITDKTVLDMRSMDFKSNSAVAWSNKSDFACANAFNNSI |
| | | IPEDTFFPSPESSCDVKLVEKSFETDTNLNFQNLSVIGFRILLL |
| | | KVAGFNLLMTLRLWSS |
| 90 | R59P2A7 beta | MLCSLLALLLGTFFGVRSQTIHQWPATLVQPVGSPLSLECTVE |
| | chain | GTSNPNLYWYRQAAGRGLQLLFYSVGIGQISSEVPQNLSASR |
| | | PQDRQFILSSKKLLLSDSGFYLCAWSGLVAEQFFGPGTRLTV |
| | | LEDLKNVFPPEVAVFEPSEAEISHTQKATLVCLATGFYPDHVE |
| | | LSWWVNGKEVHSGVSTDPQPLKEQPALNDSRYCLSSRLRVS |
| | | ATFWQNPRNHFRCQVQFYGLSENDEWTQDRAKPVTQIVSAE |
| | | AWGRADCGFTSESYQQGVLSATILYEILLGKATLYAVLVSALV |
| | | LMAMVKRKDSRG |
| 91 | PTE WPRE | tgaaagaccccacctgtaggtttggcaagctagcttaagtaacgccattttgcaaggca |
| | | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatggactctt |
| | | ggaccttctgctgcgtgagcctgtgcatcctggtggccaagcacacagacgccggcgt |
| | | gatccagtcccctaggcacgaggtgaccgagatgggccaggaggtgacactgcgct |
| | | gtaagccaatctctggccacaacagcctgttttggtatagggagaccatgatgcgcgg |
| | | cctggagctgctgatctacttcaataacaatgtgcccatcgacgattccggcatgcctga |
| | | ggatcggttttctgccaagatgcccaatgccagcttctccacactgaagatccagccta |
| | | gcgagccaagagactccgccgtgtatttttgcgcctctagcccaggcagcaccgatac |
| | | acagtacttcggaccaggaaccaggctgacagtgctggaggacctgaagaacgtgtt |
| | | cccccctgaggtggccgtgtttgagccctctgaggccgagatcagccacacccagaa |
| | | ggccaccctggtgtgcctggcaaccggcttctatcctgatcacgtggagctgtcctggtg |
| | | ggtgaacggcaaggaggtgcacagcggcgtgtccacagacccacagcccctgaag |
| | | gagcagccagccctgaatgatagccggtattgcctgtcctctcggctgagagtgtccgc |
| | | caccttttggcagaacccccggaatcacttcagatgtcaggtgcagttttacggcctgtc |
| | | cgagaacgatgagtggacccaggaccgggccaagcctgtgacacagatcgtgtctg |
| | | ccgaggcatggggaagagcagactgtggcttcacctctgagagctaccagcagggc |
| | | gtgctgagcgccaccatcctgtatgagatcctgctgggcaaggccacactgtacgccg |
| | | tcctggtctccgctctggtgctgatggcaatggtcaaaagaaaagatagtcggggacg |
| | | ggccaagagatctggcagcggcgccaccaatttcagcctgctgaaacaggccggcg |
| | | acgtggaagagaaccctggccccatggagaagaatcccctggctgcccccctgctg |
| | | atcctgtggtttcacctggactgcgtgtcctctatcctgaatgtggaacagagcccacag |
| | | agcctgcacgtgcaggagggcgactccaccaacttcacatgctcttttcctagctccaa |
| | | cttctacgccctgcactggtacagaaaggagaccgcaaagtccccagaggccctgtt |
| | | cgtgatgacactgaacggcgatgagaagaagaagggccgcatcagcgccaccctg |
| | | aatacaaaggagggctactcctatctgtacatcaagggctcccagcctgaggactctg |
| | | ccacctatctgtgcgccctgtacaacaataacgatatgcggtttggcgccggcaccag |
| | | actgacagtgaagccaaacatccagaatccagaccccgccgtgtatcagctgcggg |
| | | acagcaagtctagcgataagagcgtgtgcctgttcaccgactttgattctcagacaaac |
| | | gtgagccagtccaaggacagcgacgtgtacatcaccgacaagacagtgctggatat |
| | | gagaagcatggacttcaagtctaacagcgccgtggcctggtccaataagtctgatttcg |
| | | cctgcgccaatgcctttaataactccatcatccccgaggataccttctttccttctccagag |
| | | tcctcttgtgacgtgaagctggtggagaagtctttcgagaccgatacaaacctgaattttc |
| | | agaacctgagcgtgatcggcttcaggatcctgctgctgaaggtggccggctttaatctg |
| | | ctgatgaccctgaggctgtggagctcccgggccaagagatctggcagcggcgaggg |
| | | cagaggcagcctgctgacctgcggcgacgtggaggagaaccccggccccatgcgc |
| | | ccgagactgtggcttctgctcgccgcgcaactgactgtcctgcacggaaacagcgtgc |
| | | tgcagcagacaccggcctacatcaaagtgcagaccaacaagatggtcatgctgtcct |
| | | gcgaggccaagatttccctctccaacatgcggatctattggttgcggcagagacaggc |
| | | gccttcctcggactcccaccatgagttcttggccctgtgggactccgccaagggaactat |
| | | tcacggcgaagaagtggaacaggagaagatcgccgtgtttcgcgatgcctcccgcttt |
| | | atactgaatctgacctccgtgaagcccgaagatagcgggatctacttttgcatgattgtg |
| | | ggctcacccgaactgaccttcgggaagggcactcagctgagcgtggtggacttcctcc |
| | | ccactaccgcccaacccactaagaagtcaaccctgaagaagcgggtttgcagactcc |
| | | cacggccggaaacgcagaagggtccgctgtgttccccgatcaccctggggctccttgt |
| | | ggctggagtgctggtccttctggtgtcccttggcgtcgccattcacctctgctgccggaga |
| | | aggagggccagactgaggttcatgaagcagcctcagggagaggggatcagtggca |
| | | ctttcgtgccacaatgcctccatggctactattccaacaccaccacctcgcaaaagctg |
| | | ctgaacccctggatcctgaaaacccgggccaagagatctggcagcggccagtgcac |
| | | caactacgccctgctgaagctggccggcgacgtggagagcaaccccggccccatgg |
| | | cgcttcccgtgaccgcactcctgttgccccttgccctgctgttgcacgccgcacgaccttc |
| | | ccaattccgggtgtcccctctggatcgcacctggaacctcggggaaacggtggagctc |
| | | aagtgtcaagtcctcctgtcgaacccgaccagcggatgcagctggctgttccagccga |
| | | gaggagctgccgcctcacccaccttcctcctgtacttgagccagaacaagccgaagg |
| | | ccgctgagggtctggacacccagcgcttctcgggcaaacggctgggagacacttttgt |
| | | gctgactctctccgacttccggcgggagaacgagggctactacttctgctctgcgctctc |
| | | caattcaatcatgtacttctcacacttcgtgccggtgttcctgcctgccaagcccaccact |
| | | actccggcacccagacctccaactcccgctcccaccatcgcgtcccaaccccMcgct |
| | | gcgccctgaagcgtgtcggcctgctgctggaggagccgtgcatacccgcggtctgga |
| | | cttcgcgtgcgacatctacatttgggcccctttggctggcacctgtggagtgctgctcctgt |
| | | cccttgtgatcaccctgtactgcaaccaccggaataggcggagagtctgcaagtgtcc |
| | | gcggcctgtcgtgaagtcaggagataagccgagcctgtccgcacgctacgtgtgaac |
| | | cggtccgcagtctgacgtacgcgtaatcaacctctggattacaaaatttgtgaaagattg |
| | | actggtattcttaactatgttgctccttttacgctatgtggatacgctgctttaatgcctttgtat |
| | | catgctattgcttcccgtatggctttcattttctcctccttgtataaatcctggttgctgtctcttta |
| | | tgaggagttgtggcccgttgtcaggcaacgtggcgtggtgtgcactgtgtttgctgacgc |
| | | aacccccactggttggggcattgccaccacctgtcagctcctttccgggactttcgctttc |
| | | cccctccctattgccacggcggaactcatcgccgcctgccttgcccgctgctggacagg |
| | | ggctcggctgttgggcactgacaattccgtggtgttgtcggggaagctgacgtcctttcc |
| | | atggctgctcgcctgtgttgccacctggattctgcgcgggacgtccttctgctacgtccctt |
| | | cggccctcaatccagcggaccttccttcccgcggcctgctgccggctctgcggcctcttc |
| | | cgcgtcttcgccttcgccctcagacgagtcggatctccctttgggccgcctccccgcc |
| 92 | TPE WPRE | tgaaagaccccacctgtaggtttggcaagctagcttaagtaacgccattttgcaaggca |
| | | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatggactctt |
| | | ggaccttctgctgcgtgagcctgtgcatcctggtggccaagcacacagacgccggcgt |
| | | gatccagtcccctaggcacgaggtgaccgagatgggccaggaggtgacactgcgct |
| | | gtaagccaatctctggccacaacagcctgttttggtatagggagaccatgatgcgcgg |
| | | cctggagctgctgatctacttcaataacaatgtgcccatcgacgattccggcatgcctga |
| | | ggatcggttttctgccaagatgcccaatgccagcttctccacactgaagatccagccta |
| | | gcgagccaagagactccgccgtgtatttttgcgcctctagcccaggcagcaccgatac |
| | | acagtacttcggaccaggaaccaggctgacagtgctggaggacctgaagaacgtgtt |
| | | cccccctgaggtggccgtgtttgagccctctgaggccgagatcagccacacccagaa |
| | | ggccaccctggtgtgcctggcaaccggcttctatcctgatcacgtggagctgtcctggtg |
| | | ggtgaacggcaaggaggtgcacagcggcgtgtccacagacccacagcccctgaag |
| | | gagcagccagccctgaatgatagccggtattgcctgtcctctcggctgagagtgtccgc |
| | | caccttttggcagaacccccggaatcacttcagatgtcaggtgcagttttacggcctgtc |
| | | cgagaacgatgagtggacccaggaccgggccaagcctgtgacacagatcgtgtctg |
| | | ccgaggcatggggaagagcagactgtggcttcacctctgagagctaccagcagggc |
| | | gtgctgagcgccaccatcctgtatgagatcctgctgggcaaggccacactgtacgccg |
| | | tcctggtctccgctctggtgctgatggcaatggtcaaaagaaaagatagtcggggacg |
| | | ggccaagagatctggcagcggcgagggcagaggcagcctgctgacctgcggcgac |
| | | gtggaggagaaccccggccccatggagaagaatcccctggctgcccccctgctgatc |
| | | ctgtggtttcacctggactgcgtgtcctctatcctgaatgtggaacagagcccacagagc |
| | | ctgcacgtgcaggagggcgactccaccaacttcacatgctcttttcctagctccaacttct |
| | | acgccctgcactggtacagaaaggagaccgcaaagtccccagaggccctgttcgtg |
| | | atgacactgaacggcgatgagaagaagaagggccgcatcagcgccaccctgaata |
| | | caaaggagggctactcctatctgtacatcaagggctcccagcctgaggactctgccac |
| | | ctatctgtgcgccctgtacaacaataacgatatgcggtttggcgccggcaccagactga |
| | | cagtgaagccaaacatccagaatccagaccccgccgtgtatcagctgcgggacagc |
| | | aagtctagcgataagagcgtgtgcctgttcaccgactttgattctcagacaaacgtgag |
| | | ccagtccaaggacagcgacgtgtacatcaccgacaagacagtgctggatatgagaa |
| | | gcatggacttcaagtctaacagcgccgtggcctggtccaataagtctgatttcgcctgcg |
| | | ccaatgcctttaataactccatcatccccgaggataccttctttccttctccagagtcctctt |
| | | gtgacgtgaagctggtggagaagtctttcgagaccgatacaaacctgaattttcagaac |
| | | ctgagcgtgatcggcttcaggatcctgctgctgaaggtggccggctttaatctgctgatg |
| | | accctgaggctgtggagctcccgggccaagagatctggcagcggcgccaccaatttc |
| | | agcctgctgaaacaggccggcgacgtggaagagaaccctggccccatgcgcccga |
| | | gactgtggcttctgctcgccgcgcaactgactgtcctgcacggaaacagcgtgctgca |
| | | gcagacaccggcctacatcaaagtgcagaccaacaagatggtcatgctgtcctgcga |
| | | ggccaagatttccctctccaacatgcggatctattggttgcggcagagacaggcgcctt |
| | | cctcggactcccaccatgagttcttggccctgtgggactccgccaagggaactattcac |
| | | ggcgaagaagtggaacaggagaagatcgccgtgtttcgcgatgcctcccgctttatac |
| | | tgaatctgacctccgtgaagcccgaagatagcgggatctacttttgcatgattgtgggct |
| | | cacccgaactgaccttcgggaagggcactcagctgagcgtggtggacttcctccccac |
| | | taccgcccaacccactaagaagtcaaccctgaagaagcgggtttgcagactcccac |
| | | ggccggaaacgcagaagggtccgctgtgttccccgatcaccctggggctccttgtggc |
| | | tggagtgctggtccttctggtgtcccttggcgtcgccattcacctctgctgccggagaagg |
| | | agggccagactgaggttcatgaagcagcctcagggagaggggatcagtggcactttc |
| | | gtgccacaatgcctccatggctactattccaacaccaccacctcgcaaaagctgctga |
| | | acccctggatcctgaaaacccgggccaagagatctggcagcggccagtgcaccaa |
| | | ctacgccctgctgaagctggccggcgacgtggagagcaaccccggccccatggcgc |
| | | ttcccgtgaccgcactcctgttgccccttgccctgctgttgcacgccgcacgaccttccca |
| | | attccgggtgtcccctctggatcgcacctggaacctcggggaaacggtggagctcaag |
| | | tgtcaagtcctcctgtcgaacccgaccagcggatgcagctggctgttccagccgagag |
| | | gagctgccgcctcacccaccttcctcctgtacttgagccagaacaagccgaaggccg |
| | | ctgagggtctggacacccagcgcttctcgggcaaacggctgggagacacttttgtgctg |
| | | actctctccgacttccggcgggagaacgagggctactacttctgctctgcgctctccaatt |
| | | caatcatgtacttctcacacttcgtgccggtgttcctgcctgccaagcccaccactactcc |
| | | ggcacccagacctccaactcccgctcccaccatcgcgtcccaacccctttcgctgcgc |
| | | cctgaagcgtgtcggcctgctgctggaggagccgtgcatacccgcggtctggacttcg |
| | | cgtgcgacatctacatttgggcccctttggctggcacctgtggagtgctgctcctgtccctt |
| | | gtgatcaccctgtactgcaaccaccggaataggcggagagtctgcaagtgtccgcgg |
| | | cctgtcgtgaagtcaggagataagccgagcctgtccgcacgctacgtgtgaaccggtc |
| | | cgcagtctgacgtacgcgtaatcaacctctggattacaaaatttgtgaaagattgactgg |
| | | tattcttaactatgttgctccttttacgctatgtggatacgctgctttaatgcctttgtatcatgct |
| | | attgcttcccgtatggctttcattttctcctccttgtataaatcctggttgctgtctctttatgagg |
| | | agttgtggcccgttgtcaggcaacgtggcgtggtgtgcactgtgtttgctgacgcaaccc |
| | | ccactggttggggcattgccaccacctgtcagctcctttccgggactttcgctttccccctc |
| | | cctattgccacggcggaactcatcgccgcctgccttgcccgctgctggacaggggctc |
| | | ggctgttgggcactgacaattccgtggtgttgtcggggaagctgacgtcctttccatggct |
| | | gctcgcctgtgttgccacctggattctgcgcgggacgtccttctgctacgtcccttcggcc |
| | | ctcaatccagcggaccttccttcccgcggcctgctgccggctctgcggcctcttccgcgt |
| | | cttcgccttcgccctcagacgagtcggatctccctttgggccgcctccccgcc |
| 93 | PTE fn WPRE | tgaaagaccccacctgtaggtttggcaagctagcttaagtaacgccattttgcaaggca |
| | | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatggactctt |
| | | ggaccttctgctgcgtgagcctgtgcatcctggtggccaagcacacagacgccggcgt |
| | | gatccagtcccctaggcacgaggtgaccgagatgggccaggaggtgacactgcgct |
| | | gtaagccaatctctggccacaacagcctgttttggtatagggagaccatgatgcgcgg |
| | | cctggagctgctgatctacttcaataacaatgtgcccatcgacgattccggcatgcctga |
| | | ggatcggttttctgccaagatgcccaatgccagcttctccacactgaagatccagccta |
| | | gcgagccaagagactccgccgtgtatttttgcgcctctagcccaggcagcaccgatac |
| | | acagtacttcggaccaggaaccaggctgacagtgctggaggacctgaagaacgtgtt |
| | | cccccctgaggtggccgtgtttgagccctctgaggccgagatcagccacacccagaa |
| | | ggccaccctggtgtgcctggcaaccggcttctatcctgatcacgtggagctgtcctggtg |
| | | ggtgaacggcaaggaggtgcacagcggcgtgtccacagacccacagcccctgaag |
| | | gagcagccagccctgaatgatagccggtattgcctgtcctctcggctgagagtgtccgc |
| | | caccttttggcagaacccccggaatcacttcagatgtcaggtgcagttttacggcctgtc |
| | | cgagaacgatgagtggacccaggaccgggccaagcctgtgacacagatcgtgtctg |
| | | ccgaggcatggggaagagcagactgtggcttcacctctgagagctaccagcagggc |
| | | gtgctgagcgccaccatcctgtatgagatcctgctgggcaaggccacactgtacgccg |
| | | tcctggtctccgctctggtgctgatggcaatggtcaaaagaaaagatagtcggggatct |
| | | ggcagcggcgccaccaatttcagcctgctgaaacaggccggcgacgtggaagaga |
| | | accctggccccatggagaagaatcccctggctgcccccctgctgatcctgtggtttcac |
| | | ctggactgcgtgtcctctatcctgaatgtggaacagagcccacagagcctgcacgtgc |
| | | aggagggcgactccaccaacttcacatgctcttttcctagctccaacttctacgccctgc |
| | | actggtacagaaaggagaccgcaaagtccccagaggccctgttcgtgatgacactg |
| | | aacggcgatgagaagaagaagggccgcatcagcgccaccctgaatacaaaggag |
| | | ggctactcctatctgtacatcaagggctcccagcctgaggactctgccacctatctgtgc |
| | | gccctgtacaacaataacgatatgcggtttggcgccggcaccagactgacagtgaag |
| | | ccaaacatccagaatccagaccccgccgtgtatcagctgcgggacagcaagtctag |
| | | cgataagagcgtgtgcctgttcaccgactttgattctcagacaaacgtgagccagtcca |
| | | aggacagcgacgtgtacatcaccgacaagacagtgctggatatgagaagcatggac |
| | | ttcaagtctaacagcgccgtggcctggtccaataagtctgatttcgcctgcgccaatgcc |
| | | tttaataactccatcatccccgaggataccttctttccttctccagagtcctcttgtgacgtga |
| | | agctggtggagaagtctttcgagaccgatacaaacctgaattttcagaacctgagcgtg |
| | | atcggcttcaggatcctgctgctgaaggtggccggctttaatctgctgatgaccctgagg |
| | | ctgtggagctcccgggccaagagaggcagcggcgagggcagaggcagcctgctg |
| | | acctgcggcgacgtggaggagaaccccggccccatgcgcccgagactgtggcttct |
| | | gctcgccgcgcaactgactgtcctgcacggaaacagcgtgctgcagcagacaccgg |
| | | cctacatcaaagtgcagaccaacaagatggtcatgctgtcctgcgaggccaagatttc |
| | | cctctccaacatgcggatctattggttgcggcagagacaggcgccttcctcggactccc |
| | | accatgagttcttggccctgtgggactccgccaagggaactattcacggcgaagaagt |
| | | ggaacaggagaagatcgccgtgtttcgcgatgcctcccgctttatactgaatctgacctc |
| | | cgtgaagcccgaagatagcgggatctacttttgcatgattgtgggctcacccgaactga |
| | | ccttcgggaagggcactcagctgagcgtggtggacttcctccccactaccgcccaacc |
| | | cactaagaagtcaaccctgaagaagcgggtttgcagactcccacggccggaaacgc |
| | | agaagggtccgctgtgttccccgatcaccctggggctccttgtggctggagtgctggtcc |
| | | ttctggtgtcccttggcgtcgccattcacctctgctgccggagaaggagggccagactg |
| | | aggttcatgaagcagcctcagggagaggggatcagtggcactttcgtgccacaatgc |
| | | ctccatggctactattccaacaccaccacctcgcaaaagctgctgaacccctggatcct |
| | | gaaaacccgggccaagagatctggcagcggccagtgcaccaactacgccctgctg |
| | | aagctggccggcgacgtggagagcaaccccggccccatggcgcttcccgtgaccgc |
| | | actcctgttgccccttgccctgctgttgcacgccgcacgaccttcccaattccgggtgtcc |
| | | cctctggatcgcacctggaacctcggggaaacggtggagctcaagtgtcaagtcctcc |
| | | tgtcgaacccgaccagcggatgcagctggctgttccagccgagaggagctgccgcct |
| | | cacccaccttcctcctgtacttgagccagaacaagccgaaggccgctgagggtctgg |
| | | acacccagcgcttctcgggcaaacggctgggagacacttttgtgctgactctctccgact |
| | | tccggcgggagaacgagggctactacttctgctctgcgctctccaattcaatcatgtactt |
| | | ctcacacttcgtgccggtgttcctgcctgccaagcccaccactactccggcacccagac |
| | | ctccaactcccgctcccaccatcgcgtcccaacccctttcgctgcgccctgaagcgtgtc |
| | | ggcctgctgctggaggagccgtgcatacccgcggtctggacttcgcgtgcgacatcta |
| | | catttgggcccctttggctggcacctgtggagtgctgctcctgtcccttgtgatcaccctgta |
| | | ctgcaaccaccggaataggcggagagtctgcaagtgtccgcggcctgtcgtgaagtc |
| | | aggagataagccgagcctgtccgcacgctacgtgtgaaccggtccgcagtctgacgt |
| | | acgcgtaatcaacctctggattacaaaatttgtgaaagattgactggtattcttaactatgt |
| | | tgctccttttacgctatgtggatacgctgctttaatgcctttgtatcatgctattgcttcccgtat |
| | | ggctttcattttctcctccttgtataaatcctggttgctgtctctttatgaggagttgtggcccgtt |
| | | gtcaggcaacgtggcgtggtgtgcactgtgtttgctgacgcaacccccactggttgggg |
| | | cattgccaccacctgtcagctcctttccgggactttcgctttccccctccctattgccacgg |
| | | cggaactcatcgccgcctgccttgcccgctgctggacaggggctcggctgttgggcact |
| | | gacaattccgtggtgttgtcggggaagctgacgtcctttccatggctgctcgcctgtgttgc |
| | | cacctggattctgcgcgggacgtccttctgctacgtcccttcggccctcaatccagcgga |
| | | ccttccttcccgcggcctgctgccggctctgcggcctcttccgcgtcttcgccttcgccctc |
| | | agacgagtcggatctccctttgggccgcctccccgcc |
| 94 | PTE CD8 TCR | tgaaagaccccacctgtaggtttggcaagctagcttaagtaacgccattttgcaaggca |
| | WPRE | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatgcgcccg |
| | | agactgtggcttctgctcgccgcgcaactgactgtcctgcacggaaacagcgtgctgc |
| | | agcagacaccggcctacatcaaagtgcagaccaacaagatggtcatgctgtcctgcg |
| | | aggccaagatttccctctccaacatgcggatctattggttgcggcagagacaggcgcct |
| | | tcctcggactcccaccatgagttcttggccctgtgggactccgccaagggaactattcac |
| | | ggcgaagaagtggaacaggagaagatcgccgtgtttcgcgatgcctcccgctttatac |
| | | tgaatctgacctccgtgaagcccgaagatagcgggatctacttttgcatgattgtgggct |
| | | cacccgaactgaccttcgggaagggcactcagctgagcgtggtggacttcctccccac |
| | | taccgcccaacccactaagaagtcaaccctgaagaagcgggtttgcagactcccac |
| | | ggccggaaacgcagaagggtccgctgtgttccccgatcaccctggggctccttgtggc |
| | | tggagtgctggtccttctggtgtcccttggcgtcgccattcacctctgctgccggagaagg |
| | | agggccagactgaggttcatgaagcagcctcagggagaggggatcagtggcactttc |
| | | gtgccacaatgcctccatggctactattccaacaccaccacctcgcaaaagctgctga |
| | | acccctggatcctgaaaacccgggccaagagatctggcagcggcgccaccaatttc |
| | | agcctgctgaaacaggccggcgacgtggaagagaaccctggccccatggcgcttcc |
| | | cgtgaccgcactcctgttgccccttgccctgctgttgcacgccgcacgaccttcccaattc |
| | | cgggtgtcccctctggatcgcacctggaacctcggggaaacggtggagctcaagtgtc |
| | | aagtcctcctgtcgaacccgaccagcggatgcagctggctgttccagccgagaggag |
| | | ctgccgcctcacccaccttcctcctgtacttgagccagaacaagccgaaggccgctga |
| | | gggtctggacacccagcgcttctcgggcaaacggctgggagacacttttgtgctgactc |
| | | tctccgacttccggcgggagaacgagggctactacttctgctctgcgctctccaattcaat |
| | | catgtacttctcacacttcgtgccggtgttcctgcctgccaagcccaccactactccggca |
| | | cccagacctccaactcccgctcccaccatcgcgtcccaacccctttcgctgcgccctga |
| | | agcgtgtcggcctgctgctggaggagccgtgcatacccgcggtctggacttcgcgtgc |
| | | gacatctacatttgggcccctttggctggcacctgtggagtgctgctcctgtcccttgtgat |
| | | caccctgtactgcaaccaccggaataggcggagagtctgcaagtgtccgcggcctgt |
| | | cgtgaagtcaggagataagccgagcctgtccgcacgctacgtgcgggccaagagat |
| | | ctggcagcggcgagggcagaggcagcctgctgacctgcggcgacgtggaggaga |
| | | accccggccccatggactcttggaccttctgctgcgtgagcctgtgcatcctggtggcca |
| | | agcacacagacgccggcgtgatccagtcccctaggcacgaggtgaccgagatggg |
| | | ccaggaggtgacactgcgctgtaagccaatctctggccacaacagcctgttttggtata |
| | | gggagaccatgatgcgcggcctggagctgctgatctacttcaataacaatgtgcccatc |
| | | gacgattccggcatgcctgaggatcggttttctgccaagatgcccaatgccagcttctcc |
| | | acactgaagatccagcctagcgagccaagagactccgccgtgtatttttgcgcctctag |
| | | cccaggcagcaccgatacacagtacttcggaccaggaaccaggctgacagtgctgg |
| | | aggacctgaagaacgtgttcccccctgaggtggccgtgtttgagccctctgaggccga |
| | | gatcagccacacccagaaggccaccctggtgtgcctggcaaccggcttctatcctgat |
| | | cacgtggagctgtcctggtgggtgaacggcaaggaggtgcacagcggcgtgtccac |
| | | agacccacagcccctgaaggagcagccagccctgaatgatagccggtattgcctgtc |
| | | ctctcggctgagagtgtccgccaccttttggcagaacccccggaatcacttcagatgtca |
| | | ggtgcagttttacggcctgtccgagaacgatgagtggacccaggaccgggccaagcc |
| | | tgtgacacagatcgtgtctgccgaggcatggggaagagcagactgtggcttcacctct |
| | | gagagctaccagcagggcgtgctgagcgccaccatcctgtatgagatcctgctgggc |
| | | aaggccacactgtacgccgtcctggtctccgctctggtgctgatggcaatggtcaaaag |
| | | aaaagatagtcggggacgggccaagagatctggcagcggccagtgcaccaactac |
| | | gccctgctgaagctggccggcgacgtggagagcaaccccggccccatggagaaga |
| | | atcccctggctgcccccctgctgatcctgtggtttcacctggactgcgtgtcctctatcctg |
| | | aatgtggaacagagcccacagagcctgcacgtgcaggagggcgactccaccaactt |
| | | cacatgctcttttcctagctccaacttctacgccctgcactggtacagaaaggagaccgc |
| | | aaagtccccagaggccctgttcgtgatgacactgaacggcgatgagaagaagaagg |
| | | gccgcatcagcgccaccctgaatacaaaggagggctactcctatctgtacatcaagg |
| | | gctcccagcctgaggactctgccacctatctgtgcgccctgtacaacaataacgatatg |
| | | cggtttggcgccggcaccagactgacagtgaagccaaacatccagaatccagaccc |
| | | cgccgtgtatcagctgcgggacagcaagtctagcgataagagcgtgtgcctgttcacc |
| | | gactttgattctcagacaaacgtgagccagtccaaggacagcgacgtgtacatcaccg |
| | | acaagacagtgctggatatgagaagcatggacttcaagtctaacagcgccgtggcct |
| | | ggtccaataagtctgatttcgcctgcgccaatgcctttaataactccatcatccccgagg |
| | | ataccttctttccttctccagagtcctcttgtgacgtgaagctggtggagaagtctttcgag |
| | | accgatacaaacctgaattttcagaacctgagcgtgatcggcttcaggatcctgctgctg |
| | | aaggtggccggctttaatctgctgatgaccctgaggctgtggagctcctgaaccggtcc |
| | | gcagtctgacgtacgcgtaatcaacctctggattacaaaatttgtgaaagattgactggt |
| | | attcttaactatgttgctccttttacgctatgtggatacgctgctttaatgcctttgtatcatgct |
| | | attgcttcccgtatggctttcattttctcctccttgtataaatcctggttgctgtctctttatgagg |
| | | agttgtggcccgttgtcaggcaacgtggcgtggtgtgcactgtgtttgctgacgcaaccc |
| | | ccactggttggggcattgccaccacctgtcagctcctttccgggactttcgctttccccctc |
| | | cctattgccacggcggaactcatcgccgcctgccttgcccgctgctggacaggggctc |
| | | ggctgttgggcactgacaattccgtggtgttgtcggggaagctgacgtcctttccatggct |
| | | gctcgcctgtgttgccacctggattctgcgcgggacgtccttctgctacgtcccttcggcc |
| | | ctcaatccagcggaccttccttcccgcggcctgctgccggctctgcggcctcttccgcgt |
| | | cttcgccttcgccctcagacgagtcggatctccctttgggccgcctccccgcc |
| 95 | R11KE WPRE | tgaaagaccccacctgtaggtttggcaagctagcttaagtaacgccattttgcaaggca |
| | | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatggactctt |
| | | ggaccttctgctgcgtgagcctgtgcatcctggtggccaagcacacagacgccggcgt |
| | | gatccagtcccctaggcacgaggtgaccgagatgggccaggaggtgacactgcgct |
| | | gtaagccaatctctggccacaacagcctgttttggtatagggagaccatgatgcgcgg |
| | | cctggagctgctgatctacttcaataacaatgtgcccatcgacgattccggcatgcctga |
| | | ggatcggttttctgccaagatgcccaatgccagcttctccacactgaagatccagccta |
| | | gcgagccaagagactccgccgtgtatttttgcgcctctagcccaggcagcaccgatac |
| | | acagtacttcggaccaggaaccaggctgacagtgctggaggacctgaagaacgtgtt |
| | | cccccctgaggtggccgtgtttgagccctctgaggccgagatcagccacacccagaa |
| | | ggccaccctggtgtgcctggcaaccggcttctatcctgatcacgtggagctgtcctggtg |
| | | ggtgaacggcaaggaggtgcacagcggcgtgtccacagacccacagcccctgaag |
| | | gagcagccagccctgaatgatagccggtattgcctgtcctctcggctgagagtgtccgc |
| | | caccttttggcagaacccccggaatcacttcagatgtcaggtgcagttttacggcctgtc |
| | | cgagaacgatgagtggacccaggaccgggccaagcctgtgacacagatcgtgtctg |
| | | ccgaggcatggggaagagcagactgtggcttcacctctgagagctaccagcagggc |
| | | gtgctgagcgccaccatcctgtatgagatcctgctgggcaaggccacactgtacgccg |
| | | tcctggtctccgctctggtgctgatggcaatggtcaaaagaaaagatagtcggggacg |
| | | ggccaagagatctggcagcggcgccaccaatttcagcctgctgaaacaggccggcg |
| | | acgtggaagagaaccctggccccatggagaagaatcccctggctgcccccctgctg |
| | | atcctgtggtttcacctggactgcgtgtcctctatcctgaatgtggaacagagcccacag |
| | | agcctgcacgtgcaggagggcgactccaccaacttcacatgctcttttcctagctccaa |
| | | cttctacgccctgcactggtacagaaaggagaccgcaaagtccccagaggccctgtt |
| | | cgtgatgacactgaacggcgatgagaagaagaagggccgcatcagcgccaccctg |
| | | aatacaaaggagggctactcctatctgtacatcaagggctcccagcctgaggactctg |
| | | ccacctatctgtgcgccctgtacaacaataacgatatgcggtttggcgccggcaccag |
| | | actgacagtgaagccaaacatccagaatccagaccccgccgtgtatcagctgcggg |
| | | acagcaagtctagcgataagagcgtgtgcctgttcaccgactttgattctcagacaaac |
| | | gtgagccagtccaaggacagcgacgtgtacatcaccgacaagacagtgctggatat |
| | | gagaagcatggacttcaagtctaacagcgccgtggcctggtccaataagtctgatttcg |
| | | cctgcgccaatgcctttaataactccatcatccccgaggataccttctttccttctccagag |
| | | tcctcttgtgacgtgaagctggtggagaagtctttcgagaccgatacaaacctgaattttc |
| | | agaacctgagcgtgatcggcttcaggatcctgctgctgaaggtggccggctttaatctg |
| | | ctgatgaccctgaggctgtggagctcctgaaccggtccgcagtctgacgtacgcgtaat |
| | | caacctctggattacaaaatttgtgaaagattgactggtattcttaactatgttgctcctttta |
| | | cgctatgtggatacgctgctttaatgcctttgtatcatgctattgcttcccgtatggctttcatttt |
| | | ctcctccttgtataaatcctggttgctgtctctttatgaggagttgtggcccgttgtcaggca |
| | | acgtggcgtggtgtgcactgtgtttgctgacgcaacccccactggttggggcattgccac |
| | | cacctgtcagctcctttccgggactttcgctttccccctccctattgccacggcggaactca |
| | | tcgccgcctgccttgcccgctgctggacaggggctcggctgttgggcactgacaattcc |
| | | gtggtgttgtcggggaagctgacgtcctttccatggctgctcgcctgtgttgccacctggat |
| | | tctgcgcgggacgtccttctgctacgtcccttcggccctcaatccagcggaccttccttcc |
| | | cgcggcctgctgccggctctgcggcctcttccgcgtcttcgccttcgccctcagacgagt |
| | | cggatctccctttgggccgcctccccgcc |
| 96 | CD8 WPRE | tgaaagaccccacctgtaggtttggcaagctagcttaagtaacgccattttgcaaggca |
| | | tggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaacc |
| | | atcagatgtttccagggtgccccaaggacctgaaaatgaccctgtgccttatttgaacta |
| | | accaatcagttcgcttctcgcttctgttcgcgcgcttctgctccccgagctcaataaaaga |
| | | gcccacaacccctcactcagcggccgccccgggtcgacgctaccaccatgcgcccg |
| | | agactgtggcttctgctcgccgcgcaactgactgtcctgcacggaaacagcgtgctgc |
| | | agcagacaccggcctacatcaaagtgcagaccaacaagatggtcatgctgtcctgcg |
| | | aggccaagatttccctctccaacatgcggatctattggttgcggcagagacaggcgcct |
| | | tcctcggactcccaccatgagttcttggccctgtgggactccgccaagggaactattcac |
| | | ggcgaagaagtggaacaggagaagatcgccgtgtttcgcgatgcctcccgctttatac |
| | | tgaatctgacctccgtgaagcccgaagatagcgggatctacttttgcatgattgtgggct |
| | | cacccgaactgaccttcgggaagggcactcagctgagcgtggtggacttcctccccac |
| | | taccgcccaacccactaagaagtcaaccctgaagaagcgggtttgcagactcccac |
| | | ggccggaaacgcagaagggtccgctgtgttccccgatcaccctggggctccttgtggc |
| | | tggagtgctggtccttctggtgtcccttggcgtcgccattcacctctgctgccggagaagg |
| | | agggccagactgaggttcatgaagcagcctcagggagaggggatcagtggcactttc |
| | | gtgccacaatgcctccatggctactattccaacaccaccacctcgcaaaagctgctga |
| | | acccctggatcctgaaaacccgggccaagagatctggcagcggcgccaccaatttc |
| | | agcctgctgaaacaggccggcgacgtggaagagaaccctggccccatggcgcttcc |
| | | cgtgaccgcactcctgttgccccttgccctgctgttgcacgccgcacgaccttcccaattc |
| | | cgggtgtcccctctggatcgcacctggaacctcggggaaacggtggagctcaagtgtc |
| | | aagtcctcctgtcgaacccgaccagcggatgcagctggctgttccagccgagaggag |
| | | ctgccgcctcacccaccttcctcctgtacttgagccagaacaagccgaaggccgctga |
| | | gggtctggacacccagcgcttctcgggcaaacggctgggagacacttttgtgctgactc |
| | | tctccgacttccggcgggagaacgagggctactacttctgctctgcgctctccaattcaat |
| | | catgtacttctcacacttcgtgccggtgttcctgcctgccaagcccaccactactccggca |
| | | cccagacctccaactcccgctcccaccatcgcgtcccaacccctttcgctgcgccctga |
| | | agcgtgtcggcctgctgctggaggagccgtgcatacccgcggtctggacttcgcgtgc |
| | | gacatctacatttgggcccctttggctggcacctgtggagtgctgctcctgtcccttgtgat |
| | | caccctgtactgcaaccaccggaataggcggagagtctgcaagtgtccgcggcctgt |
| | | cgtgaagtcaggagataagccgagcctgtccgcacgctacgtgtgaaccggtccgca |
| | | gtctgacgtacgcgtaatcaacctctggattacaaaatttgtgaaagattgactggtattct |
| | | taactatgttgctccttttacgctatgtggatacgctgctttaatgcctttgtatcatgctattgc |
| | | ttcccgtatggctttcattttctcctccttgtataaatcctggttgctgtctctttatgaggagttgt |
| | | ggcccgttgtcaggcaacgtggcgtggtgtgcactgtgtttgctgacgcaacccccact |
| | | ggttggggcattgccaccacctgtcagctcctttccgggactttcgctttccccctccctatt |
| | | gccacggcggaactcatcgccgcctgccttgcccgctgctggacaggggctcggctgt |
| | | tgggcactgacaattccgtggtgttgtcggggaagctgacgtcctttccatggctgctcgc |
| | | ctgtgttgccacctggattctgcgcgggacgtccttctgctacgtcccttcggccctcaatc |
| | | cagcggaccttccttcccgcggcctgctgccggctctgcggcctcttccgcgtcttcgcct |
| | | tcgccctcagacgagtcggatctccctttgggccgcctccccgcc |
| 97 | RD114TR | MKLPTGMVILCSLIIVRAGFDDPRKAIALVQKQHGKPCECSGG |
| | | QVSEAPPNSIQQVTCPGKTAYLMTNQKWKCRVTPKISPSGG |
| | | ELQNCPCNTFQDSMHSSCYTEYRQCRRINKTYYTATLLKIRS |
| | | GSLNEVQILQNPNQLLQSPCRGSINQPVCWSATAPIHISDGG |
| | | GPLDTKRVWTVQKRLEQIHKAMTPELQYHPLALPKVRDDLSL |
| | | DARTFDILNTTFRLLQMSNFSLAQDCWLCLKLGTPTPLAIPTP |
| | | SLTYSLADSLANASCQIIPPLLVQPMQFSNSSCLSSPFINDTEQ |
| | | IDLGAVTFTNCTSVANVSSPLCALNGSVFLCGNNMAYTYLPQ |
| | | NWTRLCVQASLLPDIDINPGDEPVPIPAIDHYIHRPKRAVQFIP |
| | | LLAGLGITAAFTTGATGLGVSVTQYTKLSHQLISDVQVLSGTIQ |
| | | DLQDQVDSLAEVVLQNRRGLDLLTAEQGGICLALQEKCCFYA |
| | | NKSGIVRNKIRTLQEELQKRRESLASNPLWTGLQGFLPYLLPL |
| | | LGPLLTLLLILTIGPCVFNRLVQFVKDRISVVQALVLTQQYHQL |
| | | KPL |
| 265 | WPREmut1 | cagtctgacgtacgcgtaatcaacctctggattacaaaatttgtgaaagattgactggtat |
| | | tcttaactatgttgctccttttacgctatgtggatacgctgctttaatgcctttgtatcatgctatt |
| | | gcttcccgtatggctttcattttctcctccttgtataaatcctggttgctgtctctttatgaggagt |
| | | tgtggcccgttgtcaggcaacgtggcgtggtgtgcactgtgtttgctgacgcaaccccca |
| | | ctggttggggcattgccaccacctgtcagctcctttccgggactttcgctttccccctcccta |
| | | ttgccacggcggaactcatcgccgcctgccttgcccgctgctggacaggggctcggct |
| | | gttgggcactgacaattccgtggtgttgtcggggaaatcatcgtcctttccttggctgctcg |
| | | cctgtgttgccacctggattctgcgcgggacgtccttctgctacgtcccttcggccctcaat |
| | | ccagcggaccttccttcccgcggcctgctgccggctctgcggcctcttccgcgtcttcgc |
| | | cttcgccctcagacgagtcggatctccctttgggccgcctccccgcc |
| 266 | WPREmut2 | gagcatcttaccgccatttatacccatatttgttctgtttttcttgatttgggtatacatttaaatg |
| | | ttaataaaacaaaatggtggggcaatcatttacattttttgggatatgtaattactagttcag |
| | | gtgtattgccacaagacaaacttgttaagaaactttcccgttatttacgctctgttcctgtta |
| | | atcaacctctggattacaaaatttgtgaaagattgactgatattcttaactttgttgctcctttt |
| | | acgctgtgtggatttgctgctttattgcctctgtatcttgctattgcttcccgtacggctttcgtttt |
| | | ctcctccttgtataaatcctggttgctgtctctttttgaggagttgtggcccgttgtccgtcaac |
| | | gtggcgtggtgtgctctgtgtttgctgacgcaacccccactggctggggcattgccacca |
| | | cctgtcaactcctttctgggactttcgctttccccctcccgatcgccacggcagaactcatc |
| | | gccgcctgccttgcccgctgctggacaggggctaggttgctgggcactgataattccgt |
| | | ggtgttgtc |

TABLE 3
TAA Peptide sequences

| SEQ | Amino Acid |
|---|---|
| ID NO: | Sequence |
| 98 | YLYDSETKNA |
| 99 | HLMDQPLSV |
| 100 | GLLKKINSV |
| 101 | FLVDGSSAL |
| 102 | FLFDGSANLV |
| 103 | FLYKIIDEL |
| 104 | FILDSAETTTL |
| 105 | SVDVSPPKV |
| 106 | VADKIHSV |
| 107 | IVDDLTINL |
| 108 | GLLEELVTV |
| 109 | TLDGAAVNQV |
| 110 | SVLEKEIYSI |
| 111 | LLDPKTIFL |
| 112 | YTFSGDVQL |
| 113 | YLMDDFSSL |
| 114 | KVWSDVTPL |
| 115 | LLWGHPRVALA |
| 116 | KIWEELSVLEV |
| 117 | LLIPFTIFM |
| 118 | FLIENLLAA |
| 119 | LLWGHPRVALA |
| 120 | FLLEREQLL |
| 121 | SLAETIFIV |
| 122 | TLLEGISRA |
| 123 | KIQEILTQV |
| 124 | VIFEGEPMYL |
| 125 | SLFESLEYL |
| 126 | SLLNQPKAV |
| 127 | GLAEFQENV |
| 128 | KLLAVIHEL |
| 129 | TLHDQVHLL |
| 130 | TLYNPERTITV |
| 131 | KLQEKIQEL |
| 132 | SVLEKEIYSI |
| 133 | RVIDDSLVVGV |
| 134 | VLFGELPAL |
| 135 | GLVDIMVHL |
| 136 | FLNAIETAL |
| 137 | ALLQALMEL |
| 138 | ALSSSQAEV |
| 139 | SLITGQDLLSV |
| 140 | QLIEKNWLL |
| 141 | LLDPKTIFL |
| 142 | RLHDENILL |
| 143 | YTFSGDVQL |
| 144 | GLPSATTTV |
| 145 | GLLPSAESIKL |
| 146 | KTASINQNV |
| 147 | SLLQHLIGL |
| 148 | YLMDDFSSL |
| 149 | LMYPYIYHV |
| 150 | KVWSDVTPL |
| 151 | LLWGHPRVALA |
| 152 | VLDGKVAVV |
| 153 | GLLGKVTSV |
| 154 | KMISAIPTL |
| 155 | GLLETTGLLAT |
| 156 | TLNTLDINL |
| 157 | VIIKGLEEI |
| 158 | YLEDGFAYV |
| 159 | KIWEELSVLEV |
| 160 | LLIPFTIFM |
| 161 | ISLDEVAVSL |
| 162 | KISDFGLATV |
| 163 | KLIGNIHGNEV |
| 164 | ILLSVLHQL |
| 165 | LDSEALLTL |
| 166 | VLQENSSDYQSNL |
| 167 | HLLGEGAFAQV |
| 168 | SLVENIHVL |
| 169 | YTFSGDVQL |
| 170 | SLSEKSPEV |
| 171 | AMFPDTIPRV |
| 172 | FLIENLLAA |
| 173 | FTAEFLEKV |
| 174 | ALYGNVQQV |
| 175 | LFQSRIAGV |
| 176 | ILAEEPIYIRV |
| 177 | FLLEREQLL |
| 178 | LLLPLELSLA |
| 179 | SLAETIFIV |
| 180 | AILNVDEKNQV |
| 181 | RLFEEVLGV |
| 182 | YLDEVAFML |
| 183 | KLIDEDEPLFL |
| 184 | KLFEKSTGL |
| 185 | SLLEVNEASSV |
| 186 | GVYDGREHTV |
| 187 | GLYPVTLVGV |
| 188 | ALLSSVAEA |
| 189 | TLLEGISRA |
| 190 | SLIEESEEL |
| 191 | ALYVQAPTV |
| 192 | KLIYKDLVSV |
| 193 | ILQDGQFLV |
| 194 | SLLDYEVSI |
| 195 | LLGDSSFFL |
| 196 | VIFEGEPMYL |
| 197 | ALSYILPYL |
| 198 | FLFVDPELV |
| 199 | SEWGSPHAAVP |
| 200 | ALSELERVL |
| 201 | SLFESLEYL |
| 202 | KVLEYVIKV |
| 203 | VLLNEILEQV |
| 204 | SLLNQPKAV |
| 205 | KMSELQTYV |
| 206 | ALLEQTGDMSL |
| 207 | VIIKGLEEITV |
| 208 | KQFEGTVEI |
| 209 | KLQEEIPVL |
| 210 | GLAEFQENV |
| 211 | NVAEIVIHI |
| 212 | ALAGIVTNV |
| 213 | NLLIDDKGTIKL |
| 214 | VLMQDSRLYL |
| 215 | KVLEHVVRV |
| 216 | LLWGNLPEI |
| 217 | SLMEKNQSL |
| 218 | KLLAVIHEL |
| 219 | ALGDKFLLRV |
| 220 | FLMKNSDLYGA |
| 221 | KLIDHQGLYL |
| 222 | GPGIFPPPPPQP |
| 223 | ALNESLVEC |
| 224 | GLAALAVHL |
| 225 | LLLEAVWHL |
| 226 | SIIEYLPTL |
| 227 | TLHDQVHLL |
| 228 | SLLMWITQC |
| 229 | FLLDKPQDLSI |
| 230 | YLLDMPLWYL |
| 231 | GLLDCPIFL |
| 232 | VLIEYNFSI |
| 233 | TLYNPERTITV |
| 234 | AVPPPPSSV |
| 235 | KLQEELNKV |
| 236 | KLMDPGSLPPL |
| 237 | ALIVSLPYL |
| 238 | FLLDGSANV |
| 239 | ALDPSGNQLI |
| 240 | ILIKHLVKV |
| 241 | VLLDTILQL |
| 242 | HLIAEIHTA |
| 243 | SMNGGVFAV |
| 244 | MLAEKLLQA |
| 245 | YMLDIFHEV |
| 246 | ALWLPTDSATV |
| 247 | GLASRILDA |
| 248 | ALSVLRLAL |
| 249 | SYVKVLHHL |
| 250 | VYLPKIPSW |
| 251 | NYEDHFPLL |
| 252 | VYIAELEKI |
| 253 | VHFEDTGKTLLF |
| 254 | VLSPFILTL |
| 255 | HLLEGSVGV |

### Example 2

### γδ T Cell Manufacturing

To isolate γδ T cells, in an aspect, γδ T cells may be isolated from a subject or from a complex sample of a subject. In an aspect, a complex sample may be a peripheral blood sample, a cord blood sample, a tumor, a stem cell precursor, a tumor biopsy, a tissue, a lymph, or from epithelial sites of a subject directly contacting the external milieu or derived from stem precursor cells. γδ T cells may be directly isolated from a complex sample of a subject, for example, by sorting γδ T cells that express one or more cell surface markers with flow cytometry techniques. Wild-type γδ T cells may exhibit numerous antigen recognition, antigen-presentation, co-stimulation, and adhesion molecules that can be associated with a γδ T cells. One or more cell surface markers, such as specific γδ TCRs, antigen recognition, antigen-presentation, ligands, adhesion molecules, or co-stimulatory molecules may be used to isolate wild-type γδ T cells from a complex sample. Various molecules associated with or expressed by γδ T-cells may be used to isolate γδ T cells from a complex sample, e.g., isolation of mixed population of Vδ1+, Vδ2+, Vδ3+ cells or any combination thereof.

For example, peripheral blood mononuclear cells can be collected from a subject, for example, with an apheresis machine, including the Ficoll-Paque™ PLUS (GE Healthcare) system, or another suitable device/system. γδ T-cell(s), or a desired subpopulation of γδ T-cell(s), can be purified from the collected sample with, for example, with flow cytometry techniques. Cord blood cells can also be obtained from cord blood during the birth of a subject.

Positive and/or negative selection of cell surface markers expressed on the collected γδ T cells can be used to directly isolate γδ T cells, or a population of γδ T cells expressing similar cell surface markers from a peripheral blood sample, a cord blood sample, a tumor, a tumor biopsy, a tissue, a lymph, or from an epithelial sample of a subject. For instance, γδ T cells can be isolated from a complex sample based on positive or negative expression of CD2, CD3, CD4, CD8, CD24, CD25, CD44, Kit, TCR α, TCR β, TCR α, TCR δ, NKG2D, CD70, CD27, CD30, CD16, CD337 (NKp30), CD336 (NKp46), OX40, CD46, CCR7, and other suitable cell surface markers.

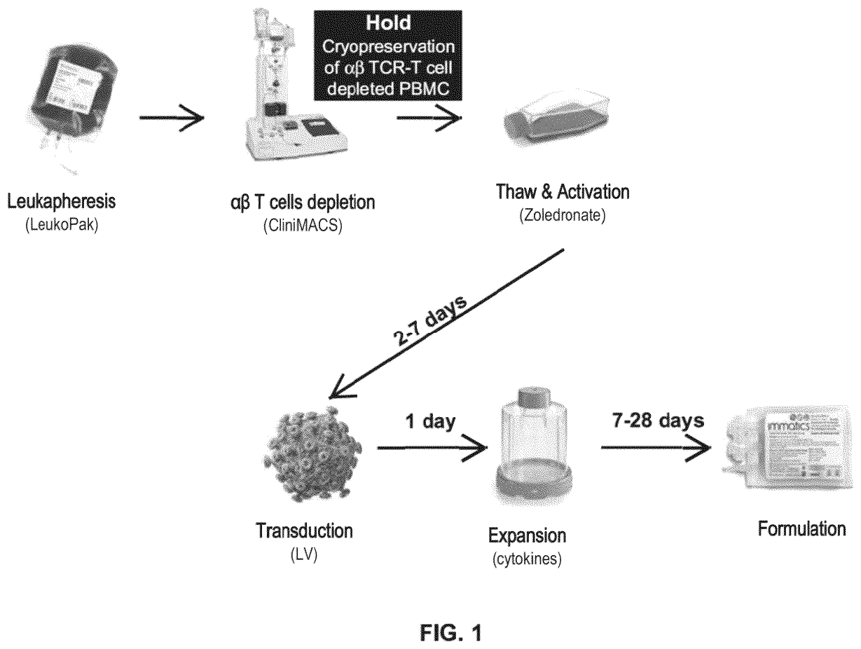

FIG. 1 shows γδ T cell manufacturing according to an embodiment of the present disclosure. This process may include collecting or obtaining white blood cells or PBMC from leukapheresis products. Leukapheresis may include collecting whole blood from a donor and separating the components using an apheresis machine. An apheresis machine separates out desired blood components and returns the rest to the donor's circulation. For instance, white blood cells, plasma, and platelets can be collected using apheresis equipment, and the red blood cells and neutrophils are returned to the donor's circulation. Commercially available leukapheresis products may be used in this process. Another way to obtain white blood cells is to obtain them from the buffy coat. To isolate the buffy coat, whole anticoagulated blood is obtained from a donor and centrifuged. After centrifugation, the blood is separated into plasma, red blood cells, and buffy coat. The buffy coat is the layer located between the plasma and red blood cell layers. Leukapheresis collections may result in higher purity and considerably increased mononuclear cell content than that achieved by buffy coat collection. The mononuclear cell content possible with leukapheresis may typically be 20 times higher than that obtained from the buffy coat. In order to enrich for mononuclear cells, the use of a Ficoll gradient may be needed for further separation.

To deplete αβ T cells from PBMC, αβ TCR-expressing cells may be separated from the PBMC by magnetic separation, e.g., using CliniMACS® magnetic beads coated with anti-αβ TCR antibodies, followed by cryopreserving αβ TCR-T cells depleted PBMC. To manufacture “off-the-shelf” T-cell products, cryopreserved αβ TCR-T cells depleted PBMC may be thawed and activated in small/mid-scale, e.g., 24 to 4-6 well plates or T75/T175 flasks, or in large scale, e.g., 50 ml-100 liter bags, in the presence of aminobisphosphonate, e.g., zoledronate, and/or isopentenylpyrophosphate (IPP) and/or cytokines, e.g., interleukin 2 (IL-2), interleukin 15 (IL-15), and/or interleukin 18 (IL-18), and/or other activators, e.g., Toll-like receptor 2 (TLR2) ligand, for 1-10 days, e.g., 2-7 days.

FIG. 1 shows the activated T cells may be engineered by transducing with a viral vector, such as lentiviral vector, expressing exogenous genes of interest, such as αβ TCRs against specific cancer antigen and CD8, into isolated γδ T cells. Transduction may be carried out once or multiple times to achieve stable transgene expression in small scale, e.g., 24 to 4-6 well plates, or mid/large scale for ½-5 days, e.g., 1 day.

FIG. 1 further shows expansion of the transduced or engineered γδ T cells may be carried out in the presence of cytokines, e.g., IL-2, IL-15, IL-18, and others, in small/mid-scale, e.g., flasks/G-Rex, or in large scale, e.g., 50 ml-100-liter bags, for 7-35 days, e.g., 7-28 days. The expanded transduced T cell products may then be cryopreserved as “off-the-shelf” T-cell products for infusion into patients.

### Example 3

### Lentiviral Viral Vectors

The lentiviral vectors used herein contain several elements previously shown to enhance vector function, including a central polypurine tract (cPPT) for improved replication and nuclear import, a promoter from the murine stem cell virus (MSCV) (SEQ ID NO: 1), which has been shown to lessen vector silencing in some cell types, a woodchuck hepatitis virus posttranscriptional responsive element (WPRE) (SEQ ID NO: 9) for improved transcriptional termination, and the backbone was a deleted 3′-LTR self-inactivating (SIN) vector design that may have improved safety, sustained gene expression and anti-silencing properties (Yang et al. Gene Therapy (2008) 15, 1411-1423, the content of which is incorporated by reference in its entirety).

In an aspect, vectors, constructs, or sequences described herein comprise mutated forms of WPRE. In another aspect, sequences or vectors described herein comprise mutations in WPRE version 1, e.g., WPREmut1 (SEQ ID NO: 265), or WPRE version 2, e.g., WPREmut2 (SEQ ID NO: 266). In an aspect, WPRE mutants comprise at most one mutation, at most two mutations, at most three mutations, at least four mutations, or at most five mutations. In an aspect, vectors, constructs, or sequences described herein do not comprise WPRE. In another aspect, the disclosure provides for one, two, three, four, five, ten, or 20 substitutions in one of SEQ ID NO: 91-96.

In another aspect, vectors, constructs, or sequences described herein do not include an X protein promoter.

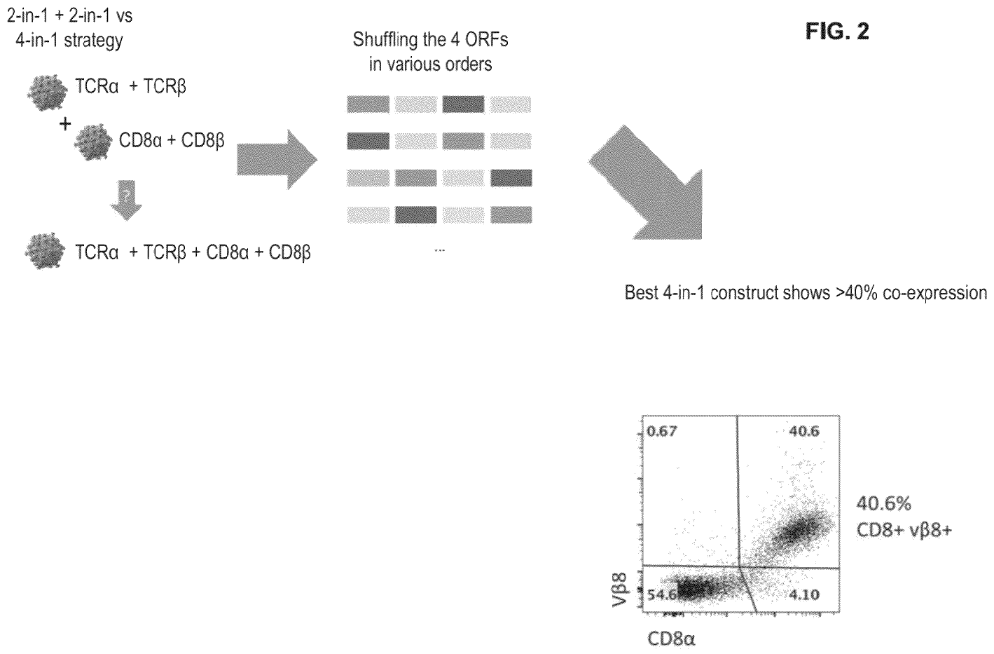

To obtain optimal co-expression levels of TCRαβ and CD8αβ in the transduced γδ T cells, lentiviral vectors with various designs were generated. FIG. 2 shows T cells may be transduced with two separate lentiviral vectors (2-in-1) expressing TCRαβ or CD8αβ and a single lentiviral vector (4-in-1) co-expressing TCRαβ and CD8αβ. In the 4-in-1 vector, the nucleotides encoding TCRα chain, TCRβ chain, CD8α chain, and CD8β chain may be shuffled in various orders. Various 4-in-1 vectors, thus generated, may be used to transduce γδ T cells, followed by measuring TCR/CD8 co-expression levels of the transduced cells using techniques known in the art, e.g., flow cytometry.

To generate lentiviral vectors co-expressing TCRαβ and CD8αβ, a nucleotide encoding furin-linker (GSG or SGSG (SEQ ID NO: 8))-2A peptide may be positioned between TCRα chain and TCRβ chain, between CD8α chain and CD8β chain, and between a TCR chain and a CD8 chain to enable highly efficient gene expression. The 2A peptide may be selected from P2A (SEQ ID NO: 3), T2A (SEQ ID NO: 4), E2A (SEQ ID NO: 5), or F2A (SEQ ID NO: 6).

Lentiviral viral vectors may also contain post-transcriptional regulatory element (PRE), such as Woodchuck PRE (WPRE) (SEQ ID NO: 9) to enhance the expression of the transgene by increasing both nuclear and cytoplasmic mRNA levels. One or more regulatory elements including mouse RNA transport element (RTE), the constitutive transport element (CTE) of the simian retrovirus type 1 (SRV-1), and the 5′ untranslated region of the human heat shock protein 70 (Hsp70 5′UTR) may also be used and/or in combination with WPRE to increase transgene expression.

Lentiviral vectors may be pseudotyped with RD114TR (SEQ ID NO: 97), which is a chimeric glycoprotein containing an extracellular and transmembrane domain of feline endogenous virus (RD114) fused to cytoplasmic tail (TR) of murine leukemia virus. Other viral envelop proteins, such as VSV-G env, MLV 4070A env, RD114 env, chimeric envelope protein RD114pro, baculovirus GP64 env, or GALV env, or derivatives thereof, may also be used.

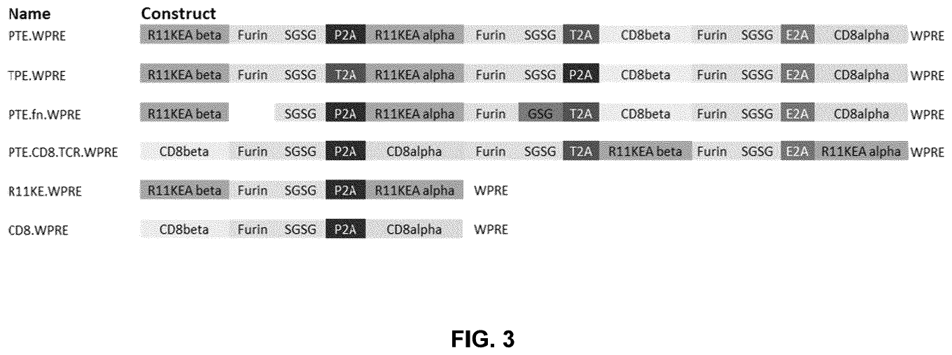

FIG. 3 shows four different 4-in-1 vectors, i.e., PTE WPRE (SEQ ID NO: 91), TPE WPRE (SEQ ID NO: 92), PTE fn WPRE (SEQ ID NO: 93), and PTE CD8 TCR WPRE (SEQ ID NO: 94), co-expressing TCRαβ (R11KEA) and CD8αβ, and two 2-in-1 vectors, i.e., R11KE WPRE (SEQ ID NO: 95), expressing TCRαβ (R11KEA) and CD8 WPRE (SEQ ID NO: 96) expressing CD8αβ. TCRαβ (R11KEA) binds to PRAME-004 (SLLQHLIGL) (SEQ ID NO: 147) in a complex with an MHC molecule.

### Example 4

### Co-Expression of TCR and CD8

γδ T cells obtained from Donor 1 and Donor 2 were manufactured by the process shown in FIG. 1. On Day 3 or Day 6 post-activation with zoledronate, IL2, and IL15, γδ T cells were transduced with lentivirus, pseudotyped with RD114TR, e.g., PTE WPRE (SEQ ID NO: 91), TPE WPRE (SEQ ID NO: 92), PTE fn WPRE (SEQ ID NO: 93), and PTE CD8 TCR WPRE (SEQ ID NO: 94), followed by measuring co-expression levels of R11KEA and CD8 using flow cytometry. Transduction efficiency was assessed using antibodies specific to TCR (Vβ8) and CD8 (CD8α) via flow cytometry.

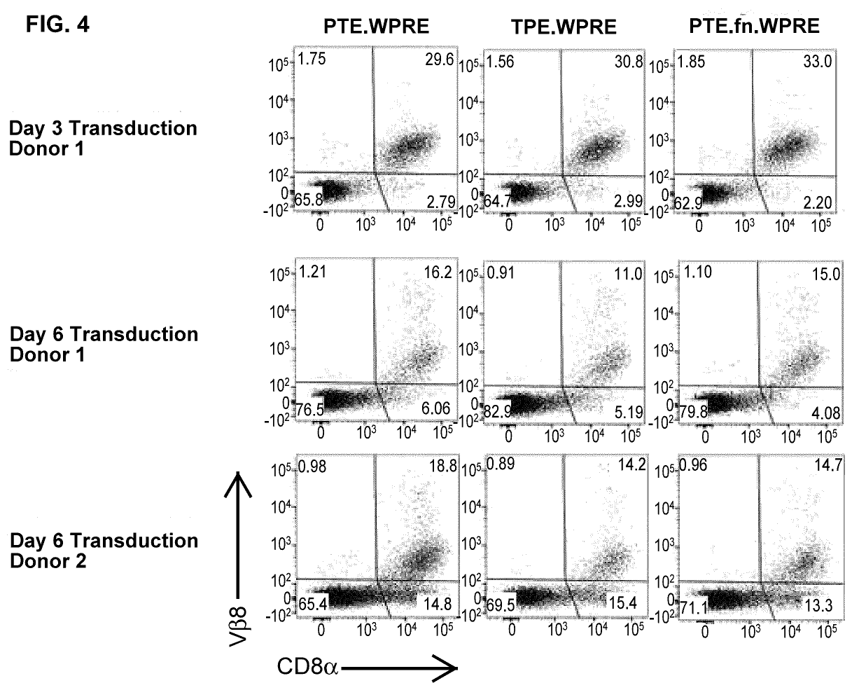

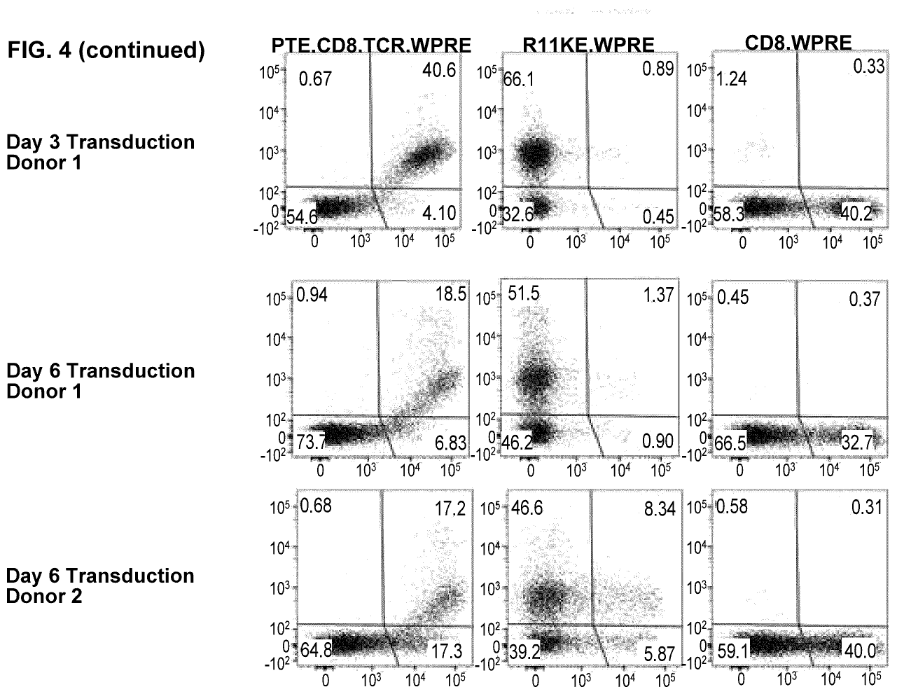

FIG. 4 shows, in γδ T cells from Donor 1, co-expression levels of R11KEA and CD8 resulted from transduction with PTE CD8 TCR WPRE, i.e., 40.5% (Day 3) and 18.5% (Day 6), are higher than that from transduction with PTE WPRE (29.6% (Day 3), 16.2% (Day 6)), TPE WPRE (30.8% (Day 3), 11.0% (Day 6)), and PTE fn WPRE (33.0% (Day 3), 15.0% (Day 6)). In γδ T cells from Donor 2, co-expression levels of R11KEA and CD8 on Day 6 post-activation resulted from transduction with PTE WPRE, i.e., 18.8%, is higher than that from transduction with TPE WPRE (14.2%), PTE fn WPRE (14.7%), and PTE CD8 TCR WPRE (17.2%). As controls, background levels of R11KEA and CD8 were detected in γδ T cells transduced separately with 2-in-1 vectors, i.e., TCRαβ (R11KEA) or CD8 WPRE.

### Example 5

### Effects on Transgene Expression and Functionality of 4-In-1 Viral Vectors, e.g., Lentiviral Vectors, Containing Sequences Encoding CD8αβ Chains and Sequences Encoding TCRαβ Chains Located at Different Positions in the Vectors.

WO 2019/204662 describes CD4+ cells that express an exogenous CD8αβ co-receptor and one or more exogenous engineered antigen receptors, e.g., TCRs. Table 4 shows a comparison between the 4-in-1 constructs described in WO 2019/204662 and that of according to aspects of the present disclosure.

TABLE 4

| | WO 2019/204662 | Aspects described herein |
|---|---|---|
| Orientation of transgene | TCRβ-TCRα-CD8α-CD8β | CD8β-CD8α-TCRβ-TCRα |
| (from 5′ end to 3′ end | | |
| direction) | | |
| Sources of CD8αβ | GenBank | codon optimized (for |
| sequences | | enhancing expression) |
| 2A linkers | 2A | 2A + Furin linker (for |
| | | promoting efficient cleavage |
| | | of residual 2A sequences for |
| | | gene of interest) |
| Cell Type | CD4+ cells and CD8+ cells | γδ T cells (low (0-20%) CD8 |
| | | expression and no CD4 |
| | | expression) |
| Virus and Pseudotype | retrovirus and RD114 | lentivirus and RD114TR and |
| | | VSV-G |

The open reading frame (ORF) of the nucleic acid molecules of the present disclosure may be at least partially codon-optimized. Codon-optimization is based on the finding that the translation efficiency may be determined by a different frequency in the occurrence of transfer RNAs (tRNAs) in cells. Thus, the open reading frames of nucleic acid molecules of the present disclosure may be modified compared to the corresponding wild type coding region such that at least one codon of the wild type sequence that codes for a tRNA, which is relatively rare in the cell, may be exchanged for a codon, which codes for a tRNA, which is comparably frequent in the cell and may carry the same amino acid as the relatively rare tRNA. By this modification, the open reading frame of nucleic acid molecules of the present disclosure may be modified such that codons, for which frequently occurring tRNAs are available may replace codons, which correspond to rare tRNAs. In other words, according to the present disclosure, by such a modification all codons of the wild type open reading frame, which code for a rare tRNA, may be exchanged for a codon, which codes for a tRNA, which is more frequent in the cell and which carries the same amino acid as the rare tRNA. Which tRNAs occur relatively frequently in the cell and which, in contrast, occur relatively rarely is known to a person skilled in the art; e.g., Akashi, Curr. Opin. Genet. Dev. 2001, 11 (6): 660-666, the contents of which are incorporated by reference in their entireties. In some embodiments, open reading frames of nucleic acid molecules of the present disclosure may be codon-optimized, preferably with respect to the system, in which the nucleic acid molecules of the present disclosure are to be expressed, preferably with respect to the system, in which the nucleic acid molecules of the present disclosure are to be translated. Preferably, the codon usage of open reading frames of the nucleic acid molecules of the present disclosure may be codon-optimized according to mammalian codon usage, more preferably, according to human codon usage. Preferably, the open reading frame may be codon-optimized and G/C-content modified.

To determine which transgene orientation provide better transgene expression and functionality, three 4-in-1 viral vectors each containing sequences encoding TCRαβ chains located upstream from sequences encoding CD8αβ chains, e.g., PTE.WPRE (SEQ ID NO: 91), TPE.WPRE (SEQ ID NO: 92), and PTE.fn.WPRE (SEQ ID NO: 93), and a 4-in-1 viral vector containing sequences encoding CD8αβ chains located upstream from sequences encoding TCRαβ chains, e.g., PTE.CD8.TCR.WPRE (SEQ ID NO: 94), were transduced into γδ T cells, followed by fluorescence-activated cell sorting (FACS) analysis using fluorescently-tagged anti-CD8 antibodies and fluorescently-tagged anti-TCR Vβ8 (Vb8) antibodies to detect the expression of CD8 and TCR, respectively, on the cell surface.

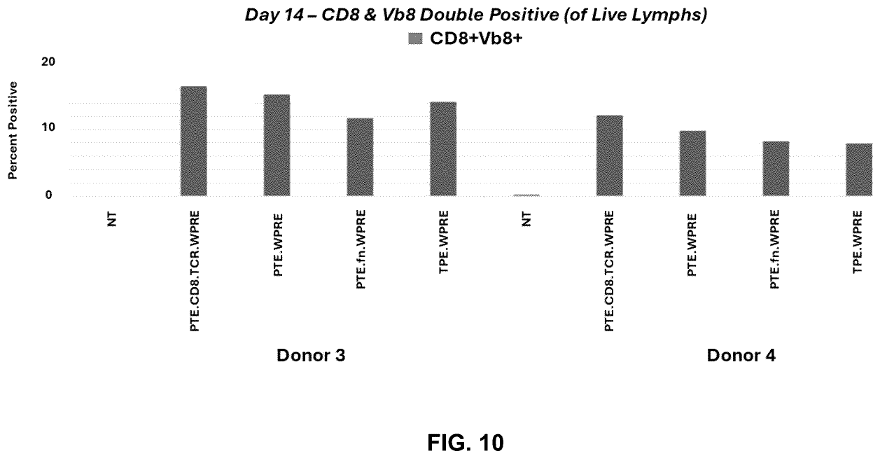

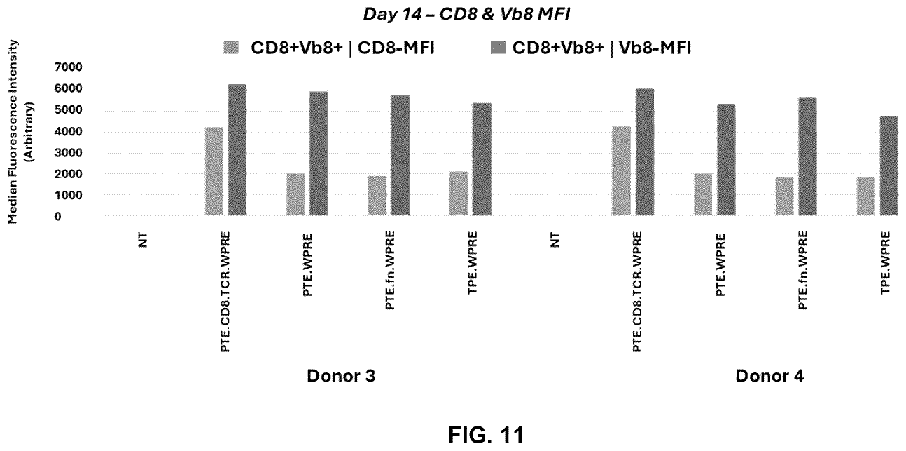

FIGS. 10 and 11 show that γδ T cells obtained from Donor 3 and Donor 4 transduced with the 4-in-1 viral vector containing PTE.CD8.TCR.WPRE results in the highest expression of both CD8 and TCR on the cell surface at Day 14 of manufacturing as compared with that transduced with 4-in-1 viral vector containing PTE.WPRE, TPE.WPRE, or PTE.fn.WPRE, based on the % of CD8+Vb8+ double-positive cells (FIG. 10) and the MFI of CD8 or the MFI of Vb8 (FIG. 11).

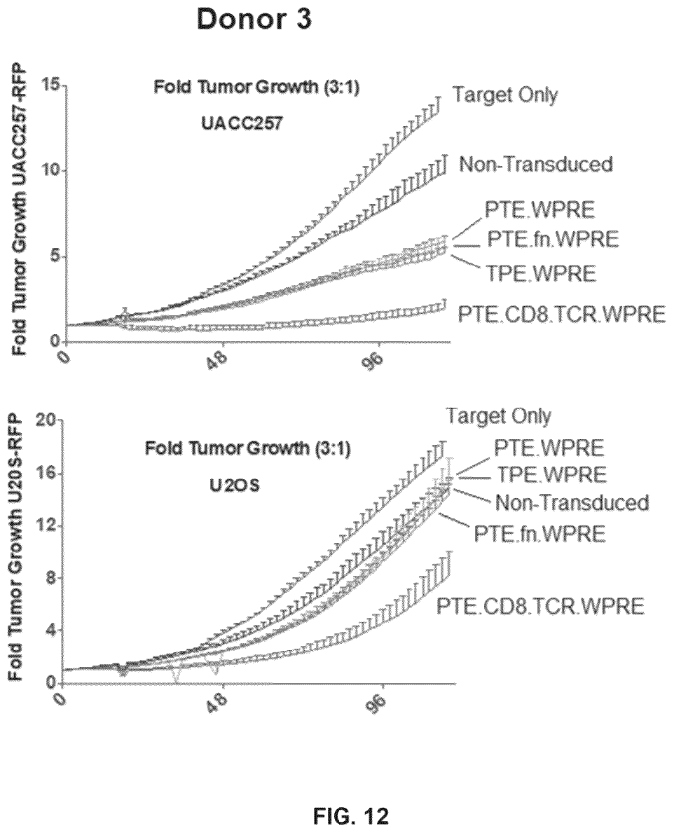

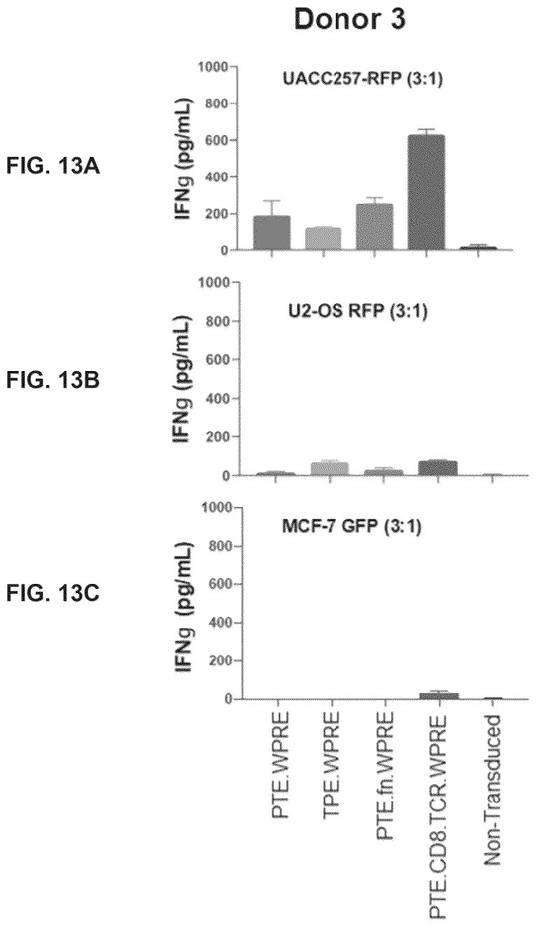

The high expression of both CD8 and TCR on the cell surface of γδ T cells transduced with 4-in-1 viral vector containing PTE.CD8.TCR.WPRE correlates well with their in vitro killing activity. For example, FIG. 12 shows γδ T cells obtained from Donor 3 transduced with 4-in-1 viral vector containing PTE.CD8.TCR.WPRE exhibits the best killing activity against both the high target peptide presenting cell line UACC257 (top panel) and the low target peptide presenting cell line U2OS (bottom panel) as compared with that transduced with 4-in-1 viral vector containing PTE.WPRE, TPE.WPRE, or PTE.fn.WPRE, FIGS. 13A-13C show amount of IFN-γ secretion by the corresponding γδ T cells transduced with 4-in-1 viral vector containing PTE.CD8.TCR.WPRE, PTE.WPRE, TPE.WPRE, or PTE.fn.WPRE in the presence of target cells, e.g., UACC257 (FIG. 13A), U2OS (FIG. 13B), and target-negative cell line MCF-7 (FIG. 13C).

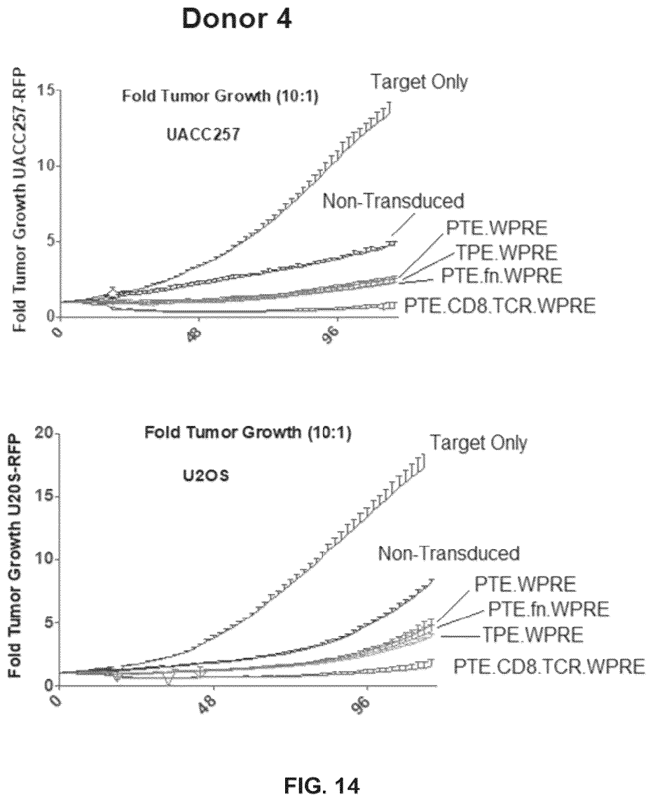

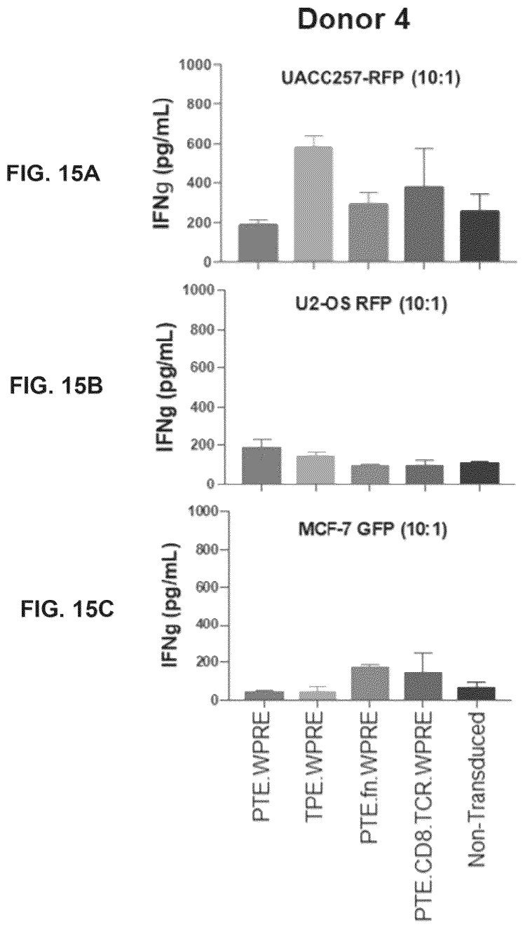

FIG. 14 shows γδ T cells obtained from Donor 4 transduced with 4-in-1 viral vector containing PTE.CD8.TCR.WPRE also exhibits the best killing activity against both UACC257 (top panel) and U2OS (bottom panel) as compared with that transduced with 4-in-1 viral vector containing PTE.WPRE, TPE.WPRE, or PTE.fn.WPRE, FIGS. 15A-15C show amount of IFN-γ secretion by the corresponding γδ T cells transduced with 4-in-1 viral vector containing PTE.CD8.TCR.WPRE, PTE.WPRE, TPE.WPRE, or PTE.fn.WPRE in the presence of target cells, e.g., UACC257 (FIG. 15A), U2OS (FIG. 15B), and MCF-7 (FIG. 15C).

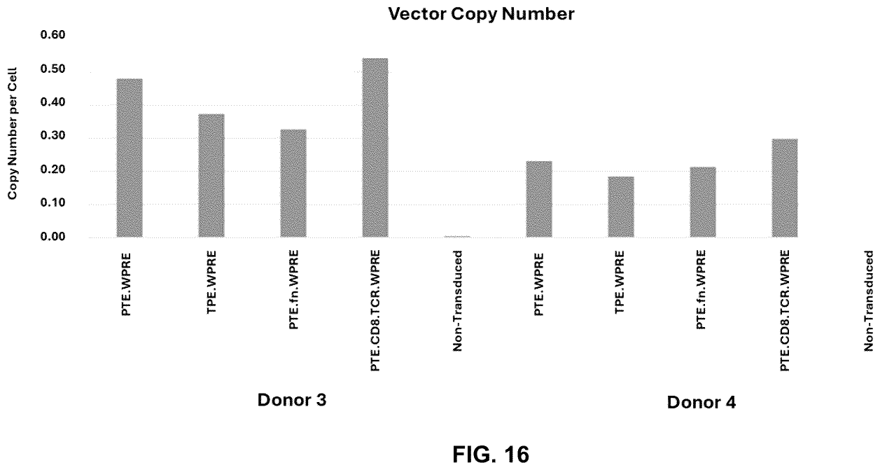

FIG. 16 shows γδ T cells obtained from Donor 3 and Donor 4 transduced with 4-in-1 viral vector containing PTE.CD8.TCR.WPRE results in fewer than 0.6 copy of integrated vector per cell similar to that transduced with PTE.WPRE, TPE.WPRE, and PTE.fn.WPRE. This low copy number of integrated vector per cell is within the limit of safety requirement, i.e., fewer than 5 copy number of integrated vector per cell.

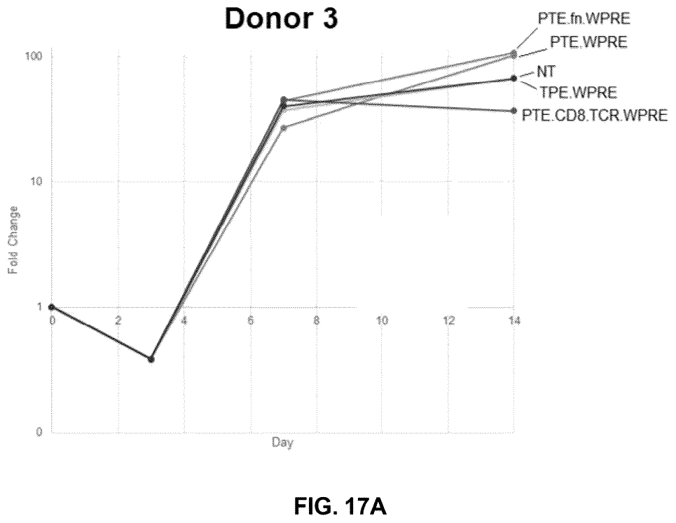

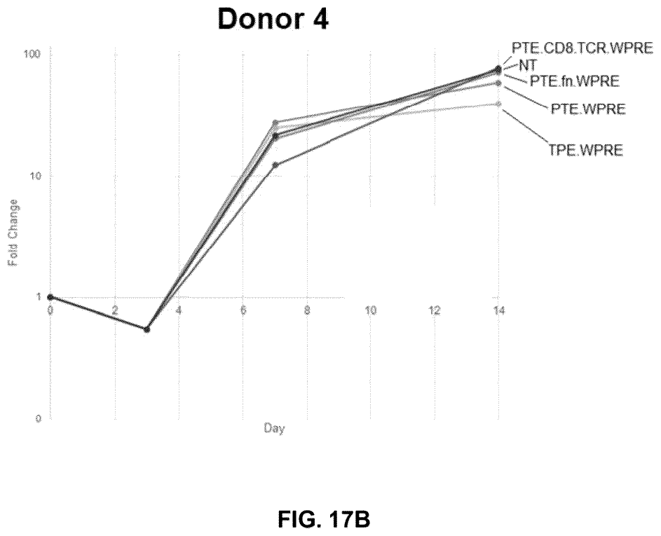

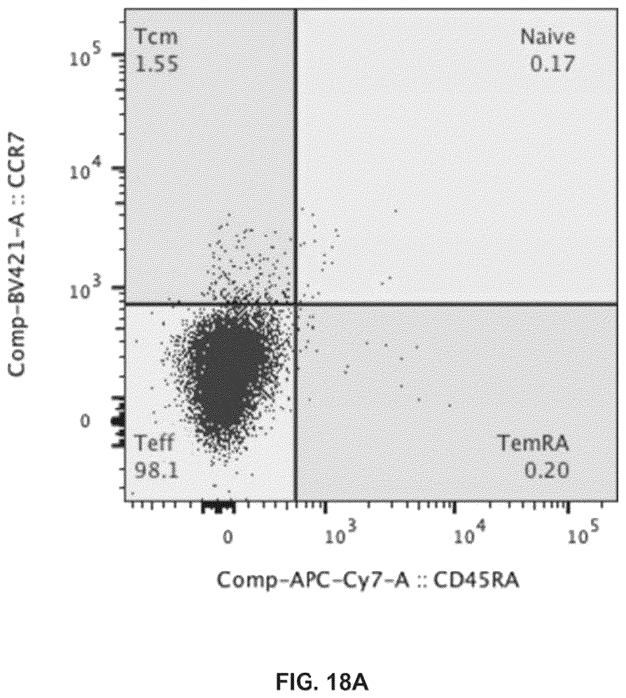

FIGS. 17A and 17B show γδ T cells obtained from Donor 3 and Donor 4, respectively, transduced with 4-in-1 viral vector containing PTE.CD8.TCR.WPRE achieve comparable levels of cell expansion at Day 14 of manufacturing to that transduced with 4-in-1 viral vector containing PTE.WPRE, TPE.WPRE, or PTE.fn.WPRE.

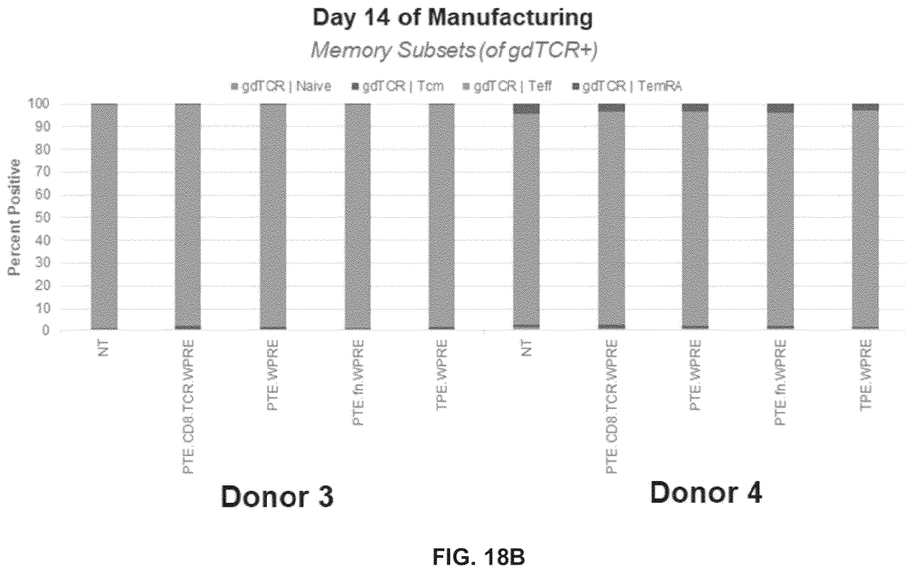

To determine memory cell phenotypes of the transduced γδ T cells, cells were stained by allophycocyanin (APC)-Cy7-tagged anti-CD45RA antibodies and BV421-tagged anti-CCR7 antibodies, followed by FACS analysis to determine the % of Tcm, Naïve T cells, TemRA, and Teff present in the transduced γδ T cells. FIG. 18A shows an example of such an analysis.

FIG. 18B shows γδ T cells obtained from Donor 3 and Donor 4 transduced with 4-in-1 viral vector containing PTE.CD8.TCR.WPRE achieve comparable levels of memory T cell phenotypes at Day 14 of manufacturing to that transduced with 4-in-1 viral vector containing PTE.WPRE, TPE.WPRE, or PTE.fn.WPRE.

### Example 6

Effects on Transgene Expression in Cells Transduced with One 4-In-1 Viral Vector Versus Transduced with Two 2-In-1 Viral Vectors

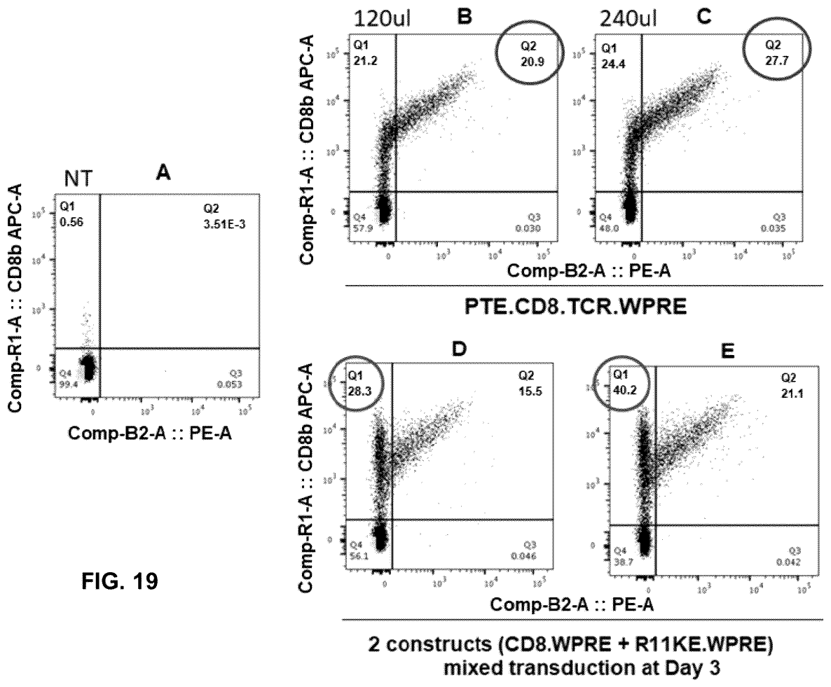

FIG. 19 show more CD8+TCR+γδ T cells resulted from transduction with 4-in-1 lentiviral vector containing PTE.CD8.TCR.WPRE (120 μl) (panel B, 20.9%) than that from transduction with a mixture of a 2-in-1 lentiviral vector containing CD8.WPRE (120 μl) and a 2-in-1 lentiviral vector containing R11KE.WPRE (120 μl) (panel D, 15.5%). On the other hand, more CD8+TCR-γδ T cells resulted from transduction with a mixture of a 2-in-1 lentiviral vector containing CD8.WPRE (120 μl) and a 2-in-1 lentiviral vector containing R11KE.WPRE (120 μl) (panel D, 28.3%) than that transduced with 4-in-1 lentiviral viral vector containing PTE.CD8.TCR.WPRE (panel B, 21.2%). Similarly, more CD8+TCR+γδ T cells resulted from transduction with 4-in-1 lentiviral vector containing PTE.CD8.TCR.WPRE (240 μl) (panel C, 27.7%) than that transduced with a mixture of a 2-in-1 lentiviral vector containing CD8.WPRE (240 μl) and a 2-in-1 lentiviral vector containing R11KE.WPRE (240 μl) (panel E, 21.1%). On the other hand, more CD8+TCR-γδ T cells resulted from transduction with a mixture of a 2-in-1 lentiviral vector containing CD8.WPRE (240 μl) and a 2-in-1 lentiviral vector containing R11KE.WPRE (240 μl) (panel E, 40.2%) than that transduced with 4-in-1 lentiviral vector containing PTE.CD8.TCR.WPRE (panel C, 24.4%). Non-transduced (NT) γδ T cells serves as control (panel A). The 2-color staining was performed using APC-tagged anti-CD8β antibodies and phycoerythrin (PE)-tagged target peptide/MHC complex tetramer. These results suggest that transduction with 4-in-1 lentiviral vector containing sequences encoding CD8αβ and TCRαβ, e.g., PTE.CD8.TCR.WPRE, may result in higher number of CD8+TCR+ T cells than that transduced with a mixture of a 2-in-1 lentiviral vector containing sequences encoding CD8αβ, e.g., CD8.WPRE, and a 2-in-1 lentiviral vector containing sequences encoding TCRαβ, e.g., R11KE.WPRE. On the other hand, transduction with a mixture of a 2-in-1 lentiviral vector containing sequences encoding CD8αβ, e.g., CD8.WPRE, and a 2-in-1 lentiviral vector containing sequences encoding TCRαβ, e.g., R11KE.WPRE, may result in higher number of CD8+TCR-T cells than that transduced with 4-in-1 lentiviral vector containing sequences encoding CD8αβ and TCRαβ, e.g., PTE.CD8.TCR.WPRE.

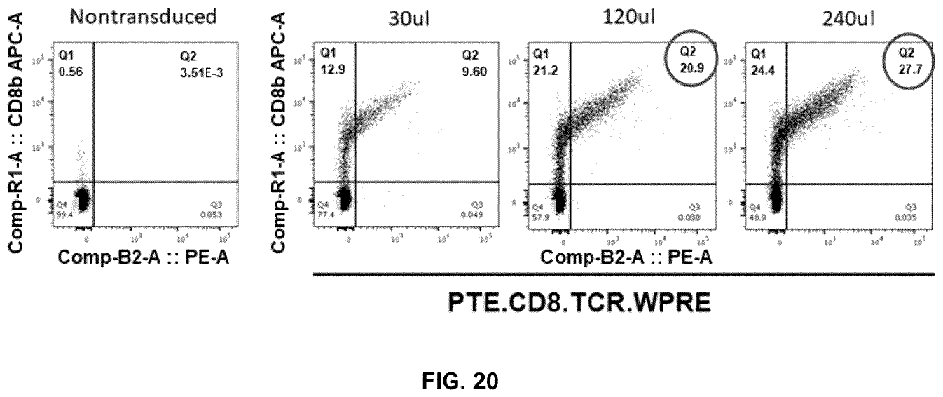

FIG. 20 shows that increasing amount of 4-in-1 viral vector containing PTE.CD8.TCR.WPRE, e.g., 30 μl, 120 μl, and 240 μl, for transduction enhances transduction efficiency, e.g., the % of CD8+TCR+γδ T cells increases, e.g., 9.6% at 30 μl, 20.9% at 120 μl, and 27.7% at 240 μl. Non-transduced γδ T cells serves as control. The 2-color staining was performed using APC-tagged anti-CD8β antibodies and PE-tagged target peptide/MHC complex tetramer.

### Example 7

### Expression of 4-In-1 Constructs in αβ T Cells

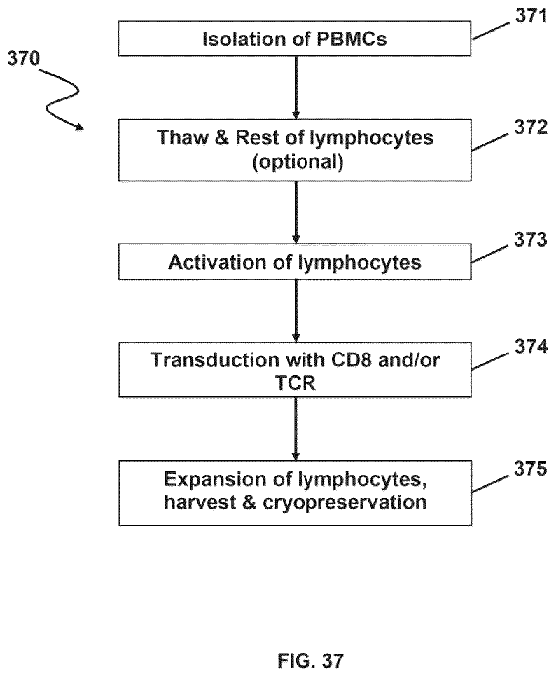

Engineered lymphocytes including engineered αβ T cells expressing recombinant proteins, e.g., CD8αβ and/or TCRαβ, can be manufactured according to the methods disclosed in US 2019/0247433, the content of which is hereby incorporated by reference in its entirety. For example, FIG. 37 shows a T cell manufacturing process 370, which may include isolation of PBMC (371), in which PBMC may be used fresh or stored frozen till ready for use, or may be leukapheresis products, e.g., leukopaks, or may be used as starting materials for T cell manufacturing and selection of lymphocyte populations (e.g., αβ TCR+ T cells, CD8+, CD4+, or both); thaw and rest lymphocytes overnight, e.g., about 16 hours or about 4-6 hours, (372), which may allow apoptotic cells to die off and restore T cell functionality (this step may not be necessary, if fresh materials are used); activation of lymphocytes (373), which may use anti-CD3 and anti-CD28 antibodies (soluble or surface bound, e.g., magnetic or biodegradable beads, antibodies immobilized on culture vessels); transduction with viral vectors containing sequences encoding recombinant proteins, e.g., CD8αβ and/or TCRαβ polypeptides (374), in which the viral vectors may be lentiviral vectors or retroviral vectors, or transfection may be performed by non-viral methods; and expansion of lymphocytes, harvest, and cryopreservation (375), which may be carried out in the presence of cytokine(s), e.g., IL-7 and IL-15, serum (ABS or FBS), and/or cryopreservation media.

### Exogenous CD8 Expression

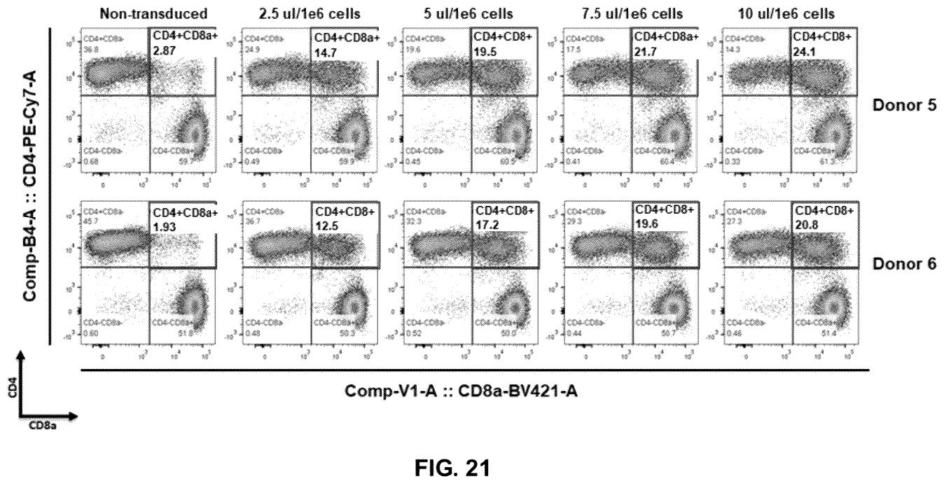

To determine the exogenous CD8 expression in αβ T cells transduced with viral vectors containing 4-in-1 constructs with sequences encoding CD8 and TCR, T cells obtained from Donor 5 and Donor 6 were transduced with increasing amount of LV-PTE.CD8.TCR.WPRE, followed by FACS gated on Lymphocytes<Singlets<Live cells<CD3+ population to detect % CD8α+ cells in CD4+ cells. FIG. 21 shows % CD8α+CD4+ cells from Donor 5 increases from 2.87% (non-transduced) to 14.7% (2.5 μl/1×10⁶ cells), 19.5% (5 μl/1×10⁶ cells), 21.7% (7.5 μl/1×10⁶ cells), and 24.1% (10 μl/1×10⁶ cells); and % CD8α+CD4+ cells from Donor 6 increases from 1.93% (non-transduced) to 12.5% (2.5 μl/1×10⁶ cells), 17.2% (5 μl/1×10⁶ cells), 19.6% (7.5 μl/1×10⁶ cells), and 20.8% (10 μl/1×10⁶ cells).

### Exogenous TCR Expression

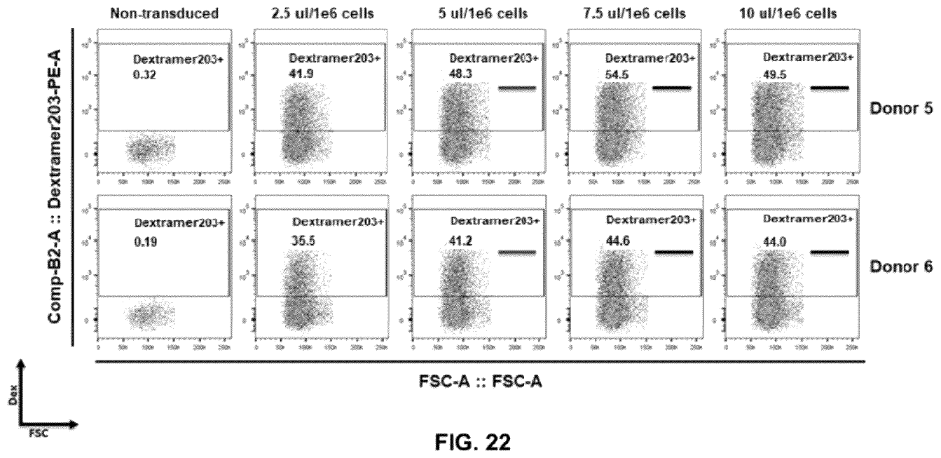

To determine the exogenous TCR expression in αβ T cells transduced with viral vectors containing 4-in-1 constructs with sequences encoding CD8 and TCR, T cells obtained from Donor 5 and Donor 6 were transduced with increasing amount of LV-PTE.CD8.TCR.WPRE, followed by FACS gated on Lymphocytes<Singlets<Live cells<CD3+<CD4+CD8+ population to detect % target peptide/MHC complex Dextramer203+ (i.e., TCR+) cells in CD4+CD8+ cell population. FIG. 22 shows % Dextramer203+ cells from Donor 5 increases from 0.32% (non-transduced) to 41.9% (2.5 μl/1×10⁶ cells), 48.3% (5 μl/1×10⁶ cells), 54.5% (7.5 μl/1×10⁶ cells), and 49.5% (10 μl/1×10⁶ cells); and % Dextramer203+ cells from Donor 6 increases from 0.19% (non-transduced) to 35.5% (2.5 μl/1×10⁶ cells), 41.2% (5 μl/1×10⁶ cells), 44.6% (7.5 μl/1×10⁶ cells), and 44.0% (10 μl/1×10⁶ cells).

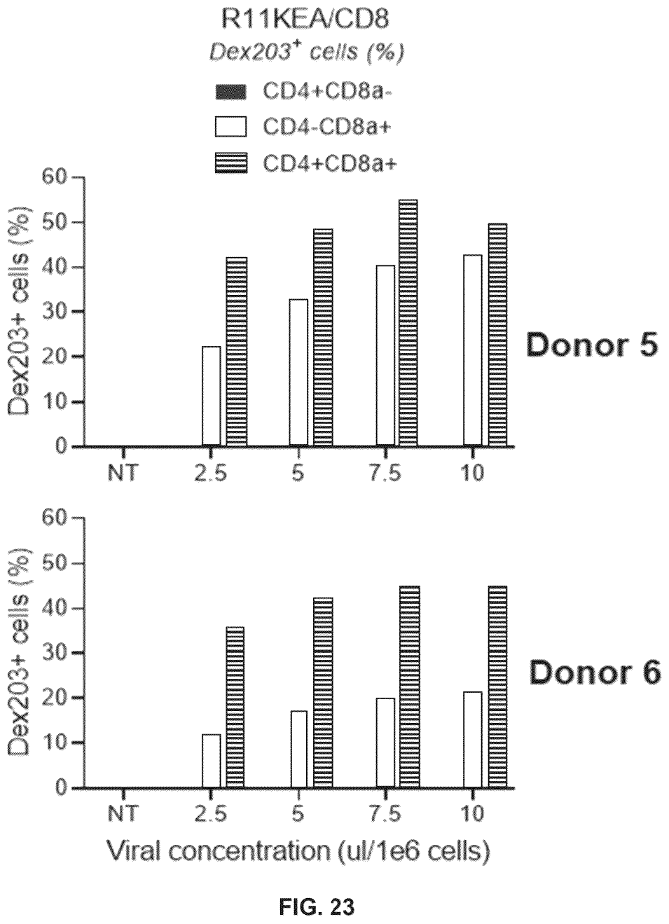

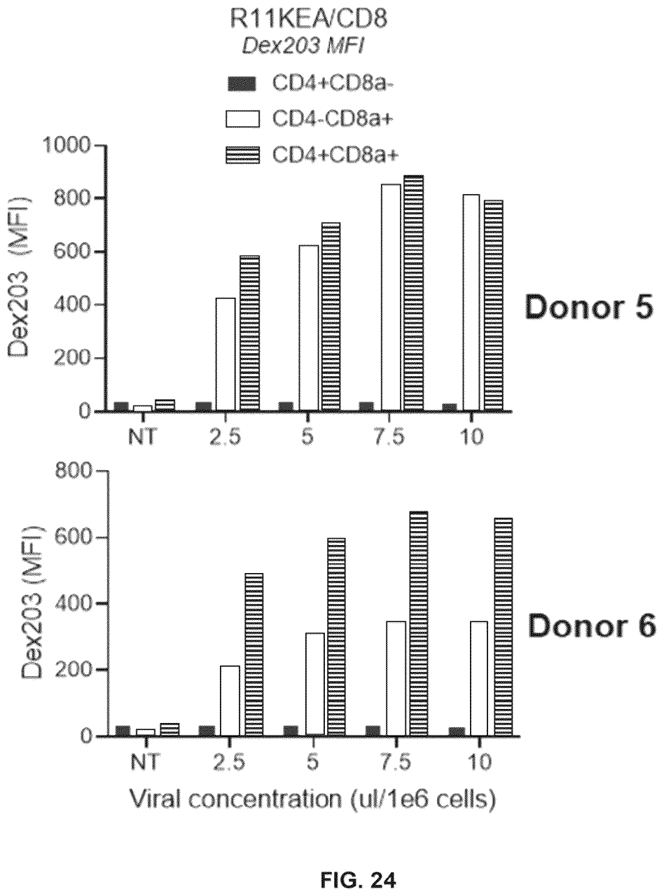

To detect TCR expression in various αβ T cell populations, αβ T cells transduced with LV-PTE.CD8.TCR.WPRE were analyzed by FACS gated on Lymphocytes<Singlets<Live cells<CD3+<CD4+/−CD8+/−. FIG. 23 shows % Dextramer203 (Dex203)+ (i.e., TCR+) cells obtained from Donor 5 (top panel) and Donor 6 (bottom panel) are generally higher in CD4+CD8α+ cell population than that in CD4-CD8α+ cell population. In contrast, % Dex203+ (i.e., TCR+) cells is minimum in CD4+CD8α-cell population. Similarly, FIG. 24 shows % Dextramer203 (Dex203) MFI obtained from Donor 5 (top panel) and Donor 6 (bottom panel) are generally higher in CD4+CD8α+ cell population than that in CD4-CD8α+ cell population. In contrast, % Dex203 MFI is minimum in CD4+CD8α-cell population. These results suggest that exogenous TCR and CD8 encoded by LV-PTE.CD8.TCR.WPRE can be co-expressed in both CD4+ T cells and CD4-T cells.

### Example 8

### Functional Analysis of αβ T Cells Expressing 4-In-1 Construct or TCR Only Construct

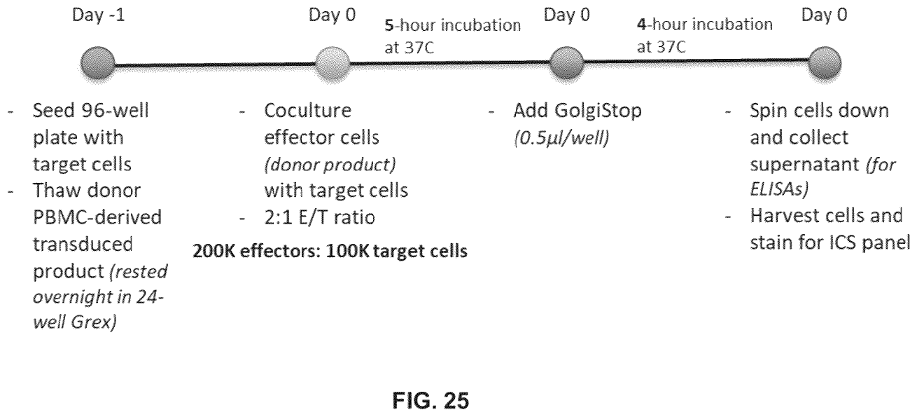

FIG. 25 shows an experimental design for testing functionality of αβ T cells transduced with lentiviral vector (LV) containing 4-in-1 construct, e.g., PTE.CD8.TCR.WPRE (LV-CD8.TCR), or TCR-only construct, e.g., R11KE.WPRE (LV-TCR). Briefly, on Day-1, target cells, e.g., high antigen expressing UACC257+RFP cell line (positive control) and antigen-negative MCF7+GFP cell line (negative control), were seeded in 96-well plates. Donor cell products, e.g., PBMC (obtained from Donors 5, 6, 7, and 8) transduced with LV-CD8.TCR (5 μl/1×10⁶ cells) or LV-TCR (2.5 μl/1×10⁶ cells), were thawed and rested overnight in 24-well G-Rex® gas permeable rapid expansion device. On Day 0, donor cell products (effector cells) were co-cultured with target cells, e.g., UACC257+RFP and MCF7+GFP, at an effector cells to target cells (E/T) ratio of 2:1, e.g., 200,000 effector cells: 100,000 target cells. After 5-hour incubation at 37° C., protein transport inhibitor, e.g., GolgiStop™ (BD Biosciences), was added to each well at 0.5 μl/well, followed by 4-hour of incubation at 37° C. Cells were then centrifuged to collect supernatants for ELISA to detect IFN-γ expression and to harvest cells for staining using intracellular cytokine staining (ICS) panel, e.g., CD3, CD4, CD8, IFN-γ, Granzyme B, and live/dead.

### CD4-CD8+ T Cell Population

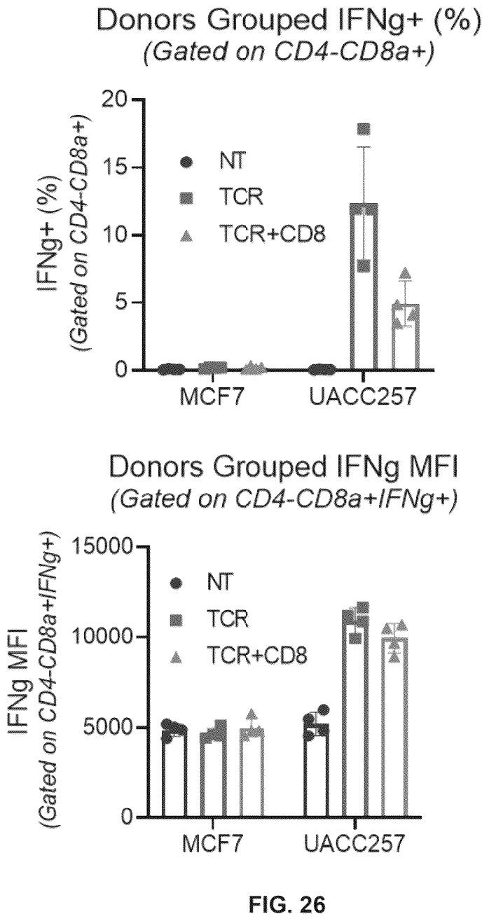

FIG. 26 shows that co-culturing CD4-CD8α+ T cells obtained from grouped donors transduced with LV-TCR (TCR) or LV-CD8.TCR (TCR+CD8) with high-target expressing UACC257 cells resulted in higher % IFN-γ-positive cells (top panel) and higher IFN-γ MFI (bottom panel) than that without transduction (NT). In contrast, no significant difference in % IFN-γ-positive cells and IFN-γ MFI between transduced and non-transduced cells was observed when co-culturing CD4-CD8α+ T cells transduced with LV-TCR (TCR) or LV-CD8.TCR (TCR+CD8) with antigen-negative MCF7. FACS was gated on CD4-CD8α+IFN-γ+ T cells. Non-transduced (NT) cells serve as control. (Effector to target cell ratio=2:1 and Donors grouped N=4). These results suggest that CD4-CD8α+ T cells transduced with LV-TCR or LV-CD8.TCR are functionally active, e.g., by expressing IFN-γ, when contacting high antigen expressing target cells, e.g., UACC257 cells, and the transduced cells have little effect on antigen-negative cells, e.g., MCF7 cells.

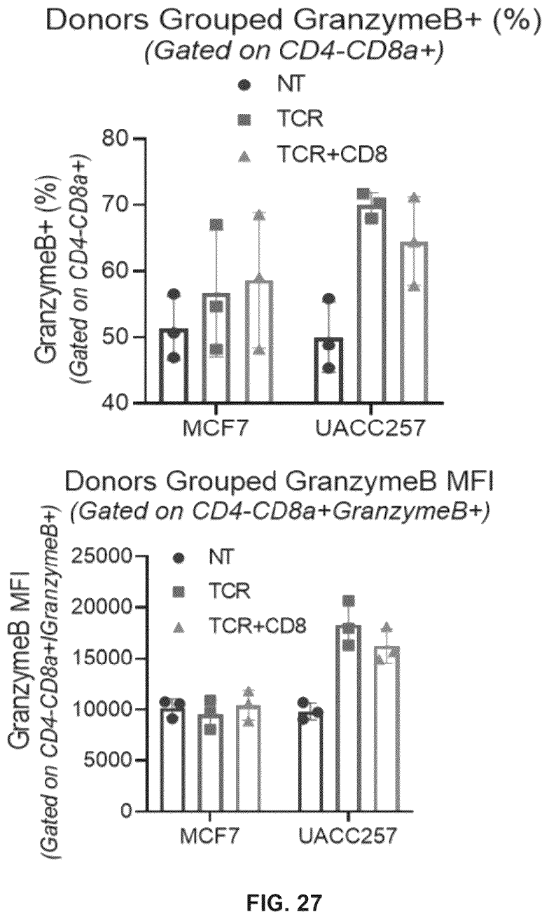

FIG. 27 shows that co-culturing CD4-CD8α+ T cells obtained from grouped donors transduced with LV-TCR (TCR) or LV-CD8.TCR (TCR+CD8) with high-target expressing UACC257 cells resulted in higher % Granzyme B-positive cells (top panel) and higher Granzyme B MFI (bottom panel) than that without transduction (NT). In contrast, no significant difference in % Granzyme B-positive cells and Granzyme B MFI between transduced and non-transduced cells was observed when co-culturing CD4-CD8α+ T cells transduced with LV-TCR (TCR) or LV-CD8.TCR (TCR+CD8) with antigen-negative MCF7. FACS was gated on CD4-CD8α+Granzyme B+ T cells. Non-transduced (NT) cells serve as control. (Effector to target cell ratio=2:1 and Donors grouped N=3). These results suggest that CD4-CD8α+ T cells transduced with LV-TCR or LV-CD8.TCR are functionally active, e.g., by expressing Granzyme B, when contacting high antigen expressing target cells, e.g., UACC257 cells, and the transduced cells may have little effect on antigen-negative cells, e.g., MCF7 cells.

### CD4+CD8+ T Cell Population

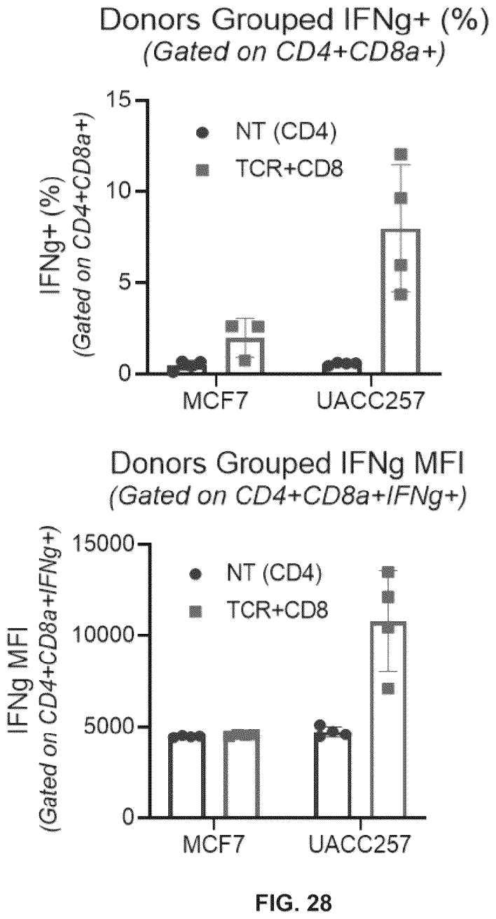

FIG. 28 shows that co-culturing CD4+CD8α+ T cells obtained from grouped donors transduced with LV-CD8.TCR (TCR+CD8) with high-target expressing UACC257 cells resulted in higher % IFN-γ-positive cells (top panel) and higher IFN-γ MFI (bottom panel) than that without transduction (NT). In contrast, no significant difference in % IFN-γ-positive cells and IFN-γ MFI between transduced and non-transduced cells was observed when co-culturing CD4+CD8α+ T cells transduced with LV-CD8.TCR (TCR+CD8) with antigen-negative MCF7. FACS was gated on CD4+CD8α-IFN-γ+ for the NT cells and CD4+CD8α+IFN-γ+ for the LV-CD8.TCR-transduced cells. Non-transduced (NT) cells serve as control. (Effector to target cell ratio=2:1 and Donors grouped N=4). These results suggest that CD4+CD8+ T cells transduced with LV-CD8.TCR are functionally active, e.g., by expressing IFN-γ, when contacting high antigen expressing target cells, e.g., UACC257 cells, and the transduced cells may have little effect on antigen-negative cells, e.g., MCF7 cells.

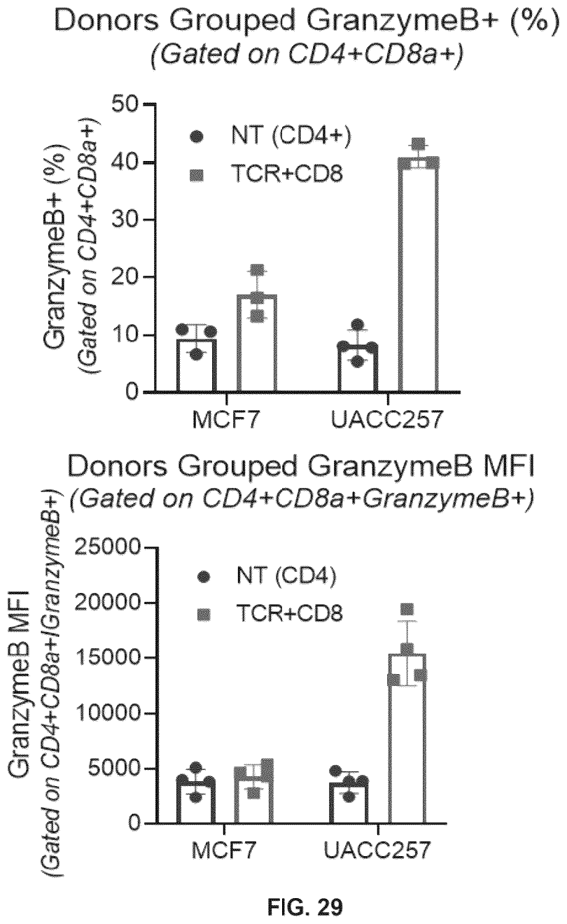

FIG. 29 shows that co-culturing CD4+CD8α+ T cells obtained from grouped donors transduced with LV-CD8.TCR (TCR+CD8) with high-target expressing UACC257 cells resulted in higher % Granzyme B-positive cells (top panel) and higher Granzyme B MFI (bottom panel) than that without transduction (NT). In contrast, no significant difference in % Granzyme B-positive cells and Granzyme B MFI between transduced and non-transduced cells was observed when co-culturing CD4+CD8α+ T cells transduced with LV-CD8.TCR (TCR+CD8) with antigen-negative MCF7. FACS was gated on CD4+CD8α-Granzyme B+ for the NT cells and CD4+CD8α+Granzyme B+ for the LV-CD8.TCR-transduced T cells. Non-transduced (NT) cells serve as control. (Effector to target cell ratio=2:1 and Donors grouped N=3). These results suggest that CD4+CD8α+ T cells transduced with LV-CD8.TCR are functionally active, e.g., by expressing Granzyme B, when contacting high antigen expressing target cells, e.g., UACC257 cells, and the transduced cells may have little effect on antigen-negative cells, e.g., MCF7 cells.

### CD3+ T Cells

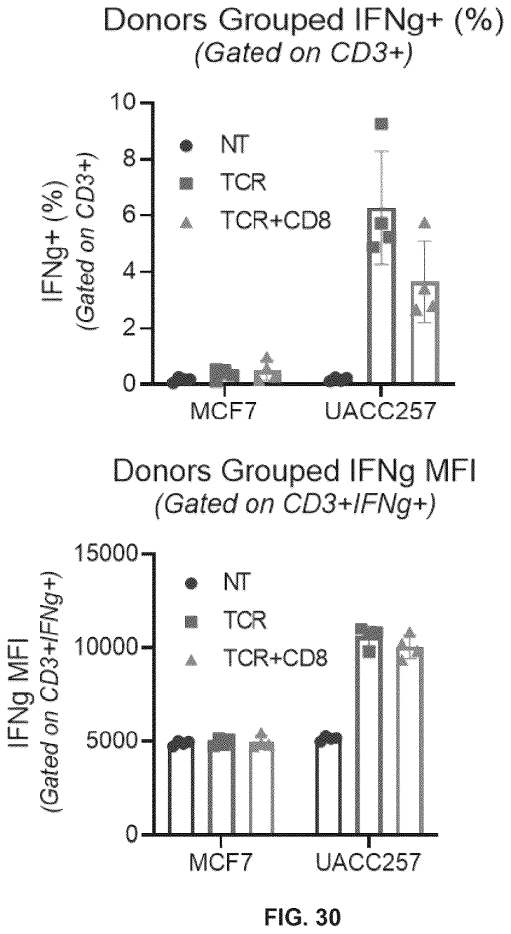

FIG. 30 shows that co-culturing CD3+ T cells obtained from grouped donors transduced with LV-TCR (TCR) or LV-CD8.TCR (TCR+CD8) with high-target expressing UACC257 cells resulted in higher % IFN-γ-positive cells (top panel) and higher IFN-γ MFI (bottom panel) than that without transduction (NT). In contrast, no significant difference in % IFN-γ-positive cells and IFN-γ MFI between transduced and non-transduced cells was observed when co-culturing CD4-CD8α+ T cells transduced with LV-TCR (TCR) or LV-CD8.TCR (TCR+CD8) with antigen-negative MCF7. FACS was gated on CD3+ T cells. Non-transduced (NT) cells serve as control. (Effector to target cell ratio=2:1 and Donors grouped N=4). These results suggest that CD3+ T cells transduced with LV-TCR or LV-CD8.TCR are functionally active, e.g., by expressing IFN-γ, when contacting high antigen expressing target cells, e.g., UACC257 cells, and the transduced cells may have little effect on antigen-negative cells, e.g., MCF7 cells.

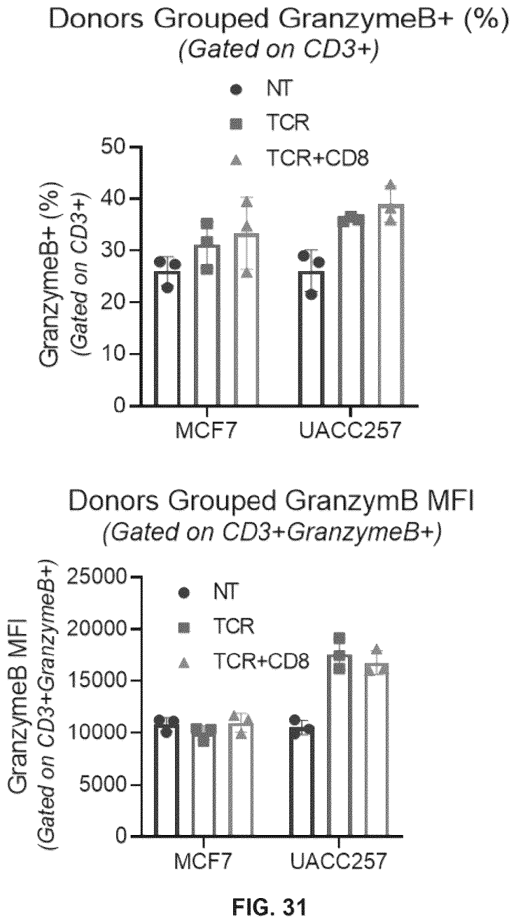

FIG. 31 shows that co-culturing CD3+ T cells obtained from grouped donors transduced with LV-TCR (TCR) or LV-CD8.TCR (TCR+CD8) with high-target expressing UACC257 cells resulted in higher % Granzyme B-positive cells (top panel) and higher Granzyme B MFI (bottom panel) than that without transduction (NT). In contrast, no significant difference in % Granzyme B-positive cells and Granzyme B MFI between transduced and non-transduced cells was observed when co-culturing CD3+ T cells transduced with LV-TCR (TCR) or LV-CD8.TCR (TCR+CD8) with antigen-negative MCF7. FACS was gated on CD3+ T cells. Non-transduced (NT) cells serve as control. (Effector to target cell ratio=2:1 and Donors grouped N=3). These results suggest that CD3+ T cells transduced with LV-TCR or LV-CD8.TCR are functionally active, e.g., by expressing Granzyme B, when contacting high antigen expressing target cells, e.g., UACC257 cells, and the transduced cells may have little effect on antigen-negative cells, e.g., MCF7 cells.

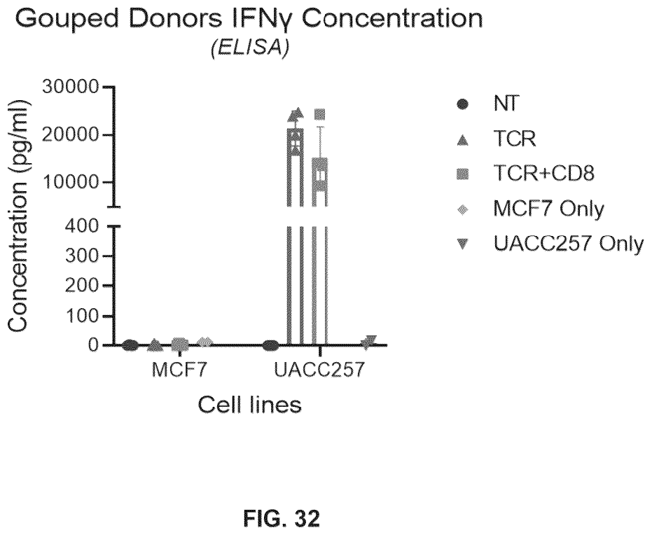

FIG. 32 shows that co-culturing CD3+ T cells obtained from group donors transduced with LV-TCR (TCR) or LV-CD8.TCR (TCR+CD8) with high-target expressing UACC257 cells resulted in higher levels of IFN-γ secretion than that without transduction (NT), MCF7 cells only, and UACC257 cells only. In contrast, no significant difference in the levels of IFN-γ secretion between transduced and non-transduced cells was observed when co-culturing CD4-CD8α+ T cells transduced with LV-TCR (TCR) or LV-CD8.TCR (TCR+CD8) with antigen-negative MCF7. (Effector to target cell ratio=2:1 and Donors grouped N=4). These results suggest that CD3+ T cells transduced with LV-TCR or LV-CD8.TCR are functionally active, e.g., by secreting IFN-γ, when contacting high antigen expressing target cells, e.g., UACC257 cells, and the transduced cells may have little effect on antigen-negative cells, e.g., MCF7 cells.

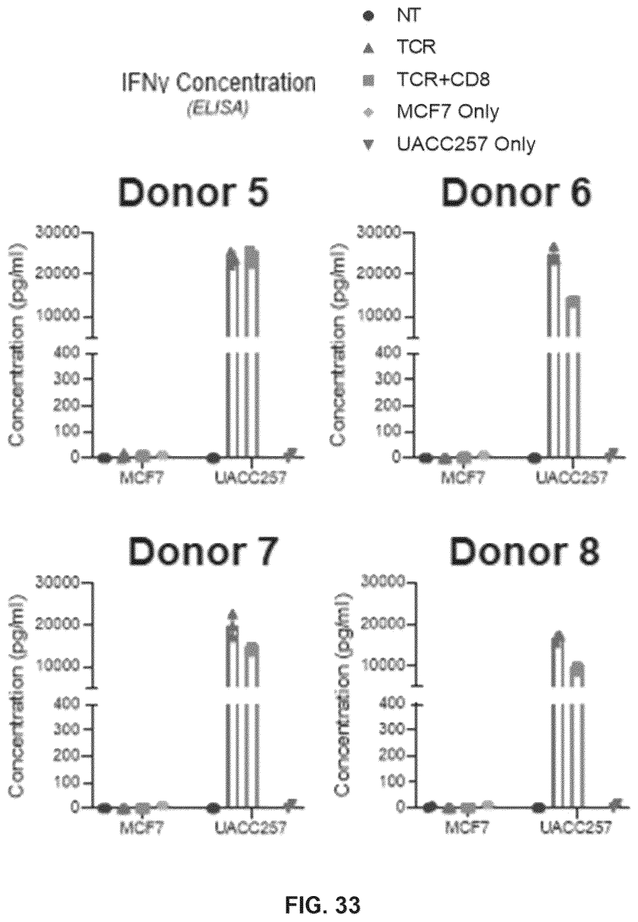

FIG. 33 shows that co-culturing CD3+ T cells obtained from individual Donors 5, 6, 7, and 8 transduced with LV-TCR (TCR) or LV-CD8.TCR (TCR+CD8) with high-target expressing UACC257 cells resulted in higher levels of IFN-γ secretion than that without transduction (NT), MCF7 cells only, and UACC257 cells only. In contrast, no significant difference in the levels of IFN-γ secretion between transduced and non-transduced cells was observed when co-culturing CD4-CD8α+ T cells transduced with LV-TCR (TCR) or LV-CD8.TCR (TCR+CD8) with antigen-negative MCF7. (Effector to target cell ratio=2:1). These results suggest that CD3+ T cells obtained from individual donors transduced with LV-TCR or LV-CD8.TCR are functionally active, e.g., by secreting IFN-γ, when contacting high antigen expressing target cells, e.g., UACC257 cells, and the transduced cells may have little effect on antigen-negative cells, e.g., MCF7 cells.

### Example 9

### Effect of Statins on the Expression of T Cell Activation Markers

To determine the effect of statins on the expression of T cell activation markers, T cells were treated with statins, e.g., atorvastatin, pravastatin, or rosuvastatin, followed by FACS analysis to measure the expression of T cell activation markers, e.g., CD25, CD69, and hLDLR.

### CD4+ T Cell Population

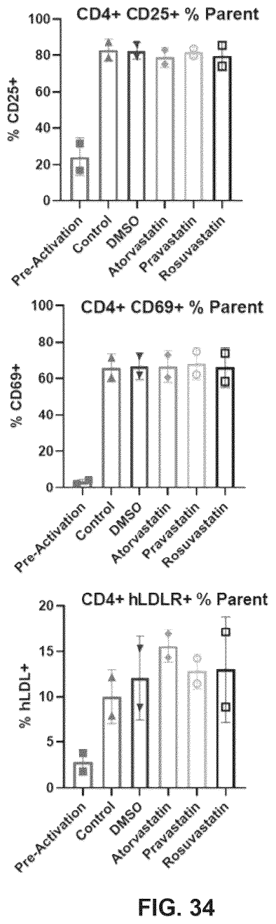

FIG. 34 shows % CD25+ cells (top panel), % CD69+ cells (middle panel), and % hLDLR+ cells (bottom panel) in CD3+CD4+ T cells treated with atorvastatin, pravastatin, or rosuvastatin. Pre-activated cells, cells activated without statin or DMSO (control), and DMSO serve as controls. These results show that, while atorvastatin, pravastatin, and rosuvastatin have little effect on the % CD4+CD25+ cells and the % CD4+CD69+ cells, statins, e.g., atorvastatin, may increase the % CD4+hLDLR+ cells. FACS was gated on Lymphocytes>Singlets>Live/Dead>CD3+>CD4+.

### CD8+ T Cell Population

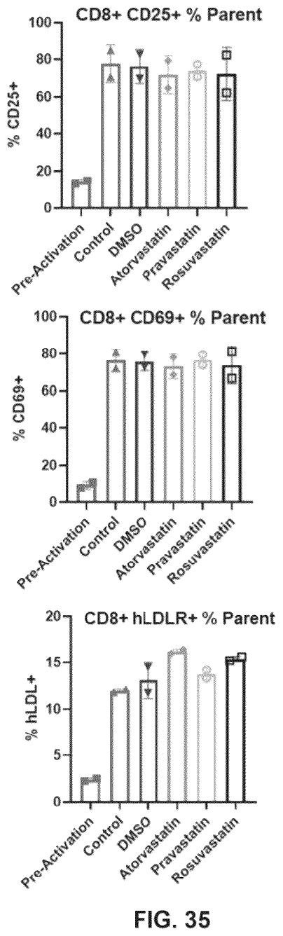

FIG. 35 shows % CD25+ cells (top panel), % CD69+ cells (middle panel), and % hLDLR+ cells (bottom panel) in CD3+CD8+ T cells treated with atorvastatin, pravastatin, or rosuvastatin. Pre-activated cells, cells activated without statin or DMSO (control), and DMSO serve as controls. These results show that, while atorvastatin, pravastatin, and rosuvastatin have little effect on the % CD8+CD25+ cells and the % CD8+CD69+ cells, statins, e.g., atorvastatin, may increase the % CD8+hLDLR+ cells. FACS was gated on Lymphocytes>Singlets>Live/Dead>CD3+>CD8+.

### Example 10

### Effect of WPRE on Lentiviral Titers

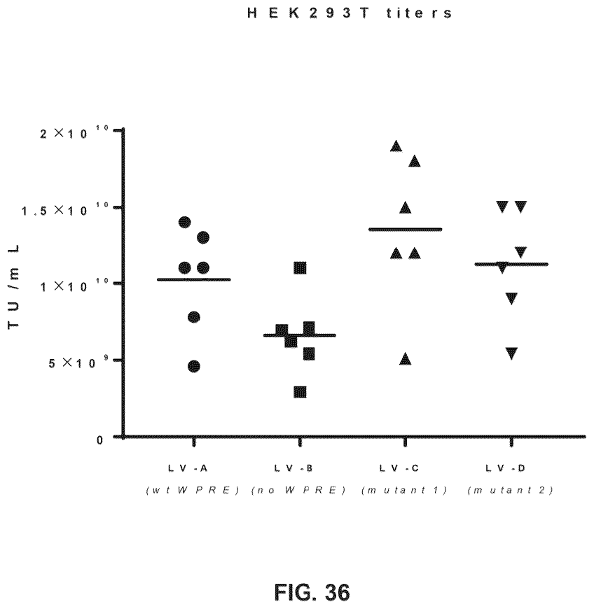

To determine the effect of WPRE on lentiviral titers, lentiviral vectors (LV) containing wild type (wt) WPRE (SEQ ID NO: 9) (LV-A), no WPRE (LV-B), WPREmut1 (SEQ ID NO: 265) (LV-C), or WPREmut2 (SEQ ID NO: 266) (LV-D) were generated. HEK293T cells were transfected with LV-A, LV-B, LV-C, or LV-D followed by titer determination using methods known in the art. FIG. 36 shows the titers of these lentiviral vectors are in the order of LV-C>LV-D≥LV-A>LV-B. These results suggest that WPREmut1 and WPREmut2 may be useful to improving lentiviral vector production.

Advantages of the present disclosure may include generation of viral vectors that co-express multiple transgenes, e.g., 4 polypeptides, in a single vector, and generation of γδ T cells that co-express TCRαβ and CD8αβ as safe and target-specific “off-the-shelf” T cell products for adoptive cellular therapy.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

What is claimed is:

1. A T cell transduced with a vector comprising a nucleotide sequence S1 encoding a CD8α polypeptide, a nucleotide sequence S2 encoding a CD8β polypeptide, a nucleotide sequence S3 encoding a T cell receptor (TCR)α polypeptide, and a nucleotide sequence S4 encoding a TCRβ polypeptide, wherein the nucleotide sequences are arranged in tandem in a 5′ to 3′ orientation of S2-S1-S4-S3.

2. The T cell of claim 1, further comprising a nucleotide sequence S5 encoding a 2A peptide and a nucleotide sequence S6 encoding a linker peptide, wherein S5 and S6 are positioned between S1 and S2, S1 and S4, and/or S3 and S4.

3. The T cell of claim 2, wherein the 2A peptide is selected from P2A (SEQ ID NO: 3), T2A (SEQ ID NO: 4), E2A (SEQ ID NO: 5), or F2A (SEQ ID NO: 6).

4. The T cell of claim 2, wherein the linker peptide is GSG or SGSG (SEQ ID NO: 8).

5. The T cell of claim 1, further comprising a nucleotide sequence S7 encoding a furin peptide (SEQ ID NO: 2), wherein S7 is positioned between S1 and S2, S1 and S4, and/or S3 and S4.

6. The T cell of claim 1, wherein the T cell comprises an αβ T cell, a γδ T cell, and/or a natural killer T cell.

7. A composition comprising the T cell of claim 1.

8. The composition of claim 7, further comprising an adjuvant selected from anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and IL-23.

9. The composition of claim 8, wherein the adjuvant is IL-2.

10. The composition of claim 8, wherein the adjuvant is IL-15.

11. A method of treating a patient who has cancer, comprising administering to the patient a composition of claim 7, and
wherein the T cell kill cancer cells that present a peptide in a complex with an MHC molecule on the surface,
wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

12. A method of eliciting an immune response in a patient who has cancer, comprising administering to the patient a composition of claim 7,
wherein the T cell kill cancer cells that present a peptide in a complex with an MHC molecule on the surface,
wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

13. A method of preparing a T cell for immunotherapy, comprising
activating the T cell in the presence of a statin,
transducing the activated T cell with a vector comprising a nucleotide sequence S1 encoding a CD8α polypeptide, a nucleotide sequence S2 encoding a CD8β polypeptide, a nucleotide sequence S3 encoding a T cell receptor (TCR) a polypeptide, and a nucleotide sequence S4 encoding a TCRβ polypeptide,
wherein the nucleotide sequences are arranged in tandem in a 5′ to 3′ orientation of S2-S1-S4-S3, and
expanding the transduced T cell.

14. The method of claim 13, further comprising a nucleotide sequence S5 encoding a 2A peptide and a nucleotide sequence S6 encoding a linker peptide, wherein S5 and S6 are positioned between S1 and S2, S1 and S4, and/or S3 and S4.

15. The method of claim 14, wherein the 2A peptide is selected from P2A (SEQ ID NO: 3), T2A (SEQ ID NO: 4), E2A (SEQ ID NO: 5), or F2A (SEQ ID NO: 6).

16. The method of claim 14, wherein the linker peptide is GSG or SGSG (SEQ ID NO: 8).

17. The method of claim 13, further comprising a nucleotide sequence S7 encoding a furin peptide (SEQ ID NO: 2), wherein S7 is positioned between S1 and S2, S1 and S4, and/or S3 and S4.

18. The method of claim 13, wherein the T cell comprises an αβ T cell, a γδ T cell, and/or a natural killer T cell.

19. The method of claim 13, wherein the vector is pseudotyped with an envelope protein of vesicular stomatitis virus (VSV-G).

20. The method of claim 13, wherein the statin is selected from atorvastatin, cerivastatin, dalvastatin, fluindostatin, fluvastatin, mevastatin, pravastatin, simvastatin, velostatin, and rosuvastatin.


SEQUENCE LISTING (266 sequences)

SEQ ID NO: 1 - DNA, 367 bp, synthetic construct
  misc_feature 1..367: note = murine stem cell virus promoter
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcact

SEQ ID NO: 2 - protein, 4 aa, synthetic construct
  REGION 1..4: note = Furin
RAKR

SEQ ID NO: 3 - protein, 19 aa, synthetic construct
  REGION 1..19: note = P2A peptide
ATNFSLLKQAGDVEENPGP

SEQ ID NO: 4 - protein, 18 aa, synthetic construct
  REGION 1..18: note = T2A peptide
EGRGSLLTCGDVEENPGP

SEQ ID NO: 5 - protein, 20 aa, synthetic construct
  REGION 1..20: note = E2A peptide
QCTNYALLKLAGDVESNPGP

SEQ ID NO: 6 - protein, 22 aa, synthetic construct
  REGION 1..22: note = F2A peptide
VKQTLNFDLLKLAGDVESNPGP

SEQ ID NO: 7 - skipped

SEQ ID NO: 8 - protein, 4 aa, synthetic construct
  REGION 1..4: note = Linker peptide
SGSG

SEQ ID NO: 9 - DNA, 607 bp, synthetic construct
  misc_feature 1..607: note = WPRE
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 10 - DNA, 21 bp, synthetic construct
  misc_feature 1..21: note = X protein promoter
  source 1..21: mol_type = other DNA
ggggaagctgacgtcctttcc

SEQ ID NO: 11 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 12 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 13 - protein, 273 aa, synthetic construct
  REGION 1..273: note = R11KEA alpha chain
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 311 aa, synthetic construct
  REGION 1..311: note = R11KE beta chain
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 15 - protein, 274 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 314 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 272 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 310 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 277 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 313 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 271 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESKETRLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 317 aa, Homo sapiens
MLLLLLLLGPGISLLLPGSLAGSGLGAWSQHPSVWICKSGTSVKIECRSLDFQATTMFWYRQFPKQSLMLMATSNEGSKATYEQGVEKDKFLINHASLTLSTLTVTSAHPEDSSFYICSARAGGHEQFFGPGTRLTVLEDLKNVFPPEVAVFEPSEAEISHTQKATLVCLATGFYPDHVELSWVWNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 271 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFHDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 308 aa, Homo sapiens
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVASAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 25 - protein, 274 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 308 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 271 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 314 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLTSGGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 270 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 314 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTSGGLSGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 272 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 312 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGSPDGNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 279 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYNSYAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 310 aa, Homo sapiens
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLDGTSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 35 - protein, 271 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCLIGASGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 310 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYFGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 37 - protein, 276 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 311 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASTTPDGTDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 39 - protein, 274 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 310 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRAEGGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 273 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASPTGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 311 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLGGASQEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 43 - protein, 266 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 309 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - protein, 277 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFGYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 311 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSNEGQGWEAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 268 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVHNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 322 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLGQGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 276 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 323 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGTSGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 273 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 311 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRLPSRTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 270 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 321 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGLGTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 55 - protein, 273 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 311 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 57 - protein, 275 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 311 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 59 - protein, 274 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 309 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 273 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 314 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 63 - protein, 277 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 313 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - protein, 206 aa, Homo sapiens
IMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 311 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 67 - protein, 274 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 319 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 69 - protein, 273 aa, synthetic construct; identical to SEQ ID NO: 13
  REGION 1..273: note = R11P3D3KE alpha chain

SEQ ID NO: 70 - protein, 243 aa, synthetic construct
  REGION 1..243: note = R11P3D3KE beta chain
NNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 71 - protein, 223 aa, Homo sapiens
TYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 72 - protein, 307 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 73 - protein, 270 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 74 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 75 - protein, 272 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 271 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 310 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 79 - protein, 270 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 311 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 81 - protein, 281 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 311 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 272 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 320 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 273 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 312 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 87 - protein, 276 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 309 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 89 - protein, 144 aa, Homo sapiens
VKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 307 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - DNA, 4445 bp, synthetic construct
  misc_feature 1..4445: note = PTE WPRE
  source 1..4445: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgccaccaatttcagcctgctgaaacaggccggcgacgtggaagagaaccctggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagatctggcagcggcgagggcagaggcagcctgctgacctgcggcgacgtggaggagaaccccggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 92 - DNA, 4445 bp, synthetic construct
  misc_feature 1..4445: note = TPE WPRE
  source 1..4445: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagatctggcagcggcgccaccaatttcagcctgctgaaacaggccggcgacgtggaagagaaccctggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 93 - DNA, 4430 bp, synthetic construct
  misc_feature 1..4430: note = PTE fn WPRE
  source 1..4430: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggatctggcagcggcgccaccaatttcagcctgctgaaacaggccggcgacgtggaagagaaccctggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagaggcagcggcgagggcagaggcagcctgctgacctgcggcgacgtggaggagaaccccggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 94 - DNA, 4445 bp, synthetic construct
  misc_feature 1..4445: note = PTE CD8 TCR WPRE
  source 1..4445: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 95 - DNA, 2849 bp, synthetic construct
  misc_feature 1..2849: note = R11KE WPRE
  source 1..2849: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgccaccaatttcagcctgctgaaacaggccggcgacgtggaagagaaccctggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 96 - DNA, 2531 bp, synthetic construct
  misc_feature 1..2531: note = CD8 WPRE
  source 1..2531: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 97 - protein, 559 aa, synthetic construct
  REGION 1..559: note = RD114TR
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

SEQ ID NO: 98 - protein, 10 aa, Homo sapiens
YLYDSETKNA

SEQ ID NO: 99 - protein, 9 aa, Homo sapiens
HLMDQPLSV

SEQ ID NO: 100 - protein, 9 aa, Homo sapiens
GLLKKINSV

SEQ ID NO: 101 - protein, 9 aa, Homo sapiens
FLVDGSSAL

SEQ ID NO: 102 - protein, 10 aa, Homo sapiens
FLFDGSANLV

SEQ ID NO: 103 - protein, 9 aa, Homo sapiens
FLYKIIDEL

SEQ ID NO: 104 - protein, 11 aa, Homo sapiens
FILDSAETTTL

SEQ ID NO: 105 - protein, 9 aa, Homo sapiens
SVDVSPPKV

SEQ ID NO: 106 - protein, 8 aa, Homo sapiens
VADKIHSV

SEQ ID NO: 107 - protein, 9 aa, Homo sapiens
IVDDLTINL

SEQ ID NO: 108 - protein, 9 aa, Homo sapiens
GLLEELVTV

SEQ ID NO: 109 - protein, 10 aa, Homo sapiens
TLDGAAVNQV

SEQ ID NO: 110 - protein, 10 aa, Homo sapiens
SVLEKEIYSI

SEQ ID NO: 111 - protein, 9 aa, Homo sapiens
LLDPKTIFL

SEQ ID NO: 112 - protein, 9 aa, Homo sapiens
YTFSGDVQL

SEQ ID NO: 113 - protein, 9 aa, Homo sapiens
YLMDDFSSL

SEQ ID NO: 114 - protein, 9 aa, Homo sapiens
KVWSDVTPL

SEQ ID NO: 115 - protein, 11 aa, Homo sapiens
LLWGHPRVALA

SEQ ID NO: 116 - protein, 11 aa, Homo sapiens
KIWEELSVLEV

SEQ ID NO: 117 - protein, 9 aa, Homo sapiens
LLIPFTIFM

SEQ ID NO: 118 - protein, 9 aa, Homo sapiens
FLIENLLAA

SEQ ID NO: 119 - protein, 11 aa, Homo sapiens
LLWGHPRVALA

SEQ ID NO: 120 - protein, 9 aa, Homo sapiens
FLLEREQLL

SEQ ID NO: 121 - protein, 9 aa, Homo sapiens
SLAETIFIV

SEQ ID NO: 122 - protein, 9 aa, Homo sapiens
TLLEGISRA

SEQ ID NO: 123 - protein, 9 aa, Homo sapiens
KIQEILTQV

SEQ ID NO: 124 - protein, 10 aa, Homo sapiens
VIFEGEPMYL

SEQ ID NO: 125 - protein, 9 aa, Homo sapiens
SLFESLEYL

SEQ ID NO: 126 - protein, 9 aa, Homo sapiens
SLLNQPKAV

SEQ ID NO: 127 - protein, 9 aa, Homo sapiens
GLAEFQENV

SEQ ID NO: 128 - protein, 9 aa, Homo sapiens
KLLAVIHEL

SEQ ID NO: 129 - protein, 9 aa, Homo sapiens
TLHDQVHLL

SEQ ID NO: 130 - protein, 11 aa, Homo sapiens
TLYNPERTITV

SEQ ID NO: 131 - protein, 9 aa, Homo sapiens
KLQEKIQEL

SEQ ID NO: 132 - protein, 10 aa, Homo sapiens
SVLEKEIYSI

SEQ ID NO: 133 - protein, 11 aa, Homo sapiens
RVIDDSLVVGV

SEQ ID NO: 134 - protein, 9 aa, Homo sapiens
VLFGELPAL

SEQ ID NO: 135 - protein, 9 aa, Homo sapiens
GLVDIMVHL

SEQ ID NO: 136 - protein, 9 aa, Homo sapiens
FLNAIETAL

SEQ ID NO: 137 - protein, 9 aa, Homo sapiens
ALLQALMEL

SEQ ID NO: 138 - protein, 9 aa, Homo sapiens
ALSSSQAEV

SEQ ID NO: 139 - protein, 11 aa, Homo sapiens
SLITGQDLLSV

SEQ ID NO: 140 - protein, 9 aa, Homo sapiens
QLIEKNWLL

SEQ ID NO: 141 - protein, 9 aa, Homo sapiens
LLDPKTIFL

SEQ ID NO: 142 - protein, 9 aa, Homo sapiens
RLHDENILL

SEQ ID NO: 143 - protein, 9 aa, Homo sapiens
YTFSGDVQL

SEQ ID NO: 144 - protein, 9 aa, Homo sapiens
GLPSATTTV

SEQ ID NO: 145 - protein, 11 aa, Homo sapiens
GLLPSAESIKL

SEQ ID NO: 146 - protein, 9 aa, Homo sapiens
KTASINQNV

SEQ ID NO: 147 - protein, 9 aa, Homo sapiens
SLLQHLIGL

SEQ ID NO: 148 - protein, 9 aa, Homo sapiens
YLMDDFSSL

SEQ ID NO: 149 - protein, 9 aa, Homo sapiens
LMYPYIYHV

SEQ ID NO: 150 - protein, 9 aa, Homo sapiens
KVWSDVTPL

SEQ ID NO: 151 - protein, 11 aa, Homo sapiens
LLWGHPRVALA

SEQ ID NO: 152 - protein, 9 aa, Homo sapiens
VLDGKVAVV

SEQ ID NO: 153 - protein, 9 aa, Homo sapiens
GLLGKVTSV

SEQ ID NO: 154 - protein, 9 aa, Homo sapiens
KMISAIPTL

SEQ ID NO: 155 - protein, 11 aa, Homo sapiens
GLLETTGLLAT

SEQ ID NO: 156 - protein, 9 aa, Homo sapiens
TLNTLDINL

SEQ ID NO: 157 - protein, 9 aa, Homo sapiens
VIIKGLEEI

SEQ ID NO: 158 - protein, 9 aa, Homo sapiens
YLEDGFAYV

SEQ ID NO: 159 - protein, 11 aa, Homo sapiens
KIWEELSVLEV

SEQ ID NO: 160 - protein, 9 aa, Homo sapiens
LLIPFTIFM

SEQ ID NO: 161 - protein, 10 aa, Homo sapiens
ISLDEVAVSL

SEQ ID NO: 162 - protein, 10 aa, Homo sapiens
KISDFGLATV

SEQ ID NO: 163 - protein, 11 aa, Homo sapiens
KLIGNIHGNEV

SEQ ID NO: 164 - protein, 9 aa, Homo sapiens
ILLSVLHQL

SEQ ID NO: 165 - protein, 9 aa, Homo sapiens
LDSEALLTL

SEQ ID NO: 166 - protein, 13 aa, Homo sapiens
VLQENSSDYQSNL

SEQ ID NO: 167 - protein, 11 aa, Homo sapiens
HLLGEGAFAQV

SEQ ID NO: 168 - protein, 9 aa, Homo sapiens
SLVENIHVL

SEQ ID NO: 169 - protein, 9 aa, Homo sapiens
YTFSGDVQL

SEQ ID NO: 170 - protein, 9 aa, Homo sapiens
SLSEKSPEV

SEQ ID NO: 171 - protein, 10 aa, Homo sapiens
AMFPDTIPRV

SEQ ID NO: 172 - protein, 9 aa, Homo sapiens
FLIENLLAA

SEQ ID NO: 173 - protein, 9 aa, Homo sapiens
FTAEFLEKV

SEQ ID NO: 174 - protein, 9 aa, Homo sapiens
ALYGNVQQV

SEQ ID NO: 175 - protein, 9 aa, Homo sapiens
LFQSRIAGV

SEQ ID NO: 176 - protein, 11 aa, Homo sapiens
ILAEEPIYIRV

SEQ ID NO: 177 - protein, 9 aa, Homo sapiens
FLLEREQLL

SEQ ID NO: 178 - protein, 10 aa, Homo sapiens
LLLPLELSLA

SEQ ID NO: 179 - protein, 9 aa, Homo sapiens
SLAETIFIV

SEQ ID NO: 180 - protein, 11 aa, Homo sapiens
AILNVDEKNQV

SEQ ID NO: 181 - protein, 9 aa, Homo sapiens
RLFEEVLGV

SEQ ID NO: 182 - protein, 9 aa, Homo sapiens
YLDEVAFML

SEQ ID NO: 183 - protein, 11 aa, Homo sapiens
KLIDEDEPLFL

SEQ ID NO: 184 - protein, 9 aa, Homo sapiens
KLFEKSTGL

SEQ ID NO: 185 - protein, 11 aa, Homo sapiens
SLLEVNEASSV

SEQ ID NO: 186 - protein, 10 aa, Homo sapiens
GVYDGREHTV

SEQ ID NO: 187 - protein, 10 aa, Homo sapiens
GLYPVTLVGV

SEQ ID NO: 188 - protein, 9 aa, Homo sapiens
ALLSSVAEA

SEQ ID NO: 189 - protein, 9 aa, Homo sapiens
TLLEGISRA

SEQ ID NO: 190 - protein, 9 aa, Homo sapiens
SLIEESEEL

SEQ ID NO: 191 - protein, 9 aa, Homo sapiens
ALYVQAPTV

SEQ ID NO: 192 - protein, 10 aa, Homo sapiens
KLIYKDLVSV

SEQ ID NO: 193 - protein, 9 aa, Homo sapiens
ILQDGQFLV

SEQ ID NO: 194 - protein, 9 aa, Homo sapiens
SLLDYEVSI

SEQ ID NO: 195 - protein, 9 aa, Homo sapiens
LLGDSSFFL

SEQ ID NO: 196 - protein, 10 aa, Homo sapiens
VIFEGEPMYL

SEQ ID NO: 197 - protein, 9 aa, Homo sapiens
ALSYILPYL

SEQ ID NO: 198 - protein, 9 aa, Homo sapiens
FLFVDPELV

SEQ ID NO: 199 - protein, 11 aa, Homo sapiens
SEWGSPHAAVP

SEQ ID NO: 200 - protein, 9 aa, Homo sapiens
ALSELERVL

SEQ ID NO: 201 - protein, 9 aa, Homo sapiens
SLFESLEYL

SEQ ID NO: 202 - protein, 9 aa, Homo sapiens
KVLEYVIKV

SEQ ID NO: 203 - protein, 10 aa, Homo sapiens
VLLNEILEQV

SEQ ID NO: 204 - protein, 9 aa, Homo sapiens
SLLNQPKAV

SEQ ID NO: 205 - protein, 9 aa, Homo sapiens
KMSELQTYV

SEQ ID NO: 206 - protein, 11 aa, Homo sapiens
ALLEQTGDMSL

SEQ ID NO: 207 - protein, 11 aa, Homo sapiens
VIIKGLEEITV

SEQ ID NO: 208 - protein, 9 aa, Homo sapiens
KQFEGTVEI

SEQ ID NO: 209 - protein, 9 aa, Homo sapiens
KLQEEIPVL

SEQ ID NO: 210 - protein, 9 aa, Homo sapiens
GLAEFQENV

SEQ ID NO: 211 - protein, 9 aa, Homo sapiens
NVAEIVIHI

SEQ ID NO: 212 - protein, 9 aa, Homo sapiens
ALAGIVTNV

SEQ ID NO: 213 - protein, 12 aa, Homo sapiens
NLLIDDKGTIKL

SEQ ID NO: 214 - protein, 10 aa, Homo sapiens
VLMQDSRLYL

SEQ ID NO: 215 - protein, 9 aa, Homo sapiens
KVLEHVVRV

SEQ ID NO: 216 - protein, 9 aa, Homo sapiens
LLWGNLPEI

SEQ ID NO: 217 - protein, 9 aa, Homo sapiens
SLMEKNQSL

SEQ ID NO: 218 - protein, 9 aa, Homo sapiens
KLLAVIHEL

SEQ ID NO: 219 - protein, 10 aa, Homo sapiens
ALGDKFLLRV

SEQ ID NO: 220 - protein, 11 aa, Homo sapiens
FLMKNSDLYGA

SEQ ID NO: 221 - protein, 10 aa, Homo sapiens
KLIDHQGLYL

SEQ ID NO: 222 - protein, 12 aa, Homo sapiens
GPGIFPPPPPQP

SEQ ID NO: 223 - protein, 9 aa, Homo sapiens
ALNESLVEC

SEQ ID NO: 224 - protein, 9 aa, Homo sapiens
GLAALAVHL

SEQ ID NO: 225 - protein, 9 aa, Homo sapiens
LLLEAVWHL

SEQ ID NO: 226 - protein, 9 aa, Homo sapiens
SIIEYLPTL

SEQ ID NO: 227 - protein, 9 aa, Homo sapiens
TLHDQVHLL

SEQ ID NO: 228 - protein, 9 aa, Homo sapiens
SLLMWITQC

SEQ ID NO: 229 - protein, 11 aa, Homo sapiens
FLLDKPQDLSI

SEQ ID NO: 230 - protein, 10 aa, Homo sapiens
YLLDMPLWYL

SEQ ID NO: 231 - protein, 9 aa, Homo sapiens
GLLDCPIFL

SEQ ID NO: 232 - protein, 9 aa, Homo sapiens
VLIEYNFSI

SEQ ID NO: 233 - protein, 11 aa, Homo sapiens
TLYNPERTITV

SEQ ID NO: 234 - protein, 9 aa, Homo sapiens
AVPPPPSSV

SEQ ID NO: 235 - protein, 9 aa, Homo sapiens
KLQEELNKV

SEQ ID NO: 236 - protein, 11 aa, Homo sapiens
KLMDPGSLPPL

SEQ ID NO: 237 - protein, 9 aa, Homo sapiens
ALIVSLPYL

SEQ ID NO: 238 - protein, 9 aa, Homo sapiens
FLLDGSANV

SEQ ID NO: 239 - protein, 10 aa, Homo sapiens
ALDPSGNQLI

SEQ ID NO: 240 - protein, 9 aa, Homo sapiens
ILIKHLVKV

SEQ ID NO: 241 - protein, 9 aa, Homo sapiens
VLLDTILQL

SEQ ID NO: 242 - protein, 9 aa, Homo sapiens
HLIAEIHTA

SEQ ID NO: 243 - protein, 9 aa, Homo sapiens
SMNGGVFAV

SEQ ID NO: 244 - protein, 9 aa, Homo sapiens
MLAEKLLQA

SEQ ID NO: 245 - protein, 9 aa, Homo sapiens
YMLDIFHEV

SEQ ID NO: 246 - protein, 11 aa, Homo sapiens
ALWLPTDSATV

SEQ ID NO: 247 - protein, 9 aa, Homo sapiens
GLASRILDA

SEQ ID NO: 248 - protein, 9 aa, Homo sapiens
ALSVLRLAL

SEQ ID NO: 249 - protein, 9 aa, Homo sapiens
SYVKVLHHL

SEQ ID NO: 250 - protein, 9 aa, Homo sapiens
VYLPKIPSW

SEQ ID NO: 251 - protein, 9 aa, Homo sapiens
NYEDHFPLL

SEQ ID NO: 252 - protein, 9 aa, Homo sapiens
VYIAELEKI

SEQ ID NO: 253 - protein, 12 aa, Homo sapiens
VHFEDTGKTLLF

SEQ ID NO: 254 - protein, 9 aa, Homo sapiens
VLSPFILTL

SEQ ID NO: 255 - protein, 9 aa, Homo sapiens
HLLEGSVGV

SEQ ID NO: 256 - DNA, 3658 bp, synthetic construct
  misc_feature 1..3658: note = CD8aCD4Fusion.TCR.WPRE
  source 1..3658: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 257 - DNA, 3622 bp, synthetic construct
  misc_feature 1..3622: note = CD8bCD4Fusion.TCR.WPRE
  source 1..3622: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 258 - DNA, 3595 bp, synthetic construct
  misc_feature 1..3595: note = CD8bCD8aFusion.TCR.WPRE
  source 1..3595: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 259 - DNA, 5091 bp, synthetic construct
  misc_feature 1..5091: note = PGK.CD8.EF1a.TCR
  source 1..5091: mol_type = other DNA
tcacacgtagcgtgcggacaggctcggcttatctcctgacttcacgacaggccgcggacacttgcagactctccgcctattccggtggttgcagtacagggtgatcacaagggacaggagcagcactccacaggtgccagccaaaggggcccaaatgtagatgtcgcacgcgaagtccagaccgcgggtatgcacggctcctccagcagcaggccgacacgcttcagggcgcagcgaaaggggttgggacgcgatggtgggagcgggagttggaggtctgggtgccggagtagtggtgggcttggcaggcaggaacaccggcacgaagtgtgagaagtacatgattgaattggagagcgcagagcagaagtagtagccctcgttctcccgccggaagtcggagagagtcagcacaaaagtgtctcccagccgtttgcccgagaagcgctgggtgtccagaccctcagcggccttcggcttgttctggctcaagtacaggaggaaggtgggtgaggcggcagctcctctcggctggaacagccagctgcatccgctggtcgggttcgacaggaggacttgacacttgagctccaccgtttccccgaggttccaggtgcgatccagaggggacacccggaattgggaaggtcgtgcggcgtgcaacagcagggcaaggggcaacaggagtgcggtcacgggaagcgccatggggccagggttctcttccacgtcgccggcctgtttcagcaggctgaaattggtggcgccgctgccagatctcttggcccgggttttcaggatccaggggttcagcagcttttgcgaggtggtggtgttggaatagtagccatggaggcactgtggcacgaaagtgccactgatcccctctccctgaggctgcttcatgaacctcagtctggccctccttctccggcagcagaggtgaatggcgacgccaagggacaccagaaggaccagcactccagccacaaggagccccagggtgatcggggaacacagcggacccttctgcgtttccggccgtgggagtctgcaaacccgcttcttcagggttgacttcttagtgggttgggcggtagtggggaggaagtccaccacgctcagctgagtgcccttcccgaaggtcagttcgggtgagcccacaatcatgcaaaagtagatcccgctatcttcgggcttcacggaggtcagattcagtataaagcgggaggcatcgcgaaacacggcgatcttctcctgttccacttcttcgccgtgaatagttcccttggcggagtcccacagggccaagaactcatggtgggagtccgaggaaggcgcctgtctctgccgcaaccaatagatccgcatgttggagagggaaatcttggcctcgcaggacagcatgaccatcttgttggtctgcactttgatgtaggccggtgtctgctgcagcacgctgtttccgtgcaggacagtcagttgcgcggcgagcagaagccacagtctcgggcgcatggtggtagcgtcgacccggggcggccgctcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccagtgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctga

SEQ ID NO: 260 - DNA, 5091 bp, synthetic construct
  misc_feature 1..5091: note = PGK.TCR.EF1a.CD8
  source 1..5091: mol_type = other DNA
tcaggagctccacagcctcagggtcatcagcagattaaagccggccaccttcagcagcaggatcctgaagccgatcacgctcaggttctgaaaattcaggtttgtatcggtctcgaaagacttctccaccagcttcacgtcacaagaggactctggagaaggaaagaaggtatcctcggggatgatggagttattaaaggcattggcgcaggcgaaatcagacttattggaccaggccacggcgctgttagacttgaagtccatgcttctcatatccagcactgtcttgtcggtgatgtacacgtcgctgtccttggactggctcacgtttgtctgagaatcaaagtcggtgaacaggcacacgctcttatcgctagacttgctgtcccgcagctgatacacggcggggtctggattctggatgtttggcttcactgtcagtctggtgccggcgccaaaccgcatatcgttattgttgtacagggcgcacagataggtggcagagtcctcaggctgggagcccttgatgtacagataggagtagccctcctttgtattcagggtggcgctgatgcggcccttcttcttctcatcgccgttcagtgtcatcacgaacagggcctctggggactttgcggtctcctttctgtaccagtgcagggcgtagaagttggagctaggaaaagagcatgtgaagttggtggagtcgccctcctgcacgtgcaggctctgtgggctctgttccacattcaggatagaggacacgcagtccaggtgaaaccacaggatcagcaggggggcagccaggggattcttctccatggggccggggttctcctccacgtcgccgcaggtcagcaggctgcctctgccctcgccgctgccagatctcttggcccgtccccgactatcttttcttttgaccattgccatcagcaccagagcggagaccaggacggcgtacagtgtggccttgcccagcaggatctcatacaggatggtggcgctcagcacgccctgctggtagctctcagaggtgaagccacagtctgctcttccccatgcctcggcagacacgatctgtgtcacaggcttggcccggtcctgggtccactcatcgttctcggacaggccgtaaaactgcacctgacatctgaagtgattccgggggttctgccaaaaggtggcggacactctcagccgagaggacaggcaataccggctatcattcagggctggctgctccttcaggggctgtgggtctgtggacacgccgctgtgcacctccttgccgttcacccaccaggacagctccacgtgatcaggatagaagccggttgccaggcacaccagggtggccttctgggtgtggctgatctcggcctcagagggctcaaacacggccacctcaggggggaacacgttcttcaggtcctccagcactgtcagcctggttcctggtccgaagtactgtgtatcggtgctgcctgggctagaggcgcaaaaatacacggcggagtctcttggctcgctaggctggatcttcagtgtggagaagctggcattgggcatcttggcagaaaaccgatcctcaggcatgccggaatcgtcgatgggcacattgttattgaagtagatcagcagctccaggccgcgcatcatggtctccctataccaaaacaggctgttgtggccagagattggcttacagcgcagtgtcacctcctggcccatctcggtcacctcgtgcctaggggactggatcacgccggcgtctgtgtgcttggccaccaggatgcacaggctcacgcagcagaaggtccaagagtccatggtggtagcgtcgacccggggcggccgctcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccagtgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacagtgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgtga

SEQ ID NO: 261 - DNA, 2749 bp, synthetic construct
  misc_feature 1..2749: note = NFAT.IL12
  source 1..2749: mol_type = other DNA
gtcgaccgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgcattttgacacccccataatatttttccagaattaacagtataaattgcatctcttgttcaagagttccctatcactctctttaatcactactcacagtaacctcaactcctgcccaagcttggcattccggtactgttggtaaagccaccatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaacaagccagcattagcgtgcgggcccaggaccgctactatagctcatcttggagcgagtgggcatctgtgccctgcagtggcggcggcggcagcggcggcggcggcagcggcggcggcggcagcatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctagtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 262 - DNA, 5048 bp, synthetic construct
  misc_feature 1..5048: note = CD69pro.IL12
  source 1..5048: mol_type = other DNA
tttatgatagcatagtagcccagaccccggcctaccttaaaattctatatggtacatttgctattttcacaccattatctttgagagtagtctccaaaaatgttgtatatggataagtctggaattaatttaggtaaagtactcagacatggttaagcgttatatctcatctttaatatagctcttcttttattctcttatgtattttactttcagaaacagtcaggtttttcagagtccaagcttgtgtgtacacaagcactatcttttaccttcactgtctcaagtcaaaaagaagaaaatgccagctcacactttccaagctcattttttgtgtttcagatgttccattgaagaggaaagttagaaagttcaaagacttgaagaggacagaaggttggaaagtgtttaaaactggaaatcccccgtttactcatggttaccttcattgaccctttacggtaaacaattcaaaaacacaaaggcacctgcaggttaaaaataaattttaccaatttgtgtaatttgcattaatttgagagaggatgatgtattctaaattgaagttttgattcacaagaagattaaaagccattcagaaacctaattcacccactgaaaggaaaaaaaaaaaaagagagatgagcagtttgtctccggaaattgtcttaggtcggaagtctgtggtccctgttcacatgtacccaaaagcatcctgctgctgcagctgtctgataagcacagagtaccccaccttctctgcacactttgcatctagctcatattacctcatctttacttcctttctgacgtctcaccctggattctacatataaggtcacacaggaaggaaagctgcattgagttttggtgtcctgaaagacttttgccaaccttgtccccgcactaatttctctaagcctcggctatactattttctcagctacacgatgaaatgtgaatgataatttctgccctaaaaatatcacttaattttttaacatatcatttatgaaagaagacacataaaatgtctccctgaagagtgagtcggttaaagggagagcgagacatgcagggagatggaaaaagaatcttttagagaaaaagtaaaagccctgtagtggtaggatgcttggcatttttaaggaggaggtttctgtcagagaggatactgaataataggaaaaatgataagagaggtaaccaggggccatattatgtataaagcattctcaactattaggtctttggattttacggtacatttgataggaagcctttcaaagtgcagtggggggaggagttaaacacttcaccaaaatcatatgaattaattataaaacaatggttctagctgctatgtggaaaagggactaagaaattgactgaaaaataggatggaagcaaagggagcttttagggccaacatgtacctctttgtgtcttaggtagacaaatgcatctaatggtccaactaacttctctagatgataacttccaacccacctatgcataaaatttaacgtctttattctaaataagtgatattaataataaaatttggggcaccaagattattaatcagagtggtattttgatttccctccttaaatcaccatacatagctttctgcattcatcttgcgttgactgtcattacttgtctgagtgagactgataccacagcgatgttttaaataataatcatacctcaaaagactgaagtctcagaggtatctgaagagaataacctagagcacagggggagaattgaaggagctgttactgaggtgacataaaagcagtctaaatgacagtaaaatgtgacaagaaaattagcaggaaacaaatgaaacagataatttaagataaacaattttagagcatagcaaggaagttccagaccacaagctttctgtttcctgcattcttacttcttactacgtgatacatctagtcaccagggaagaagcgaatgacacacttccaaaaaccaattcgtagctttctaaataaaaccctttcaagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcacagactcaacaagagctccagcaaagactttcactgtagcttgacttgacctgagattaactagggaatcttgacagcggccgccccgggtcgacgctaccaccatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaacaagccagcattagcgtgcgggcccaggaccgctactatagctcatcttggagcgagtgggcatctgtgccctgcagtggcggcggcggcagcggcggcggcggcagcggcggcggcggcagcatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctagtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 263 - DNA, 1540 bp, synthetic construct
  misc_feature 1..1540: note = NFAT.IL18.var1
  source 1..1540: mol_type = other DNA
gtcgaccgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgcattttgacacccccataatatttttccagaattaacagtataaattgcatctcttgttcaagagttccctatcactctctttaatcactactcacagtaacctcaactcctgcccaagcttggcattccggtactgttggtaaagccaccatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 264 - DNA, 3839 bp, synthetic construct
  misc_feature 1..3839: note = CD69pro.IL18.var1
  source 1..3839: mol_type = other DNA
tttatgatagcatagtagcccagaccccggcctaccttaaaattctatatggtacatttgctattttcacaccattatctttgagagtagtctccaaaaatgttgtatatggataagtctggaattaatttaggtaaagtactcagacatggttaagcgttatatctcatctttaatatagctcttcttttattctcttatgtattttactttcagaaacagtcaggtttttcagagtccaagcttgtgtgtacacaagcactatcttttaccttcactgtctcaagtcaaaaagaagaaaatgccagctcacactttccaagctcattttttgtgtttcagatgttccattgaagaggaaagttagaaagttcaaagacttgaagaggacagaaggttggaaagtgtttaaaactggaaatcccccgtttactcatggttaccttcattgaccctttacggtaaacaattcaaaaacacaaaggcacctgcaggttaaaaataaattttaccaatttgtgtaatttgcattaatttgagagaggatgatgtattctaaattgaagttttgattcacaagaagattaaaagccattcagaaacctaattcacccactgaaaggaaaaaaaaaaaaagagagatgagcagtttgtctccggaaattgtcttaggtcggaagtctgtggtccctgttcacatgtacccaaaagcatcctgctgctgcagctgtctgataagcacagagtaccccaccttctctgcacactttgcatctagctcatattacctcatctttacttcctttctgacgtctcaccctggattctacatataaggtcacacaggaaggaaagctgcattgagttttggtgtcctgaaagacttttgccaaccttgtccccgcactaatttctctaagcctcggctatactattttctcagctacacgatgaaatgtgaatgataatttctgccctaaaaatatcacttaattttttaacatatcatttatgaaagaagacacataaaatgtctccctgaagagtgagtcggttaaagggagagcgagacatgcagggagatggaaaaagaatcttttagagaaaaagtaaaagccctgtagtggtaggatgcttggcatttttaaggaggaggtttctgtcagagaggatactgaataataggaaaaatgataagagaggtaaccaggggccatattatgtataaagcattctcaactattaggtctttggattttacggtacatttgataggaagcctttcaaagtgcagtggggggaggagttaaacacttcaccaaaatcatatgaattaattataaaacaatggttctagctgctatgtggaaaagggactaagaaattgactgaaaaataggatggaagcaaagggagcttttagggccaacatgtacctctttgtgtcttaggtagacaaatgcatctaatggtccaactaacttctctagatgataacttccaacccacctatgcataaaatttaacgtctttattctaaataagtgatattaataataaaatttggggcaccaagattattaatcagagtggtattttgatttccctccttaaatcaccatacatagctttctgcattcatcttgcgttgactgtcattacttgtctgagtgagactgataccacagcgatgttttaaataataatcatacctcaaaagactgaagtctcagaggtatctgaagagaataacctagagcacagggggagaattgaaggagctgttactgaggtgacataaaagcagtctaaatgacagtaaaatgtgacaagaaaattagcaggaaacaaatgaaacagataatttaagataaacaattttagagcatagcaaggaagttccagaccacaagctttctgtttcctgcattcttacttcttactacgtgatacatctagtcaccagggaagaagcgaatgacacacttccaaaaaccaattcgtagctttctaaataaaaccctttcaagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcacagactcaacaagagctccagcaaagactttcactgtagcttgacttgacctgagattaactagggaatcttgacagcggccgccccgggtcgacgctaccaccatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagtgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 265 - DNA, 607 bp, synthetic construct
  misc_feature 1..607: note = WPREmut1
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 266 - DNA, 581 bp, synthetic construct
  misc_feature 1..581: note = WPREmut2
  source 1..581: mol_type = other DNA
gagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc